(19) United States
(12) Patent Application Publication
(10) Pub. No.: US 2007/0191364 A1
(43) Pub. Date: Aug. 16, 2007

(54) AMINOPIPERIDINE DERIVATIVES, PREPARATION THEREOF AND THERAPEUTIC USE THEREOF

(75) Inventors: Alain BRAUN, Boulogne Billancourt (FR); Bruno CORNET, Perthes-En-Gatinais (FR); Gilles COURTEMANCHE, Saubens (FR); Olivier CRESPIN, Cergy (FR); Eykmar FETT, 75016 (FR); Cecile PASCAL, Rueil-Malmaison (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/626,972

(22) Filed: Jan. 25, 2007

Related U.S. Application Data
(63) Continuation of application No. PCT/FR05/01855, filed on Jul. 20, 2005.

(30) Foreign Application Priority Data
Jul. 29, 2004 (FR) 0408370

(51) Int. Cl. A61K 31/5377; A61K 31/496; A61K 31/4747; A61K 31/454; A61K 31/4545; C07D 413/02; C07D 405/02; C07D 401/14

(52) U.S. Cl. 514/235.2; 514/304; 514/317; 546/226; 514/253.12; 544/360; 514/326; 546/208; 514/316; 546/187; 546/189; 544/129; 514/278; 546/15

(57) ABSTRACT

The present invention relates to compounds of formula (I) as defined herein that are melanocortin receptor agonists, to the preparation thereof and to the therapeutic use thereof in the treatment and in the prevention of obesity, diabetes and sexual dysfunctions that can affect both sexes, in the treatment of cardiovascular diseases, and also in anti-inflammatory uses or in the treatment of alcohol dependency.

### BACKGROUND OF THE INVENTION

The present invention relates to compounds that are melanocortin receptor agonists, to the preparation thereof and to the therapeutic use thereof.

Melanocortin receptors (MC-Rs) belong to the superfamily of G protein-coupled seven-transmembrane domaine receptors. Their transduction pathway involves the production of cAMP (Cone, R. D., Recent Prog. Horm. Res., 1996, 51, 287). Five MC-R subtypes have currently been described, MC1-R, MC2-R, MC3-R, MC4-R and MC5-R, and are expressed in various tissues, such as the brain (MC3, 4, 5-R), the exocrine glands (MC5-R), the adrenals (MC2-R) and the skin (MC1-R), as regards the main ones. The natural ligands of MC-Rs are, as regards the agonists, ACTH, and α-, β- and γ-MSH, and as regards the antagonists, agouti protein and agouti-related protein. None of the natural ligands is very selective for one of the subtypes, with the exception of γ-MSH, which have a certain selectivity for MC3-R.

The melanocortin system is involved in many physiological processes, including pigmentation, inflammation, eating behaviour and sexual behaviour (in particular erectile function), energetic balance (regulation of body weight and lipid storage), exocrine functions, neuronal protection and regeneration, immunomodulation, analgesia, etc.

In particular, it has been demonstrated that MC4-R is involved in sexual behaviour (Van der Ploeg, L. H., Proc. Natl. Acad. Sci. USA, 2002, 99, 11381; Martin, W. J., Eur. J. Pharmacol., 2002, 454, 71). It has also been demonstrated, by means of mouse models specifically devoid of certain MC-Rs (knockout mice), that the central MC-Rs (MC3 and 4-R) are involved in eating behaviour, obesity, the metabolism and energetic balance (Huszar, D., Cell, 1997, 88(1), 131; Chen, A. S., Nat. Genet., 2000, 26(1), 97; Butler, A. A., Trends Genet., 2001, 17, pp. 50-54). Thus, MC4-R knockout mice are hyperphagic and obese. In parallel, MC3 and/or 4R antagonists promote food intake, whereas the stimulation of MC4-Rs by an endogenous agonist, such as α-MSH, produces a satiety signal.

These observations imply that the stimulation of central MC3-R and/or MC4-R, reducing food intake and body weight, is a promising approach for treating obesity, which is an aggravating risk for many other pathologies (hypertension, diabetes, etc.). Thus, research studies have made it possible to identify, initially, peptides, pseudopeptides or cyclic peptides capable of interacting with MC-Rs and of thus modulating food intake.

In order to maintain an effective weight loss in the long term and thus to limit comorbidities, a long-term daily treatment must be envisaged. This implies that a medicament, for this therapeutic indication, must be able to be administered simply by the patient. Oral administration must therefore be favoured. Now, peptide compounds are not generally the most suitable for satisfying this need. For this reason, it is important to develop small non-peptide molecules.

In this perspective, international PCT applications published under the numbers WO 02/059095, WO 02/059108, WO 03/009850 and WO 03/061660 describe piperazine-type derivatives. Other applications describe piperidine-type derivatives, such as WO 03/092690 and WO 03/093234. Applications WO 99/64002 and WO 01/70337 describe spiropiperidine-type derivatives. Application WO 01/91752 describes derivatives containing a piperidine unit fused with a pyrazolyl ring. Application WO 02/059107 describes piperidine-type and piperazine-type derivatives substituted with a bicyclic structure. Applications WO 02/059117, WO 02/068388 and WO 03/009847 describe piperidine-type and/or piperazine-type derivatives substituted with a phenyl ring. As regards application WO 03/094918, it describes piperazine-type derivatives substituted with a phenyl or pyridinyl ring. Mention may also be made of applications WO 00/74679, WO 01/70708, WO 02/15909, WO 02/079146, WO 03/007949 and WO 04/024720, which describe substituted piperidine-type derivatives, or else application WO 04/037797; the compounds described in those patent applications always contain an amide function, that mimics the peptide structures previously known.

Mention may also be made of WO 2005/0472533, which describes compounds that are melanocortin receptor agonists, of general formula:

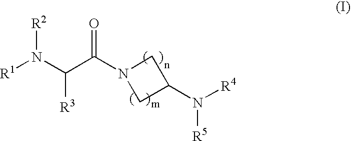

Faced with the constant need to improve existing therapies for the pathologies mentioned above, the inventors gave themselves the aim of providing novel compounds that are melanocortin receptor agonists.

### SUMMARY OF THE INVENTION

A subject of the present invention is compounds corresponding to formula (I)

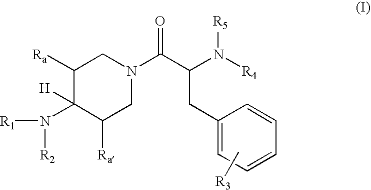

in which:

Rₐ and R_{a′}, which may be identical to or different from one another, represent a hydrogen atom, or an alkyl or cycloalkyl group,

R₁ represents a hydrogen atom, or an alkyl, cycloalkyl, heterocycloalkyl or aryl group,

R₂ represents a group of formula —(CH₂)ₓ—(co)_{y}—Y or —(CO)_{y}—(CH₂)ₓ—Y, in which:
x=0, 1, 2, 3 or 4,
y=0 or 1,
Y represents a hydrogen atom, or a hydroxyl, alkyl, cycloalkyl, alkoxy, aryl, heteroaryl or —NR₁₁R₁₂ group, Y being different from a hydrogen atom when x=y=0,
R₁₁ and R₁₂, which may be identical to or different from one another, represent a hydrogen atom, or an alkyl, cycloalkyl, alkoxy or —NR₁₃R₁₄ group, or else R₁₁ and R₁₂ form, together with the nitrogen atom to which they are attached, a mono- or bicyclic structure containing from 4 to 10 ring members and optionally comprising 1 to 3 additional hetero atoms and/or 1 to 3 ethylenic or acetylenic unsaturations, this ring being optionally substituted in any positions with 1 to 3 groups chosen from halogen atoms, and hydroxyl, alkyl, cycloalkyl and alkoxy groups. By way of examples of such cyclic structures, mention may be made of pyrrolidinyl, morpholinyl, pyrrolinyl, isoindolinyl groups, etc.,

R₁₃ and R₁₄, which may be identical to or different from one another, represent a hydrogen atom, or an alkyl, cycloalkyl or alkoxy group, or else R₁₃ and R₁₄ form, together with the nitrogen atom to which they are attached, a mono- or bicyclic structure as defined above,

R₃ represents 1 to 3 groups, which may be identical to or different from one another, located in any positions of the ring to which they are attached and chosen from halogen atoms, and alkyl, cycloalkyl, —OR, —NRR′, —CO—NRR′, —NR—CO—R′, —NR—CO—NRR′, —NR—COOR′, —NO₂, —CN and —COOR groups, where R and R′ are as defined below,

R₅ represents a hydrogen atom or an alkyl group,

R₄ is chosen from the groups of formulae (a), (b) and (c), optionally substituted with an oxo group or mono- or polysubstituted with an aryl or heteroaryl group below (each of these cyclic structures (a), (b) and (c) being directly attached to the nitrogen atom of formula (I) that carries it):

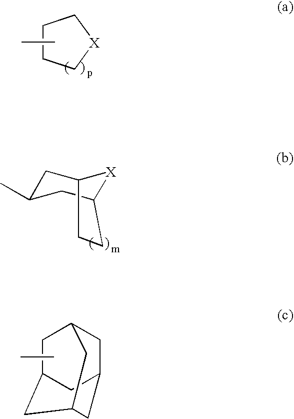

in which:

p=0, 1, 2 or 3,

m=0, 1 or 2,

and either

a) X represents a ring member —N(R₁₀)—, where

R₁₀ is chosen from:

a group —(CH₂)ₓ—OR₈, —(CH₂)ₙ—COOR₈, —(CH₂)ₓ—NR₈R₉, —(CH₂)—CO—NR₈R₉ or —(CH₂)ₓ—NR₈—COR₉, —(CH₂)ₓ—COR₈ in which x=1, 2, 3 or 4,
a cycloalkyl or heterocycloalkyl group fused with an aryl or heteroaryl group,

a cycloalkyl, heterocycloalkyl, aryl, hetero-aryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉, —C (═NH)—NR₈R₉, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl or —SO₂—NR₈R₉ group,

the alkyl, cycloalkyl, heterocycloalkyl, aryl or heteroaryl groups being optionally substituted with 1 or more groups chosen from the groups R, R′, —OR, —NRR′, —CO—NRR′, —NR—CO—R′, —NR—CO—NRR′, —NO₂, —CN and —COOR, OCOR, COR, OCONRR′, NRCOOR′,

the cycloalkyl or heterocycloalkyl groups being optionally fused with an aryl or heteroaryl group,

or else R₁₀ forms, with the nitrogen atom to which it is attached and a carbon atom located in any position of the cyclic structure of formula (a), but not adjacent to said nitrogen atom, a bridge comprising from 3 to 5 members,

R₈ and R₉ are chosen, independently of one another, from a hydrogen atom, and alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4,

or else R₈ and R₉ together form a cycloalkyl or a heterocycloalkyl;

R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl; or,

b) X represents a ring member —C(R₆)(R₇)—, where

R₆ is chosen from:
a hydrogen atom, a halogen atom,
a group —(CH₂)ₓ—OR₈, —(CH₂)ₓ—COOR₈, —(CH₂)ₓ—NR₈R₉, —(CH₂)ₙ—CO—NR₈R₉ or —(CH₂)ₓ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉ or —C(═NH)—NR₈R₉ group,
a fused or nonfused cycloalkyl or heterocycloalkyl group located in the spiro position on the ring of formula (a) to which it is attached,
a cycloalkyl or heterocycloalkyl group fused with an aryl or heteroaryl group,
the alkyl, cycloalkyl, heterocycloalkyl, aryl or heteroaryl groups being optionally substituted with 1 or more groups chosen from the groups R, R′, —OR, —NRR′, —CO—NRR′, —NR—CO—R′, —NR—CO—NRR′, —NO₂, —CN, —COOR, OCOR, COR, OCONRR′, NRCOOR′;

the cycloalkyl or heterocycloalkyl groups being optionally fused with an aryl or heteroaryl group,

R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkylaryl, —O-alkylheteroaryl, —NRR′, —CO—NRR′, —NR—CO—R′, —NR—CO—NRR′, —NR—COOR′, —NO₂, —CN and —COOR groups,
R₈ and R₉ are chosen, independently of one another, from a hydrogen atom, and alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4, the alkyl and aryl groups being optionally substituted with one or more groups chosen from the groups R, R′, —OR, —NRR′, —CO—NRR′, —NR—CO—R′, —NR—CO—NRR′, —NO₂, —CN and —COOR, OCOR, COR, OCONRR′, NRCOOR′,

or else R₈ and R₉ together form a cycloalkyl or a heterocycloalkyl;

R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl,

in the form of a base or of an addition salt with an acid, and also in the form of a hydrate or of a solvate.

### DETAILED DESCRIPTION

Preferably, when X represents a ring member —C(R₆)(R₇) in the compounds of formula (I), R₆ and R₇ do not represent at the same time a hydrogen atom.

Among the compounds of formula (I) that are subjects of the invention, preference is given to those in which R₄ is chosen from the groups of formulae (a), (b) and (c), optionally mono- or polysubstituted with an aryl or heteroaryl group where X represents a ring member —C(R₆) (R₇)—, in which R₆ is chosen from:
a hydrogen atom,
a group —(CH₂)ₙ—OR₈, —(CH₂)—COOR₈, —(CH₂)ₓ—NR₈R₉, —(CH₂)—CO—NR₈R₉ or —(CH₂)ₓ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkylheteroaryl group,
a cycloalkyl or heterocycloalkyl group located in the spiro position on the ring of formula (a) to which it is attached,
a cycloalkyl or heterocycloalkyl group fused with an aryl or heteroaryl group,

R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkyl-aryl, —O-alkylheteroaryl, —NRR′, —CO—NRR′, —NR—CO—R′, —NR—CO—NRR′, —NR—COOR′, —NO₂, —CN and —COOR groups,

R₈ and R₉ are chosen, independently of one another, from a hydrogen atom, and alkyl, cycloalkyl, heterocycloalkyl, aryl, heteroaryl, alkylaryl, alkyl-heteroaryl, —CO-alkyl, —CO-cycloalkyl, —CO-hetero-cycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4;

R and R′ represent, independently of one another, a hydrogen atom, or an alkyl, cycloalkyl, heterocycloalkyl, aryl, heteroaryl, alkylaryl or alkyl-heteroaryl group.

Among the compounds of formula (I) that are subjects of the invention, further preference is given to those in which R₄ is chosen from the groups of formulae (a), (b) and (c) where X represents a ring member —C(R₆) (R₇)—, in which R₆ is chosen from a halogen atom, or a fused or nonfused cycloalkyl or heterocyclo-alkyl group located in the spiro position on the ring of formula (a) to which it is attached.

Among the compounds of formula (I) that are subjects of the invention, further preference is given to those in which R₄ is chosen from the groups of formulae (a), (b) and (c) where X represents a ring member —C(R₆)(R₇)—, in which R₆ is chosen from —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉ and —C(═NH)—NR₈R₉.

Among the compounds of formula (I) that are subjects of the invention, further preference is given to those in which R₄ is chosen from the groups of formulae (a), (b) and (c) where X represents a ring member —C(R₆)(R₇)—, in which the alkyl, cycloalkyl, heterocycloalkyl, aryl or heteroaryl groups are optionally substituted with 1 or more groups chosen from R, R′, OCOR, COR, OCONRR′ and NRCOOR′.

Among the compounds of formula (I) that are subjects of the invention further preference is given to those in which R₄ is chosen from the groups of formulae (a), (b) and (c) where X represents a ring member —C(R₆) (R₇)—, in which the cycloalkyl or heterocycloalkyl groups are optionally fused with an aryl or heteroaryl group.

Among the compounds of formula (I) that are subjects of the invention further preference is given to those in which R₄ is chosen from the groups of formulae (a), (b) and (c) where X represents a ring member —C(R₆)(R₇)—, in which R₈ and R₉, chosen independently of one another, represent alkyl and aryl groups which are optionally substituted with one or more groups chosen from the groups R, R′, OCOR, COR, OCONRR′ or NRCOOR′.

Among the compounds of formula (I) that are subjects of the invention further preference is given to those in which R₄ is chosen from the groups of formulae (a), (b) and (c) where X represents a ring member —C(R₆) (R₇)—, in which R and R′ can together form a cycloalkyl or a heterocycloalkyl.

Preferably, in the compounds of formula (I), R₇ is hydrogen.

Further preference is given to those in which R₄ represents the group of formula a) where p=2 as defined below:

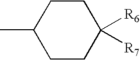

Further preference is given to those in which R₄ is chosen from the groups of formulae (a), (b) and (c), optionally mono- or polysubstituted with an aryl or heteroaryl group, where X represents a ring member —N(R₁₀)— in which

R₁₀ is chosen from:

a group —CO—NR₈R₉, —COOR₈,

a group —(CH₂)ₓ—OR₈, —(CH₂)ₓ—COOR₈, —(CH₂)ₓ—NR₈R₉, —(CH₂)ₓ—CO—NR₈R₉ or —(CH₂)—NR₈—COR₉, in which x=1, 2, 3 or 4,
a cycloalkyl or heterocycloalkyl group fused with an aryl or heteroaryl group,

a cycloalkyl, heterocycloalkyl, aryl, heteroaryl, alkylaryl, alkylheteroaryl, —CO-cycloalkyl, —CO-heterocycloalkyl, —CO-heteroaryl, —CO-alkylaryl, —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉, —C (═NH)—NR₈R₉, —SO₂-cycloalkyl, —SO₂-heterocycloalkyl, —SO₂-heteroaryl, —SO₂-alkylaryl, —SO₂-alkylheteroaryl or —SO₂—NR₈R₉ group;

or else R₁₀ forms, with the nitrogen atom to which it is attached and a carbon atom located in any position of the cyclic structure of formula (a), but not adjacent to said nitrogen atom, a bridge comprising from 3 to 5 members;

R₈ and R₉ are chosen, independently of one another, from a hydrogen atom, and alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4;

R and R′ represent, independently of one another, a hydrogen atom, or an alkyl, cycloalkyl, heterocycloalkyl, aryl, heteroaryl, alkylaryl or alkylheteroaryl group.

Further preference is given to those in which R₄ is chosen from the groups of formulae (a), (b) and (c) optionally substituted with an oxo group where X represents a ring member —N(R₁₀).

Among the compounds of formula (I) that are subjects of the invention further preference is also given to those in which R₄ is chosen from the groups of formulae (a), (b) and (c) where X represents a ring member —N(R₁₀)—, in which

R₈ and R₉, together form a cycloalkyl or a heterocycloalkyl.

Further preference is given to those in which R₄ is chosen from the groups of formulae (a), (b) and (c) where X represents a ring member —N(R₁₀)—, in which

R₁₀ is —(CH₂)ₙ—COR₈, in which x=1, 2, 3 or 4.

Further preference is given to those in which R₄ is chosen from the groups of formulae (a), (b) and (c) where X represents a ring member —N(R₁₀)—, in which the alkyl, cycloalkyl, heterocycloalkyl, aryl or heteroaryl groups are optionally substituted with one or more groups chosen from R, R′ OCOR, COR, OCONRR′ or NRCOOR′.

Further preference is given to those in which R₄ is chosen from the groups of formulae (a), (b) and (c) where X represents a ring member —N(R₁₀)—, in which the cycloalkyl or heterocycloalkyl groups are optionally fused with an aryl or heteroaryl group.

Further preference is given to those in which R₄ represents the group of formula a) where p=2 as defined below:

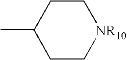

The compounds of formula (I) contain at least one asymmetric carbon atom. They can therefore exist in the form of enantiomers or of diastereoisomers. These enantiomers and diastereoisomers, and also mixtures thereof, including racemic mixtures, are part of the invention.

Among the compounds of formula (I) that are subjects of the invention, preference is given to those in which the carbon atom identified by the asterisk * in the formula below is in an (R) configuration:

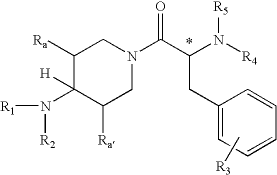

The compounds of formula (I) according to the invention can also exist in the form of mixtures of conformers, which are part of the invention. They can also exist in the form of cis or trans isomers, or in the form of endo or exo isomers. These isomers, and also the mixture thereof, are part of the invention.

The compounds of formula (I) can exist in the form of bases or of addition salts with acids. Such addition salts are part of the invention.

These salts are advantageously prepared with pharmaceutically acceptable acids, but the salts of other acids that are useful, for example, for purifying or isolating the compounds of formula (I) are also part of the invention.

The compounds of formula (I) can also exist in the form of hydrates or of solvates, i.e. in the form of associations or of combinations with one or more molecules of water or with a solvent. Such hydrates and solvates are also part of the invention.

In the context of the present invention, and unless otherwise mentioned in the text, the term:
“a halogen atom” is intended to mean: a fluorine, a chlorine, a bromine or an iodine;
“an alkyl group” is intended to mean: a saturated or unsaturated (i.e. comprising between 1 and 3 unsaturations of ethylenic or acetylenic type), linear, cyclic or branched aliphatic group comprising from 1 to 6 carbon atoms. By way of examples, mention may be made of methyl, ethyl, propyl, isopropyl, butyl, isobutyl, tert-butyl, pentyl, neopentyl groups, etc., and the cycloalkyl groups defined below, and also alkyl groups only partially cyclized, such as the methylcyclopropyl group. Such an alkyl group may be substituted with 1 or more groups (for example with 1 to 6 groups) chosen from halogen atoms (resulting, for example, in a —CF₃ group) and the groups R, R′, —OR, —NRR′, —CO—NRR′, —NR—CO—R′, —NR—CO—NRR′, —NO₂, —CN and —COOR, OCOR, COR, OCONRR′, NRCOOR′; where R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl;
“a cycloalkyl group” is intended to mean: a cyclic alkyl group comprising between 3 and 8 carbon atoms, all the carbon atoms being involved in the cyclic structure. By way of examples, mention may be made of cyclopropyl, cyclobutyl, cyclopentyl, cyclohexyl groups, etc. Such a cycloalkyl group may be substituted with R, R′ and as described above for the alkyl group;
“a heterocycloalkyl group” is intended to mean: a cycloalkyl group as defined above, also comprising between 1 and 4 hetero atoms, such as nitrogen, oxygen and/or sulphur. Such a heterocycloalkyl group may be substituted as described above for the cycloalkyl group and may comprise one or more, for example 1 or 2, ethylenic or acetylenic unsaturations. By way of examples of heterocycloalkyl groups, mention way be made of piperidinyl and tetrahydropyran groups;
“an alkoxy group” is intended to mean: an —O-alkyl radical, where the alkyl group is as defined above;
“an aryl group” is intended to mean: a cyclic aromatic group comprising between 5 and 10 ring members, for example a phenyl group. Such an aryl group may be substituted with 1 or more groups (for example with 1 to 6 groups) chosen from halogen atoms (resulting, for example, in a —CF₃ group), and the groups R, R′, —OR, —NRR′, —CO—NRR′, —NR—CO—R′, —NR—CO—NRR′, —NO₂, —CN and —COOR, OCOR, COR, OCONRR′, NRCOOR′; where R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl;
“an alkylaryl group” is intended to mean: an alkyl group as defined above, itself substituted with an aryl group as defined above. Such an alkylaryl group is, for example, a benzyl group;
“a heteroaryl group” is intended to mean: a cyclic aromatic group comprising between 5 and 10 ring members and comprising between 1 and 6 hetero atoms, such as nitrogen, oxygen and/or sulphur. By way of example, mention may be made of the pyridinyl group. Such a heteroaryl group may be substituted as described above for the aryl group;
“an alkylheteroaryl group” is intended to mean: an alkyl group as defined above, itself substituted with a heteroaryl group as defined above.

Among the compounds of formula (I) that are subjects of the invention, mention may be made of those in which Rₐ, R_{a′}, R₂, R₃, R₄ and R₅ are as defined above and R₁ represents an alkyl, cycloalkyl or heterocycloalkyl group. Advantageously, R₁ represents a cycloalkyl group, such as a cyclohexyl or cycloheptyl group.

Among the compounds of formula (I) that are subjects of the invention, mention may also be made of those in which Rₐ, R_{a′}, R₁, R₃, R₄ and R₅ are as defined above and R₂ is chosen from the following groups: —CO—R₁₅, —CO—NR₁₆R₁₇, —CO—NR₁₅—NR₁₆R₁₇, —CO-aryl, —CO-heteroaryl, —CO— (CH₂)ₓ—NR₁₆R₁₇, —(CH₂)ₓ—NR₁₆R₁₇, —(CH₂)ₓ—OH, —(CH₂)ₓ-aryl, —(CH₂)ₓ-heteroaryl, —(CH₂)ₓ—CO—R₁₅ and —(CH₂)ₓ—CO—NR₁₆R₁₇, in which:
x=0, 1, 2, 3 or 4 and x′=1, 2, 3 or 4,
R₁₅ represents a hydrogen atom, or an alkyl, cycloalkyl or alkoxy group, and
R₁₆ and R₁₇, which may be identical to or different from one another, represent a hydrogen atom, or an alkyl, cycloalkyl or alkoxy group, or else R₁₆ and R₁₇ form, together with the nitrogen atom to which they are attached, a mono- or bicyclic structure containing from 4 to 10 ring members and optionally comprising 1 to 3 additional hetero atoms and/or 1 to 3 ethylenic or acetylenic unsaturations, this ring being optionally substituted in any positions with 1 to 3 groups chosen from halogen atoms, and hydroxyl, alkyl, cycloalkyl and alkoxy groups.

Among these compounds, mention may in particular be made of those in which R₂ is chosen from the following groups: —CO—R₁₅, —CO—NR₁₆R₁₇, —CO—NR₁₅—NR₁₆R₁₇, —CO— (CH₂)ₓ—NR₁₆R₁₇, —(CH₂)ₓ—NR₁₆R₁₇, —(CH₂)ₓ—OH, —(CH₂)ₓ-aryl, —(CH₂)ₓ-heteroaryl, —(CH₂)ₓ—CO—R₁₅ and —(CH₂)ₓ, —CO—NR₁₆R₁₇, in which x, x′, R₁₅, R₁₆ and R₁₇ are as defined above.

Among the compounds of formula (I) that are subjects of the invention, mention may more particularly be made of those in which R₂ represents a group —CO—NR₁₆R₁₇, where R₁₆ and R₁₇ represent alkyl or alkoxy groups.

Among the compounds of formula (I) that are subjects of the invention, mention may also be made of those in which Rₐ, R_{a′}, R₁, R₂, R₄ and R₅ are as defined above and R₃ represents 1 to 3 groups, which may be identical to or different from one another, chosen from halogen atoms. Advantageously, R₃ represents a single group, preferably a chlorine atom.

Among the compounds of formula (I) that are subjects of the invention, mention may also be made of those in which Rₐ, R_{a′}, R₁, R₂, R₃ and R₄ are as defined above and R₅ represents a hydrogen atom or an alkyl group comprising from 1 to 4 carbon atoms. R₅ preferably represents a hydrogen atom.

Among the compounds of formula (I) that are subjects of the invention, mention may be also made of those in which R₁, R₂, R₃, R₄ and R₅ are as defined above, and Rₐ and R_{a′} represent hydrogen atoms, or alkyl groups comprising from 1 to 4 carbon atoms. Advantageously, Rₐ and R_{a′} represent, independently of one another, hydrogen atoms or methyl groups.

Among the groups R₆ defined above, mention may in particular be made of those in which R₆ represents a hydrogen atom, or an —OR₈, —NR₈R₉ or —NR₈—CO—R₉ group, in which R₈ and R₉ represent a hydrogen atom or an alkyl group.

Among the groups R₇ defined above, mention may in particular be made of those in which R₇ represents a hydrogen or halogen atom, or an alkyl group, hydroxyl group (corresponding to a group —OR, where R represents a hydrogen atom) or alkoxy group (corresponding to a group —OR, where R represents an alkyl group). R₇ advantageously represents a hydrogen atom.

Among the groups R₈ and R₉ defined above, mention may in particular be made of those in which R₈ and R₉ represent a hydrogen atom or an alkyl group.

Among the groups R₁₀ defined above, mention may in particular be made of those in which R₁₀ represents a hydrogen atom, or an alkyl or —CO-aryl group (such as —CO-phenyl), or else R₁₀ forms, with the nitrogen atom to which it is attached and a carbon atom located in any position of the cyclic structure that carries it (such as the structure of formula (a) or (a-3)), but not adjacent to said nitrogen atom, a bridge comprising from 3 to 5 members.

Among the groups R and R′ defined above, mention may in particular be made of those in which R and R′ represent a hydrogen atom or an alkyl group.

Each of the definitions given above for the groups Rₐ, R_{a′}, R₁, R₂, R₃, R₄, R₅, R₆, R₇, R₈, R₉, R₁₀, R and R′ can be combined with one another so as to obtain various subgroups of compounds of formula (I) according to the present invention.

According to another subject, the invention relates to the compounds having the following names:

In the lists that follow, the numbers in front of the names of the products correspond to the example Nos. of the compounds in the table:
2: N-{1-[1N-(4-aminocyclohexyl)-4-chloro-D-phenylalanyl]piperidin-4-yl}-N-cyclohexyl-N′,N′-diethylurea
8: N-{1-[N-(cis-4-aminocyclohexyl)-4-chloro-D-phenylalanyl]piperidin-4-yl}-N-cyclohexyl-1,3-dihydro-2H-isoindole-2-carboxamide
9: N-{1-[N-(cis-4-aminocyclohexyl)-4-chloro-D-phenylalanyl]piperidin-4-yl}-N-cyclohexyl-2,5-dimethylpyrrolidine-1-carboxamide
12: N-{1-[N-(cis-4-aminocyclohexyl)-4-chloro-D-phenylalanyl]piperidin-4-yl}-N-cyclohexyl-N′,N′-dimethylurea
14: N-{1-[N-(cis-4-aminocyclohexyl)-4-chloro-D-phenylalanyl]piperidin-4-yl}-N-cyclohexyl-N′-methoxy-N′-methylurea
23: N-{1-[N-(cis-4-aminocyclohexyl)-4-chloro-D-phenylalanyl]piperidin-4-yl}-N-cyclohexyl-2,5-dimethyl-2,5-dihydro-1H-pyrrole-1-carboxamide
33: N-{1-[N-(cis-4-aminocyclohexyl)-4-chloro-D-phenylalanyl]piperidin-4-yl})-N-cycloheptyl-N′,N′-diethylurea
35: N-{1-[N-(cis-4-aminocyclohexyl)-4-chloro-D-phenylalanyl]piperidin-4-yl}-N-cyclooctyl-N′,N′-diethylurea
38: N-{1-[N-(cis-4-aminocyclohexyl)-4-chloro-D-phenylalanyl]piperidin-4-yl}-N-cyclohexyl-N′-(2,2,2-trifluoroethyl)urea
48: N-{1-[N-(cis-4-aminocyclohexyl)-4-chloro-D-phenylalanyl]-3-methylpiperidin-4-yl}-N-cyclohexyl-N′,N′-diethylurea (trans)
50: N-{1-[N-(trans-4-aminocyclohexyl)-4-chloro-D-phenylalanyl]-3-methylpiperidin-4-yl}-N-cyclohexyl-N′,N′-diethylurea (trans)
67: N-{1-[N-(cis-4-aminocyclohexyl)-4-chloro-D-phenylalanyl]piperidin-4-yl}-N-cyclohexyl-N′-ethyl-N′-isopropylurea
74: N-(cis-4-{[(1R)-1-(4-chlorobenzyl)-2-(4-{cyclohexyl[(diethylamino)carbonyl]amino}piperidin-1-yl)-2-oxoethyl]amino}cyclohexyl)-2,2,2-trifluoroacetamide
N-(trans-4-{[(1R)-1-(4-chlorobenzyl)-2-(4-{cyclohexyl[(diethylamino)carbonyl]amino}piperidin-1-yl)-2-oxoethyl]amino}cyclohexyl)-2,2,2-trifluoroacetamide
75: N-{1-[N-[(1-benzoylpiperidin-4-yl)-4-chloro-D-phenylalanyl]piperidin-4-yl]-N-cyclohexyl-N′,N′-diethylurea
76: N-[1-[N-(trans-4-aminocyclohexyl)-4-chloro-D-phenylalanyl]piperidin-4-yl}-N-cyclohexyl-N′,N′-bis(2-fluoroethyl)urea
81: (2R,5S)-N-[1-[N-(cis-4-aminocyclohexyl)-4-chloro-D-phenylalanyl]piperidin-4-yl]-N-cyclohexyl-2,5-dimethylpyrrolidine-1-carboxamide
82: (2R,5S)-N-(1-{4-chloro-N-[cis-4-(dimethylamino)cyclohexyl]-D-phenylalanyl]piperidin-4-yl)-N-cyclohexyl-2,5-dimethylpyrrolidine-1-carboxamide
83: 4-{[(1R)-1-(4-chlorobenzyl)-2-(4-{cyclohexyl[(dimethylamino)carbonyl]amino}piperidin-1-yl)-2-oxoethyl]amino}-N,N-dimethylpiperidine-1-carboxamide
84: 4-{[(1R)-1-(4-chlorobenzyl)-2-(4-{cyclohexyl[(dimethylamino)carbonyl]amino}piperidin-1-yl)-2-oxoethyl]amino}-N,N-diethylpiperidine-1-carboxamide
85: N-(1-{4-chloro-N-[1-(pyrrolidin-1-ylcarbonyl)piperidin-4-yl]-D-phenylalanyl}piperidin-4-yl)-N-cyclohexyl-N′,N′-dimethylurea
86: N-(1-{4-chloro-N-[1-(piperidin-1-ylcarbonyl)piperidin-4-yl]-D-phenylalanyl}piperidin-4-yl)-N-cyclohexyl-N′,N′-dimethylurea
87: N-(1-{4-chloro-N-[1-(morpholin-4-ylcarbonyl)piperidin-4-yl]-D-phenylalanyl}piperidin-4-yl)-N-cyclohexyl-N′,N′-dimethylurea
88: 4-{[(1R)-1-(4-chlorobenzyl)-2-(4-{cyclohexyl[(dimethylamino)carbonyl]amino}piperidin-1-yl)-2-oxoethyl]amino}-N-phenylpiperidine-1-carboxamide
89: 4-{[(1R)-1-(4-chlorobenzyl)-2-(4-{cyclohexyl[(dimethylamino)carbonyl]amino}piperidin-1-yl)-2-oxoethyl]amino}-N-methyl-N-phenylpiperidine-1-carboxamide
90: N-benzyl-4-{[(1R)-1-(4-chlorobenzyl)-2-(4-cyclohexyl[(dimethylamino)carbonyl]amino}piperidin-1-yl)-2-oxoethyl]amino}-N-methylpiperidine-1-carboxamide
91: N-(1-{4-chloro-N-[1-(trifluoroacetyl)-piperidin-4-yl]-D-phenylalanyl}piperidin-4-yl)-N-cyclohexyl-N′,N′-dimethylurea
92: N-{1-[1N-(1-acetylpiperidin-4-yl)-4-chloro-D-phenylalanyl]piperidin-4-yl}-N-cyclohexyl-N′,N′-dimethylurea
93: N-{1-[4-chloro-N-(cis-4-hydroxy-4-phenylcyclohexyl)-D-phenylalanyl]piperidin-4-yl}-N-cyclohexyl-N′,N′-dimethylurea
N-{1-[4-chloro-N-(trans-4-hydroxy-4-phenylcyclohexyl)-D-phenylalanyl]piperidin-4-yl}-N-cyclohexyl-N′,N′-dimethylurea
94: N-(cis-4-{[(1R)-1-(4-chlorobenzyl)-2-(4-{cyclohexyl[(dimethylamino)carbonyl]amino}piperidin-1-yl)-2-oxoethyl]amino}cyclohexyl)-2,2,2-trifluoroacetamide
N-(trans-4-{([(1R)-1-(4-chlorobenzyl)-2-(4-{cyclohexyl[(dimethylamino)carbonyl]amino}piperidin-1-yl)-2-oxoethyl]amino}cyclohexyl)-2,2,2-trifluoroacetamide
95: N-[1-(4-chloro-N-{cis-4-[(4-fluorophenyl)amino]cyclohexyl}-D-phenylalanyl)-piperidin-4-yl]-N-cyclohexyl-N′,N′-dimethylurea
N-[1-(4-chloro-N-{trans-4-[(4-fluorophenyl)-amino]cyclohexyl}-D-phenylalanyl)piperidin-4-yl]-N-cyclohexyl-N′,N′-dimethylurea
98: N-[1-(4-chloro-N-{cis-4-[(2-hydroxyphenyl)amino]cyclohexyl}-D-phenylalanyl)-piperidin-4-yl]-N-cyclohexyl-N′,N′-dimethylurea
N-[1-(4-chloro-N-{trans-4-[(2-hydroxyphenyl)amino]cyclohexyl}-D-phenylalanyl)-piperidin-4-yl]-N-cyclohexyl-N′,N′-dimethylurea
102: N-[1-[4-chloro-N-(4-methoxycyclohexyl)-D-phenylalanyl]-3-methylpiperidin-4-yl]-N-cyclohexyl-N′,N′-dimethylurea (trans)
103: N-{1-[4-chloro-N-(4-phenylcyclohexyl)-D-phenylalanyl]-3-methylpiperidin-4-yl}-N-cyclohexyl-N′,N′-dimethylurea (trans)
104: N-{1-[N-(1-acetylpiperidin-4-yl)-4-chloro-D-phenylalanyl]-3-methylpiperidin-4-yl}-N-cyclohexyl-N′,N′-dimethylurea (trans)
105: N-{4-([(1R)-1-(4-chlorobenzyl)-2-(4-{cyclohexyl[(dimethylamino)carbonyl]amino}-3-methylpiperidin-1-yl)-2-oxoethyl]amino}cyclohexyl)-acetamide (trans)
106: N-(1-{4-chloro-N-[1-(trifluoroacetyl)-piperidin-4-yl]-D-phenylalanyl}-3-methylpiperidin-4-yl)-N-cyclohexyl-N′,N′-dimethylurea (trans)
107: N-(4-{[(1R)-1-(4-chlorobenzyl)-2-(4-{cyclohexyl[(dimethylamino)carbonyl]amino}-3-methylpiperidin-1-yl)-2-oxoethyl]amino}cyclohexyl)-2,2,2-trifluoroacetamide (trans)
108: N-{1-[N-(1-benzoylpiperidin-4-yl)-4-chloro-D-phenylalanyl]-3-methylpiperidin-4-yl}-N-cyclohexyl-N′,N′-dimethylurea
109: N-(1-{4-chloro-N-[1-(methylsulphonyl)-piperidin-4-yl]-D-phenylalanyl}-3-methylpiperidin-4-yl)-N-cyclohexyl-N′,N′-dimethylurea (trans)
110: N-{1-[4-chloro-N-(4-hydroxy-4-phenylcyclohexyl)-D-phenylalanyl]-3-methylpiperidin-4-yl}-N-cyclohexyl-N′,N′-dimethylurea (trans)
111: N-[1-(4-chloro-N-{4-[(4-fluorophenyl)-amino]cyclohexyl}-D-phenylalanyl)-3-methylpiperidin-4-yl]-N-cyclohexyl-N′,N′-dimethylurea
113: N-{1-[N-(cis-4-aminocyclohexyl)-4-chloro-D-phenylalanyl]-3-methylpiperidin-4-yl}-N-cyclohexyl-N′,N′-dimethylurea (trans)
115: N-[1-(4-chloro-N-{cis-4-[(2-methoxyphenyl)amino]cyclohexyl}-D-phenylalanyl)-piperidin-4-yl]-N-cyclohexyl-N′,N′-dimethylurea
N-[1-(4-chloro-N-{trans-4-[(2-methoxyphenyl)amino]cyclohexyl}-D-phenylalanyl)-piperidin-4-yl]-N-cyclohexyl-N′,N′-dimethylurea
116: N-(cis-4-{[(1R)-1-(4-chlorobenzyl)-2-(4-{cyclohexyl[(dimethylamino)carbonyl]amino}piperidin-1-yl)-2-oxoethyl]amino}cyclohexyl)acetamide
N-(trans-4-{[(1R)-1-(4-chlorobenzyl)-2-(4-{cyclohexyl[(dimethylamino)carbonyl]amino}piperidin-1-yl)-2-oxoethyl]amino}cyclohexyl)acetamide
117: N-(1-{4-chloro-N-[cis-4-(4-hydroxy-phenyl)cyclohexyl]-D-phenylalanyl}-3-methylpiperidin-4-yl)-N-cyclohexyl-N′,N′-dimethylurea
N-(1-{4-chloro-N-[trans-4-(4-hydroxyphenyl)-cyclohexyl]-D-phenylalanyl}-3-methylpiperidin-4-yl)-N-cyclohexyl-N′,N′-dimethylurea
118: N-(1-{4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea (trans)
119: N-{1-[4-chloro-N-(1-isonicotinoyl-piperidin-4-yl)-D-phenylalanyl]-3-methylpiperidin-4-yl}-N-cyclohexyl-N′,N′-dimethylurea (trans)
120: N-(1-{4-chloro-N-[cis-4-(1,3-dihydro-2H-isoindol-2-yl)cyclohexyl]-D-phenylalanyl}-3-methyl-piperidin-4-yl)-N-cyclohexyl-N′,N′-dimethylurea (trans)
121: N-{1-[4-chloro-N-(2-phenylpiperidin-4-yl)-D-phenylalanyl]-3-methylpiperidin-4-yl}-N-cyclohexyl-N′,N′-dimethylurea (trans)
122: N-(1-{4-chloro-N-[4-(3-oxopiperazin-1-yl)cyclohexyl]-D-phenylalanyl}-3-methylpiperidin-4-yl)-N-cyclohexyl-N′,N′-dimethylurea (trans).
Among the preferred compounds of formula I in which X is CR₆R₇, mention may be made of those having the following names:
2: N-{1-[N-(4-aminocyclohexyl)-4-chloro-D-phenylalanyl]piperidin-4-yl}-N-cyclohexyl-N′,N′-diethylurea
8: N-{1-[N-(cis-4-aminocyclohexyl)-4-chloro-D-phenylalanyl]piperidin-4-yl}-N-cyclohexyl-1,3-dihydro-2H-isoindole-2-carboxamide
9: N-{1-[N-(cis-4-aminocyclohexyl)-4-chloro-D-phenylalanyl]piperidin-4-yl}-N-cyclohexyl-2,5-dimethylpyrrolidine-1-carboxamide
12: N-{1-[N-(cis-4-aminocyclohexyl)-4-chloro-D-phenylalanyl]piperidin-4-yl}-N-cyclohexyl-N′,N′-dimethylurea
14: N-{1-[N-(cis-4-aminocyclohexyl)-4-chloro-D-phenylalanyl]piperidin-4-yl}-N-cyclohexyl-N′-methoxy-N′-methylurea
23: N-{1-[N-(cis-4-aminocyclohexyl)-4-chloro-D-phenylalanyl]piperidin-4-yl}-N-cyclohexyl-2,5-dimethyl-2,5-dihydro-1H-pyrrole-1-carboxamide
29: N-{1-[N-(cis-4-aminocyclohexyl)-4-chloro-D-phenylalanyl]piperidin-4-yl}-N-cyclobutyl-N′,N′-diethylurea
32: N-{1-[N-(cis-4-aminocyclohexyl)-4-chloro-D-phenylalanyl]piperidin-4-yl}-N-cyclopentyl-N′,N′-diethylurea
33: N-{1-[N-(cis-4-aminocyclohexyl)-4-chloro-D-phenylalanyl]piperidin-4-yl}-N-cycloheptyl-N′,N′-diethylurea
35: N-{1-[N-(cis-4-aminocyclohexyl)-4-chloro-D-phenylalanyl]piperidin-4-yl}-N-cyclooctyl-N′,N′-diethylurea
37: N-{1-[N-(cis-4-aminocyclohexyl)-4-chloro-D-phenylalanyl]piperidin-4-yl}-N′,N′-diethyl-N-phenylurea
38: N-{1-[N-(cis-4-aminocyclohexyl)-4-chloro-D-phenylalanyl]piperidin-4-yl}-N-cyclohexyl-N′-(2,2,2-trifluoroethyl)urea
45: N-{1-[4-chloro-N-(4-hydroxycyclohexyl)-D-phenylalanyl]piperidin-4-yl}-1-N-cyclohexyl-N′,N′-diethylurea
48: N-{1-[N-(cis-4-aminocyclohexyl)-4-chloro-D-phenylalanyl]-3-methylpiperidin-4-yl}-N-cyclohexyl-N′,N′-diethylurea (trans)
49: N-{1-[N-(cis-4-aminocyclohexyl)-4-chloro-D-phenylalanyl]-3-methylpiperidin-4-yl}-N-cyclohexyl-N′,N′-diethylurea (cis)
50: N-{1-[N-(trans-4-aminocyclohexyl)-4-chloro-D-phenylalanyl]-3-methylpiperidin-4-yl}-N-cyclohexyl-N′,N′-diethylurea (trans)
51: N-{1-[N-(trans-4-aminocyclohexyl)-4-chloro-D-phenylalanyl]-3-methylpiperidin-4-yl}-N-cyclohexyl-N′,N′-diethylurea (cis)
64: N-{1-[N-(cis-4-aminocyclohexyl)-4-chloro-D-phenylalanyl]piperidin-4-yl}-N′,N′-diethyl-N-(tetrahydro-2H-pyran-4-yl)urea
65: N-{1-[N-(cis-4-aminocyclohexyl)-4-chloro-D-phenylalanyl]piperidin-4-yl}-N′,N′-diethyl-N-piperidin-4-ylurea
67: N-{1-[N-(cis-4-aminocyclohexyl)-4-chloro-D-phenylalanyl]piperidin-4-yl}-N-cyclohexyl-N′-ethyl-N′-isopropylurea
71: N-{1-[N-(cis-4-aminocyclohexyl)-4-chloro-D-phenylalanyl]piperidin-4-yl}-N-cyclohexyl-2,2-dimethylhydrazinecarboxamide
74: N-(cis-4-{[(1R)-1-(4-chlorobenzyl)-2-(4-{cyclohexyl[(diethylamino)carbonyl]amino}piperidin-1-yl)-2-oxoethyl]amino}cyclohexyl)-2,2,2-trifluoroacetamide
N-(trans-4-{[(1R)-1-(4-chlorobenzyl)-2-(4-{cyclohexyl[(diethylamino)carbonyl]amino}piperidin-1-yl)-2-oxoethyl]amino}cyclohexyl)-2,2,2-trifluoroacetamide
76: N-{1-[N-(trans-4-aminocyclohexyl)-4-chloro-D-phenylalanyl]piperidin-4-yl}-N-cyclohexyl-N′,N′-bis(2-fluoroethyl)urea

Among the preferred compounds of formula I in which X is CR₆R₇, mention may also be made of those having the following names:
77: N-[(1R)-1-(4-chlorobenzyl)-2-(4-{cyclohexyl[2-(diethylamino)ethyl]amino}piperidin-1-yl)-2-oxoethyl]cyclohexane-1,4-diamine
79: N-(1-{4-chloro-N-[cis-4-(dimethylamino)-cyclohexyl]-D-phenylalanyl}piperidin-4-yl)-N-cyclohexyl-3,4-difluorobenzamide
80: N-(1-{4-chloro-N-[cis-4-(dimethylamino)-cyclohexyl]-D-phenylalanyl}piperidin-4-yl)-N-cycloheptyl-N′,N′-dimethylurea
81: (2R,5S)-N-{1-[N-(cis-4-aminocyclohexyl)-4-chloro-D-phenylalanyl]piperidin-4-yl}-N-cyclohexyl-2,5-dimethylpyrrolidine-1-carboxamide
82: (2R,5S)-N-(1-{4-chloro-N-[cis-4-(dimethylamino)cyclohexyl]-D-phenylalanyl}piperidin-4-yl)-N-cyclohexyl-2,5-dimethylpyrrolidine-1-carboxamide
93: N-{1-[4-chloro-N-(cis-4-hydroxy-4-phenylcyclohexyl)-D-phenylalanyl]piperidin-4-yl}-N-cyclohexyl-N′,N′-dimethylurea
N-{1-[4-chloro-N-(trans-4-hydroxy-4-phenylcyclohexyl)-D-phenylalanyl]piperidin-4-yl}-N-cyclohexyl-N′,N′-dimethylurea
94: N-(cis-4-{[(1R)-1-(4-chlorobenzyl)-2-(4-{cyclohexyl[(dimethylamino)carbonyl]amino}piperidin-1-yl)-2-oxoethyl]amino}cyclohexyl)-2,2,2-trifluoroacetamide
N-{trans-4-{[(1R)-1-(4-chlorobenzyl)-2-(4-(cyclohexyl[(dimethylamino)carbonyl]amino}piperidin-1-yl)-2-oxoethyl]amino}cyclohexyl)-2,2,2-trifluoroacetamide
95: N-[1-(4-chloro-N-{cis-4-[(4-fluorophenyl)amino]cyclohexyl}-D-phenylalanyl)-piperidin-4-yl]-N-cyclohexyl-N′,N′-dimethylurea
N-[1-[(4-chloro-N-{trans-4-(4-fluorophenyl)-amino]cyclohexyl}-D-phenylalanyl)piperidin-4-yl]-N-cyclohexyl-N′,N′-dimethylurea
96: N-{1-[4-chloro-N-(4-methoxycyclohexyl)-D-phenylalanyl]piperidin-4-yl}-N-cyclohexyl-N′,N′-dimethylurea
97: N-[1-(4-chloro-N-{cis-4-[(4-methoxyphenyl)amino]cyclohexyl}-D-phenylalanyl)-piperidin-4-yl]-N-cyclohexyl-N′,N′-dimethylurea
N-[1-(4-chloro-N-{trans-4-[(4-methoxyphenyl)-amino]cyclohexyl}-D-phenylalanyl)piperidin-4-yl]-N-cyclohexyl-N′,N′-dimethylurea
98: N-[1-(4-chloro-N-{cis-4-[(2-hydroxyphenyl)amino]cyclohexyl}-D-phenylalanyl)-piperidin-4-yl]-N-cyclohexyl-N′,N′-dimethylurea
N-[1-(4-chloro-N-{trans-4-[(2-hydroxyphenyl)amino]cyclohexyl}-D-phenylalanyl)-piperidin-4-yl]-N-cyclohexyl-N′,N′-dimethylurea
99: N-[1-(4-chloro-N-{4-[(dimethylamino)-methyl]-4-phenylcyclohexyl}-D-phenylalanyl)piperidin-4-yl]-N-cyclohexyl-N′,N′-dimethylurea
100: (2S,5S)-N-(1-{4-chloro-N-[cis-4-(dimethylamino)cyclohexyl]-D-phenylalanyl}piperidin-4-yl)-N-cyclohexyl-2,5-dimethylpyrrolidine-1-carboxamide
101: (2R,5R)-N-(1-{4-chloro-N-[cis-4-(dimethylamino)cyclohexyl]-D-phenylalanyl}piperidin-4-yl)-N-cyclohexyl-2,5-dimethylpyrrolidine-1-carboxamide
102: N-{1-[4-chloro-N-(4-methoxycyclohexyl)-D-phenylalanyl]-3-methylpiperidin-4-yl}-N-cyclohexyl-N′,N′-dimethylurea
103: N-{1-[4-chloro-N-(4-phenylcyclohexyl)-D-phenylalanyl]-3-methylpiperidin-4-yl]-N-cyclohexyl-N′,N′-dimethylurea
105: N-(4-{[(1R)-1-(4-chlorobenzyl)-2-(4-{cyclohexyl[(dimethylamino)carbonyl]amino}-3-methylpiperidin-1-yl)-2-oxoethyl]amino}cyclohexyl)-acetamide
107: N-(4-{[(1R)-1-(4-chlorobenzyl)-2-(4-{cyclohexyl[(dimethylamino)carbonyl]amino}-3-methylpiperidin-1-yl)-2-oxoethyl]amino}cyclohexyl)-2,2,2-trifluoroacetamide
110: N-{1-[4-chloro-N-(4-hydroxy-4-phenylcyclohexyl)-D-phenylalanyl]-3-methylpiperidin-4-yl}-1-N-cyclohexyl-N′,N′-dimethylurea
111: N-[1-(4-chloro-N-{4-[(4-fluorophenyl)-amino]cyclohexyl}-D-phenylalanyl)-3-methylpiperidin-4-yl]-N-cyclohexyl-N′,N′-dimethylurea
112: N-(1-[4-chloro-N-[cis-4-(dimethylamino)-cyclohexyl]-D-phenylalanyl}piperidin-4-yl)-N-cyclohexyl-N′,N′-dimethylurea
113: N-{1-[N-(cis-4-aminocyclohexyl)-4-chloro-D-phenylalanyl]-3-methylpiperidin-4-yl}-N-cyclohexyl-N′,N′-dimethylurea
115: N-[1-(4-chloro-N-{cis-4-[(2-methoxyphenyl)amino]cyclohexyl}-D-phenylalanyl)piperidin-4-yl]-N-cyclohexyl-N′,N′-dimethylurea
N-[1-(4-chloro-N-{cis-4-[(2-methoxyphenyl)-amino]cyclohexyl}-D-phenylalanyl)piperidin-4-yl]-N-cyclohexyl-N′,N′-dimethylurea
116: N-(cis-4-{[(1R)-1-(4-chlorobenzyl)-2-(4-{cyclohexyl[(dimethylamino)carbonyl]amino}piperidin-1-yl)-2-oxoethyl]amino}cyclohexyl)acetamide
N-(trans-4-{[(1R)-1-(4-chlorobenzyl)-2-(4-{cyclohexyl[(dimethylamino)carbonyl]amino}piperidin-1-yl)-2-oxoethyl]amino}cyclohexyl)acetamide
117: N-(1-{4-chloro-N-[cis-4-(4-hydroxyphenyl)cyclohexyl]-D-phenylalanyl}-3-methylpiperidin-4-yl)-N-cyclohexyl-N′,N′-dimethylurea
N-(1-{4-chloro-N-[trans-4-(4-hydroxyphenyl)-cyclohexyl]-D-phenylalanyl}-3-methylpiperidin-4-yl)-N-cyclohexyl-N′,N′-dimethylurea
118: N-(1-{4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea
120: N-(1-{4-chloro-N-[cis-4-(1,3-dihydro-2H-isoindol-2-yl)cyclohexyl]-D-phenylalanyl}-3-methylpiperidin-4-yl)-N-cyclohexyl-N′,N′-dimethylurea
122: N-(1-{4-chloro-N-[4-(3-oxopiperazin-1-yl)cyclohexyl]-D-phenylalanyl]-3-methylpiperidin-4-yl)-N-cyclohexyl-N′, N′-dimethylurea.

Among the preferred compounds of formula I in which X is NR₁₀, mention may be made of those having the following names:
4: N-[1-(N-8-azabicyclo[3.2.1]oct-3-yl-4-chloro-D-phenylalanyl)piperidin-4-yl]-N-cyclohexyl-N′,N′-diethylurea
5: N-[1-(N-1-azabicyclo[2.2.2]oct-3-yl-4-chloro-D-phenylalanyl)piperidin-4-yl]-N-cyclohexyl-N′,N′-diethylurea
30: N-[1-(N-8-azabicyclo[3.2.1]oct-3-yl-4-chloro-D-phenylalanyl)piperidin-4-yl]-N-cyclobutyl-N′,N′-diethylurea
75: N-{1-[N-[(1-benzoylpiperidin-4-yl)-4-chloro-D-phenylalanyl]piperidin-4-yl}-N-cyclohexyl-N′,N′-diethylurea.

Among the preferred compounds of formula I in which X is NR₁₀ mention may be made of those having the following names:
83: 4-{[(1R)-1-(4-chlorobenzyl)-2-(4-{(cyclohexyl[(dimethylamino)carbonyl]amino]piperidin-1-yl)-2-oxoethyl]amino}-N,N-dimethylpiperidine-1-carboxamide
84: 4-{[(1R)-1-(4-chlorobenzyl)-2-(4-{cyclohexyl[(dimethylamino)carbonyl]amino}piperidin-1-yl)-2-oxoethyl]amino}-N,N-diethylpiperidine-1-carboxamide
85: N-(1-{4-chloro-N-[1-(pyrrolidin-1-ylcarbonyl)piperidin-4-yl]-D-phenylalanyl}piperidin-4-yl)-N-cyclohexyl-N′,N′-dimethylurea
86: N-(1-{4-chloro-N-[1-(piperidin-1-ylcarbonyl)piperidin-4-yl]-D-phenylalanyl}piperidin-4-yl)-N-cyclohexyl-N′,N′-dimethylurea
87: N-(1-{4-chloro-N-[1-(morpholin-4-ylcarbonyl)piperidin-4-yl]-D-phenylalanyl}piperidin-4-yl)-N-cyclohexyl-N′,N′-dimethylurea
88: 4-{[(1R)-1-(4-chlorobenzyl)-2-(4-{(cyclohexyl[(dimethylamino)carbonyl]amino]piperidin-1-yl)-2-oxoethyl]amino}-N-phenylpiperidine-1-carboxamide
89: 4-{[(1R)-1-(4-chlorobenzyl)-2-(4-{cyclohexyl[(dimethylamino)carbonyl]amino}piperidin-1-yl)-2-oxoethyl]amino}-N-methyl-N-phenylpiperidine-1-carboxamide
90: N-benzyl-4-{[(1R)-1-(4-chlorobenzyl)-2-(4-{cyclohexyl[(dimethylamino)carbonyl]amino}piperidin-1-yl)-2-oxoethyl]amino)-N-methylpiperidine-1-carboxamide
91: N-(1-[4-chloro-N-[1-(trifluoroacetyl)-piperidin-4-yl]-D-phenylalanyl}piperidin-4-yl)-N-cyclohexyl-N′,N′-dimethylurea
92: N-{1-[N-(1-acetylpiperidin-4-yl)-4-chloro-D-phenylalanyl]piperidin-4-yl}-N-cyclohexyl-N′,N′-dimethylurea
104: N-{1-[N-[(1-acetylpiperidin-4-yl)-4-chloro-D-phenylalanyl]-3-methylpiperidin-4-yl}-N-cyclohexyl-N′,N′-dimethylurea
106: N-(1-{4-chloro-N-[1-(trifluoroacetyl)-piperidin-4-yl]-D-phenylalanyl}-3-methylpiperidin-4-yl)-N-cyclohexyl-N′,N′-dimethylurea
108: N-{1-[N-(1-benzoylpiperidin-4-yl)-4-chloro-D-phenylalanyl]-3-methylpiperidin-4-yl}-N-cyclohexyl-N′,N′-dimethylurea
109: N-(1-{4-chloro-N-[1-(methylsulphonyl)-piperidin-4-yl]-D-phenylalanyl}-3-methylpiperidin-4-yl)-N-cyclohexyl-N′,N′-dimethylurea
114: N-(1-{4-chloro-N-[1-(methylsulphonyl)-piperidin-4-yl]-D-phenylalanyl}piperidin-4-yl)-N-cyclohexyl-N′,N′-dimethylurea
119: N-{1-[4-chloro-N-(1-isonicotinoyl-piperidin-4-yl)-D-phenylalanyl]-3-methylpiperidin-4-yl}-N-cyclohexyl-N′,N′-dimethylurea
121: N-{1-[4-chloro-N-(2-phenylpiperidin-4-yl}-D-phenylalanyl]-3-methylpiperidin-4-yl)-N-cyclohexyl-N′,N′-dimethylurea.

According to another subject, the invention relates to a medicament, characterized in that it comprises a compound of formula (I) as described above, or an addition salt of this compound with a pharmaceutically acceptable acid, or else a hydrate or a solvate of the compound of formula (I).

According to another subject, the invention relates to a pharmaceutical composition, characterized in that it comprises a compound of formula (I) as described above, or a pharmaceutically acceptable salt, a hydrate or a solvate of this compound, and also at least one pharmaceutically acceptable excipient.

According to another subject, the invention relates to the use of a compound of formula (I) in the manufacture of a medicament for use in the treatment and prevention of obesity, diabetes and sexual dysfunctions that may affect both sexes, in particular erectile dysfunctions, in the treatment of cardiovascular diseases, and also in anti-inflammatory uses or in the treatment of alcohol dependency.

According to another subject, the invention relates to a method for preparing a compound of formula (I) as described above, characterized in that a reductive amination of a compound of formula (V):

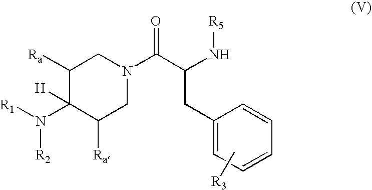

is carried out in the presence of a derivative of the group R₄ of ketone type, R₁, R₂, R₃, R₄, R₅, Rₐ and R_{a′}, being as defined in any one of claims 1 to 23.

In the subsequent text, the term “protective group (Pg)” is intended to mean a group that makes it possible, firstly, to protect a reactive function such as a hydroxyl or an amine during a synthesis and, secondly, to regenerate the intact reactive function at the end of synthesis. Examples of protective groups and also of methods of protection and of deprotection are given in “Protective Groups in Organic Synthesis”, Green W. et al., 1999, 3^{rd} Edition (John Wiley & Sons, Inc., New York).

In the subsequent text, the term “leaving group (Lg)” is intended to mean a group that can be readily cleaved from a molecule by heterolytic bond breaking, resulting in a pair of electrons leaving. This group can thus be readily replaced with another group in a substitution reaction, for example. Such leaving groups are, for example, halogens or an activated hydroxyl group such as a mesyl, tosyl, triflate, acetyl, etc. Examples of leaving groups and also references for the preparation thereof are given in “March's Advanced Organic Chemistry”, J. March et al., 5^{th} Edition, 2001, EMInter publisher.

The term “Boc group” is intended to mean a t-butoxycarbonyl group, “Bn group” is intended to mean a benzyl group, “CBz group” is intended to mean a benzyloxycarbonyl group, “Fmoc group” is intended to mean a 9-fluorenylmethylcarbamate group, and the term “h” is intended to mean hours.

According to another subject, the invention relates to the compounds of formulae (VI), (XVIII) and (XIX), in which R₁, R₂, R₃, R₄, R₅, Rₐ and R_{a′} are as defined above in the text and Pg represents a protective group:

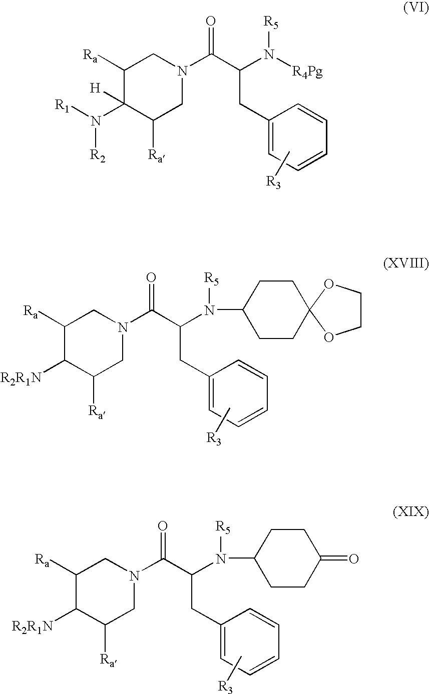

In accordance with the invention, the compounds of general formula (I) can be prepared according to the method presented in scheme 1.

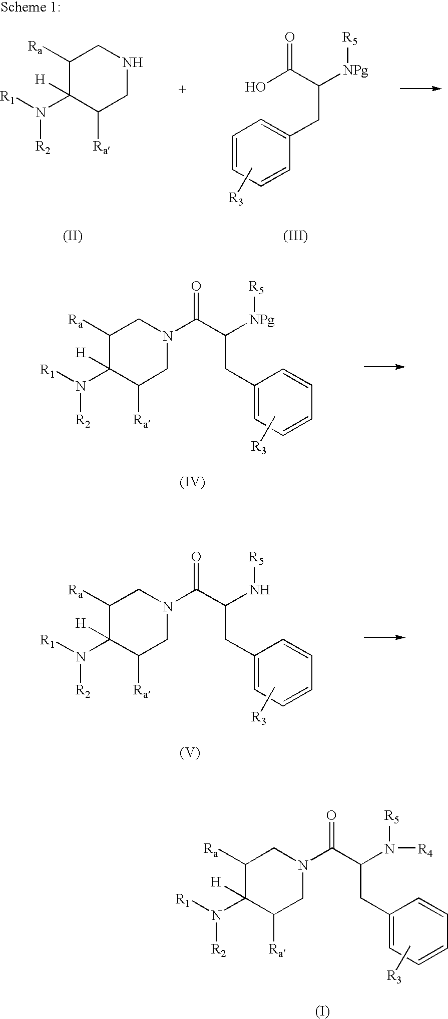

According to scheme 1, the compounds of formula (IV) can be prepared by coupling between the intermediates of formula (II) and an amino acid of formula (III), the amine function of which is protected with a protective group Pg (for example, a Boc, CBz or Fmoc group), under conventional peptide coupling conditions, using, for example, as coupling agent, dicyclocarbodiimide, 1-(3-dimethylaminopropyl)-3-ethyl-carbodiimide hydrochloride or bromotrispyrrolidino-phosphonium hexafluorophosphate, possibly in the presence of hydroxybenzotriazole, and, as base, triethylamine or diisopropylethylamine in a solvent such as dioxane, dichloromethane or acetonitrile.

The amino acids of general formula (III) are commercially available or can be prepared by methods described in the literature (Williams, R. M., Synthesis of Optically Active □-Aminoacids, Pergamon Press, Oxford, 1989).

The compounds of formula (V) are obtained by deprotection of the amine function of the compounds of formula (IV), by methods chosen from those known to those skilled in the art. They comprise, inter alia, the use of trifluoroacetic acid or hydrochloric acid in dichloromethane, dioxane, tetrahydrofuran or diethyl ether in the case of a protection with a Boc group, hydrogenation with the appropriate metal in methanol or ethanol in the case of a CBz, and of piperidine for an Fmoc group, at temperatures ranging from −10° C. to 100° C.

In a final step, the compounds of formula (I) are obtained by reductive amination, carried out by bringing the compounds of formula (V) into contact with a derivative of the group R₄ of ketone type, using a reducing agent such as sodium borohydride, sodium triacetoxyborohydride or sodium cyanoborohydride, possibly in the presence of a Brønsted acid (such as hydrochloric acid) or a Lewis acid (such as titanium tetraisopropoxide) in a solvent such as dichloroethane, dichloromethane, acetic acid or methanol, at temperatures of between −10° C. and 30° C.

The derivatives of the group R₄ of ketone type may be commercial or may be obtained by methods known to those skilled in the art, for example by acylation of the free hydroxyl or amine function of the derivative of ketone type

The compounds of general formula (I) can also be prepared according to the method presented in scheme 2.

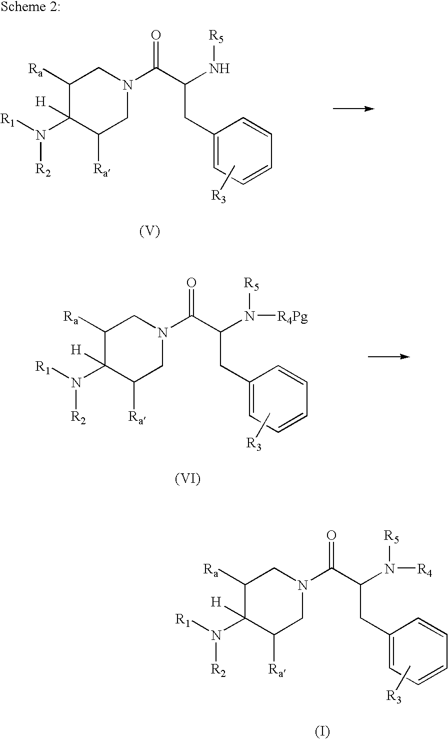

According to scheme 2, the compounds of formula (V), obtained as described above in scheme 1, are brought into contact with a derivative of the group R₄ of ketone type (reductive amination reaction, as described above in relation to scheme 1), said group R₁ bearing an amine-protecting group Pg, to give the compounds of formula (VI). The amine function of the compounds of formula (VI) is then deprotected by methods known to those skilled in the art, as described above.

Alternatively, the compounds of formula (VI) that give the compounds of formula (I) can be prepared according to the method presented in scheme 3.

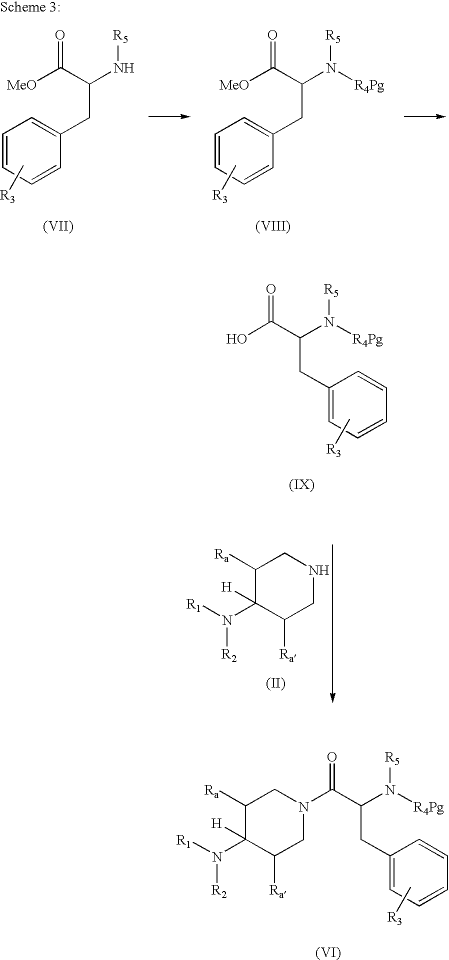

According to scheme 3, the compounds of formula (VIII) can be obtained by reductive amination, as described above, carried out using the amino acids of formula (VII). The amino acid of formula (VII) is commercially available when R₅═H, or it can be prepared by methods described in the literature (Williams, R. M., Synthesis of Optically Active □-Aminoacids, Pergamon Press, Oxford, 1989). When R₅ represents an alkyl group, the amino acids of formula (VII) can be prepared by alkylation of the commercial amino acid protected on the amine function, according to the alkylation methods known to those skilled in the art.

The compounds of formula (IX) can be synthesized by saponification of the esters of formula (VIII), for example in the presence of sodium hydroxide or of lithium hydroxide in a solvent such as methanol, tetrahydrofuran or water, or a mixture of these solvents.

The compounds of general formula (VI) can be prepared by peptide coupling between the intermediates of formula (II) and the amino acid of formula (IX), under peptide coupling conditions as described in scheme 1.

The compounds of formula (II) can be obtained according to the method presented in scheme 4.

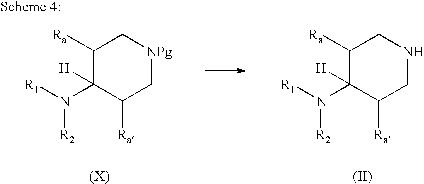

According to scheme 4, the compounds of formula (II) can be prepared from the compound of formula (X) (where Pg is an amine-protecting group as defined in scheme 1), after deprotection of the amine function by methods chosen from those known to those skilled in the art, as described above.

The compound of formula (X) is prepared according to the methods described in the literature or known to those skilled in the art, adapted according to the nature of the groups R₁ and R₂. Schemes 5 to 9 below present examples of preparation of the compounds of formula (X) according to various natures for the group R₂. For example, when R₂ represents a group —CO—R₁₅, where R₁₅ is as defined above, the preparation of the corresponding compound (Xa) can be carried out according to scheme 5.

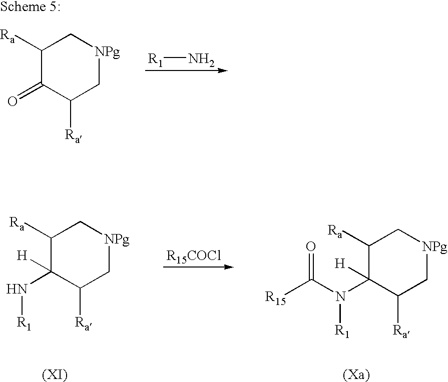

According to scheme 5, the compounds of formula (XI) can be obtained by reductive amination, under the conditions described above, of piperidone, the amine function of which is protected (for example, commercial Boc-piperidone). The compounds of formula (Xa) are then obtained by reaction of the compounds of formula (XI) with an acid chloride of formula R₁₅COCl, in the presence of an organic base such as triethylamine or pyridine, in a solvent such as dichloromethane or tetrahydrofuran.

A variant of scheme 5 consists in reacting a protected aminopiperidine (such as commercial 1-Boc-4-aminopiperidine) with an oxo derivative of the group R, under reductive amination conditions described above.

Scheme 6 presents a pathway for preparing the compounds of formula (Xb) and (Xc), which correspond respectively to the compounds of formula (X) in which R₂ represents a group —CO—NR₁₆R₁₇ and —CO—NR₁₅—NR₁₆R₁₇, where R₁₅, R₁₆ and R₁₇ are as defined above.

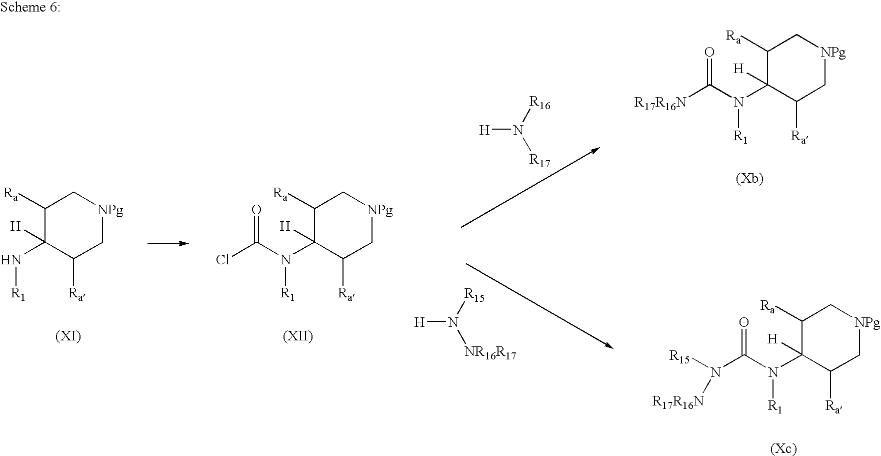

According to scheme 6, the compounds of formula (XII) can be prepared from the compounds of formula (XI) by reaction with phosgene, triphosgene or trichloromethyl chloroformate in dichloromethane or toluene in the presence of triethylamine or of pyridine and an amine at temperatures ranging from −10° C. to 80° C. Reaction of the compounds of formula (XII) with an amine of formula HN(R₁₆)(R₁₇) or a hydrazine of formula HN(R₁₅) (NR₁₆R₁₇) gives, respectively, the compounds of formulae (Xb) and (Xc).

Scheme 7 presents a pathway for preparing the compounds of formula (Xd), corresponding to the compounds of formula (X) in which R₂ represents a group —(CH₂)ₓ—NR₁₆R₁₇, where x=2, 3 or 4 and where R₁₆ and R₁₇ are as defined above.

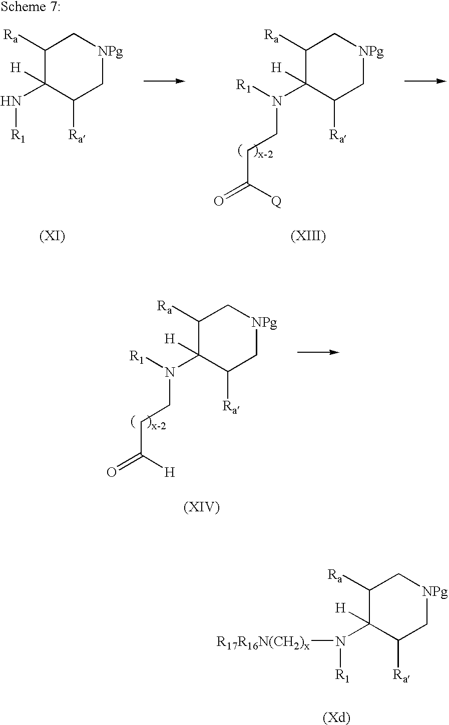

According to scheme 7, the compounds of formula (XIII) can be obtained by reductive amination carried out on the compounds of formula (XI) in the presence of an aldehyde of formula Q-CO—(CH₂)ₓ₋₂—CHO, where Q represents an —O-alkyl or —N(O-alkyl) (alkyl) group, using a reducing agent as described above in relation to scheme 1.

The compounds of general formula (XIII) can then be reduced to give the aldehydes of formula (XIV), using a reducing agent such as diisobutyl aluminium hydride or sodium aluminium tetrahydride when Q is an —O-alkyl group, or by reduction with lithium aluminium hydride when Q is an —N(O-alkyl)(alkyl) group (for example —N(OMe)Me), obtained, for example, by reaction of an organomagnesium compound, such as diisopropylmagnesium chloride, with the compounds of formula (XIII) where Q=-O-alkyl, in the presence of an alkylhydroxyalkyl amine such as N,O-dimethylhydroxylamine, in solvents such as tetrahydrofuran or diethyl ether at temperatures ranging from −78° C. to 20° C.

The compounds of formula (Xd) can then be prepared by reductive amination carried out in the presence of an amine of formula R₁₇R₁₆NH, using a reducing agent as described above.

Scheme 8 presents a pathway for preparing the compounds of formula (Xe), corresponding to the compounds of formula (X) in which R₂ represents a group —(CH₂)ₓ-aryl (where x=0, 1, 2, 3 or 4) or —(CH₂)ₓ—heteroaryl (where x=1, 2, 3 or 4).

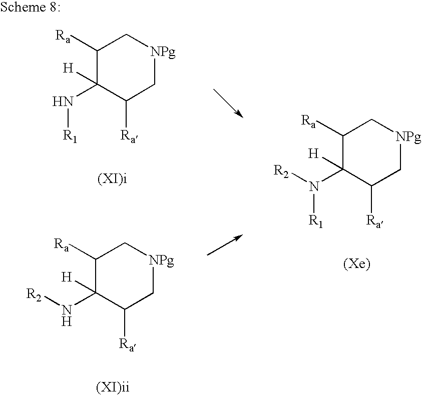

According to scheme 8, the compounds of formulae (Xe), in which R₂ represents a group —(CH₂)ₓ-heteroaryl (where x=1, 2, 3 or 4), can be obtained by reductive amination using the compounds of formula (XI)i, carried out in the presence of an aldehyde of formula: heteroaryl-(CH₂)ₓ₋₁—CHO, using a reducing agent as described above in relation to scheme 1.

The same reaction can also be used to obtain the compounds of formula (Xd), using an aldehyde of formula R₁₇R₁₆N—(CH₂)ₓ₋₁—CHO

The compounds of formulae (XI)ii, in which R₂ represents a group —(CH₂)ₓ-aryl (where x=0, 1, 2, 3 or 4), can be obtained by reductive amination using piperidone protected on the amine function, carried out in the presence of an amine of formula: aryl-(CH₂)ₓ—NH₂, using a reducing agent as described above in relation to scheme 1. The compounds of formulae (Xe) in which R₂ represent a group —(CH₂)ₓ-aryl can then be obtained by reductive amination using the compounds of formula (XI)ii, carried out in the presence of a derivative of the group R₁ of oxo type.

Scheme 9 gives the details of an alternative for synthesizing the compounds of formula (Xe) in which R₂ represents a group —(CH₂)ₓ-heteroaryl, where x is equal to 2 or 3.

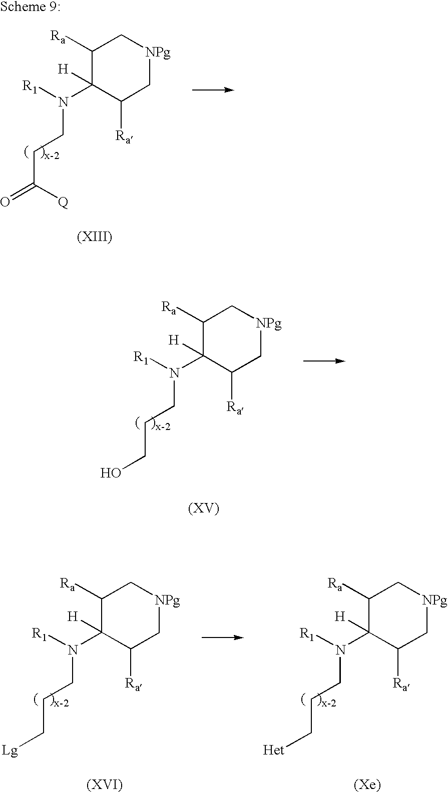

According to scheme 9, the compounds of formula (XIII), in which Q represents an —O-alkyl group, can be reduced to the corresponding alcohols using a reducing agent such as lithium aluminium hydride in a solvent such as diethyl ether or tetrahydrofuran, at temperatures ranging from −60° C. to 20° C.

The hydroxyl group of the compounds of formula (XV) is then converted to a leaving group (Lg), such as chloride or mesylate, for example by the action of tetrabromomethane and of triphenylphosphine in a solvent such as dichloromethane, or by the action of methanesulphonyl chloride in the presence of an organic base such as triethylamine at temperatures ranging from −20° C. to ambient temperature, to give the compounds of formula (XVI).

The compounds of formula (Xe) are then synthesized by means of a nucleophilic substitution reaction between the compounds of formula (XVI) and the anion of a heteroaryl (“Het” group).

According to a variant of scheme 1, when the compounds of formula (I) comprise, as group R₄, a group of formula (a) of cyclohexyl type, i.e. a group of formula (a) where p=2 and X=—C(R₆)(R₇)—, where R₆ represents a group —OR₈, R₇ and R₈ being as defined above, then the preparation of the compounds of formula (I) can be carried out as described in scheme 10.

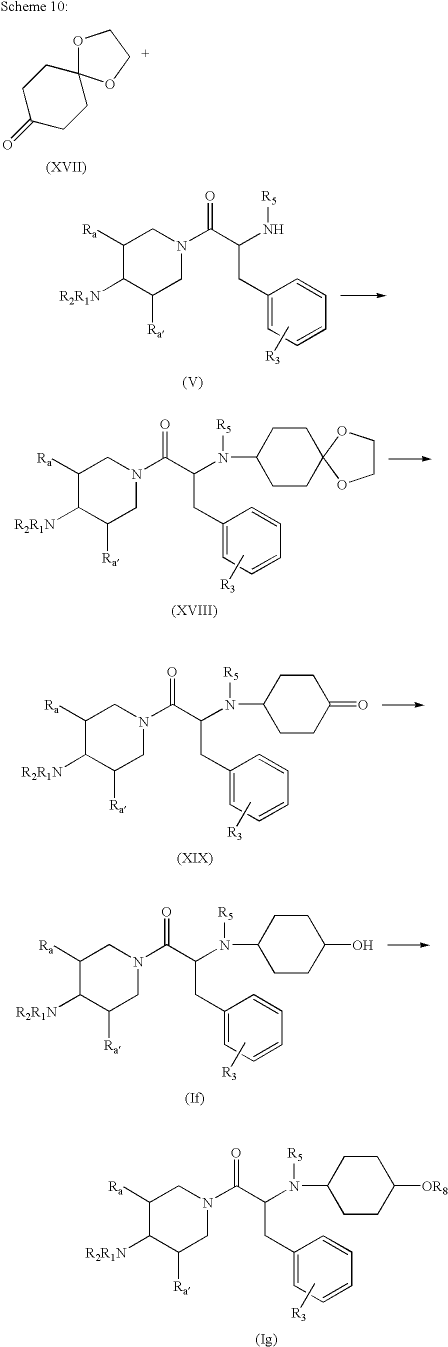

According to scheme 10, the compounds of formula (XVIII) can be obtained by a reductive amination between the commercial compound of formula (XVII) and the compounds of formula (V), under conditions as described in scheme 1.

Deprotection of the oxo function of the compound of formula (XVIII) in the presence of an acid such as hydrochloric acid or pyridinium tosylate in tetrahydrofuran or acetone, at temperatures of between 0° C. and 80° C., gives the compound of formula (XIX).

The compounds of formula (If) are prepared by reduction of the compounds of formula (XIX) under conditions as described in scheme 6.

When R₈ is different from a hydrogen atom, functionalization of the compounds of formula (If) is carried out, for example an alkylation in the presence of a base such as sodium hydride and of a derivative of the group R₈ comprising a leaving group Lg, which gives the compounds of formula (Ig).

In schemes 1 to 10, the starting compounds and the reactants, when the method for preparing them is not described, are commercially available or are described in the literature, or else can be prepared according to methods which are described therein or which are known to those skilled in the art.

A subject of the present invention is also the compounds of formulae (II), (IV), (V), (VI), (VIII), (IX), (X), (XVIII) and (XIX): these compounds are useful as synthesis intermediates for the compounds of formula (I).

The following examples describe the preparation of certain compounds in accordance with the invention. These examples are not limiting and merely illustrate the present invention. The numbers of the compounds exemplified refer to those given in the table hereinafter, which illustrates the chemical structures and the physical properties of some compounds according to the invention.

### EXAMPLE 1

### N-[1-(4-chloro-N-piperidin-4-yl-D-phenylalanyl)piperidin-4-yl]-N-cyclohexyl-N′,N′-diethylurea (compound No. 1)

1.1: tert-butyl 4-(cyclohexylamino)-piperidine-1-carboxylate

15.0 g of 1-Boc-piperidone are placed in 370 ml of dichloromethane under N₂ in the presence of 7.47 g of cyclohexylamine and of 20.7 g of sodium triacetoxyborohydride. The reaction medium is stirred at ambient temperature for 16 h. After the addition of 30 ml of methanol, 300 g of DOWEX® 50X2 resin are added and the mixture is stirred for 45 min. The resin is then filter-dried and washed with tetrahydrofuran and then methanol. The expected compound is then released with a 2N solution of aqueous ammonia in methanol. After concentration to dryness, 13.85 g of tert-butyl 4-(cyclohexylamino)piperidine-1-carboxylate are obtained, which product is subsequently used as it is.

1.2: tert-butyl 4-[{cyclohexyl[(diethylamino)-carbonyl]amino}piperidine-1-carboxylate

5.92 ml of diphosgene are placed in 150 ml of dichloromethane at 0° C. under N₂. A solution of 13.85 g of tert-butyl 4-(cyclohexylamino)piperidine-1-carboxylate and of 34.18 ml of triethylamine is added dropwise. The solution is stirred at 0° C. for 30 min and then at ambient temperature for 1 h. The reaction medium is again placed at 0° C., and 5.92 ml of diphosgene and 34.18 ml of triethylamine are again added. After stirring at ambient temperature for 1 h, 25.4 ml of diethylamine are added. The mixture is stirred at ambient temperature for 16 h. After evaporation of the dichloromethane, 200 ml of 0.5N hydrochloric acid are added. Extraction is carried out with dichloromethane until the aqueous phase is completely depleted. After drying over MgSO₄ and concentration to dryness, the residue obtained is chromatographed on silica gel, elution being carried out with a 98/2 and then 95/5 mixture of dichloromethane and methanol, to give 16.77 g of tert-butyl 4-{cyclohexyl[(diethylamino)carbonyl]amino}-piperidine-1-carboxylate.

1.3: N-cyclohexyl-N′,N′-diethyl-N-piperidin-4-ylurea

16.77 g of tert-butyl 4-(cyclohexyl[(diethyl-amino)carbonyl]amino}piperidine-1-carboxylate mixed with diethylurea are placed in 54.9 ml of 4N hydrochloric acid in dioxane. The reaction medium is stirred at ambient temperature for 16 h. After evaporation to dryness, 1N sodium hydroxide is added up to a pH of 10 and extraction is carried out with dichloromethane until the aqueous phase is completely depleted. After drying over MgSO₄ and evaporation to dryness, the crude is chromatographed on silica gel, elution being carried out with a 98/2/0.2, 95/5/0.5, then 9/1/0.1 and 5/5/0.5 mixture of dichloromethane, methanol and aqueous ammonia, so as to obtain 11.27 g of N-cyclohexyl-N′,N′-diethyl-N-piperidin-4-ylurea.

1.4: tert-butyl [(1R)-1-(4-chlorobenzyl)-2-(4-{cyclohexyl[(diethylamino)carbonyl]amino}piperidin-1-yl)-2-oxoethyl]carbamate

2.85 g of N-cyclohexyl-N′,N′-diethyl-N-piperidin-4-ylurea are dissolved in 101 ml of dichloromethane in the presence of 3.04 g of 4-chloro-D-Boc-phenylalanine, of 1.37 g of hydroxybenzotriazole, of 1.95 g of 1-(3-dimethylaminopropyl)-3-ethylcarbodiimide hydrochloride and of 1.77 ml of diisopropylethylamine. The mixture is stirred at ambient temperature for 16 h. After evaporation to dryness, the residue is hydrolysed and extraction is carried out with ethyl acetate until the aqueous phase is completely depleted. The organic phases are washed with a 1N sodium hydroxide solution and then with water. After drying over MgSO₄ and concentration to dryness, the crude is chromatographed on silica gel, elution being carried out with a 98/2 and then 95/5 mixture of dichloromethane and methanol, to give 5.04 g of tert-butyl [(1R)-1-(4-chlorobenzyl)-2-(4-(cyclohexyl[(diethylamino)carbonyl]amino}piperidin-1-yl)-2-oxoethyl]carbamate.

1.5: N-[1-(4-chloro-D-phenylalanyl)piperidin-4-yl]-N-cyclohexyl-N′,N′-diethylurea

5.16 g of tert-butyl [(1R)-1-(4-chlorobenzyl)-2-(4-{cyclohexyl[(diethylamino)carbonyl]-amino}piperidin-1-yl)-2-oxoethyl]carbamate are placed in 22.89 ml of 4N hydrochloric acid in dioxane. The reaction medium is stirred at ambient temperature for 24 h. After evaporation to dryness, the residue is taken up with ethyl acetate and washed with a saturated aqueous sodium hydrogen carbonate solution and then with a saturated aqueous sodium chloride solution. After drying over MgSO₄ and concentration to dryness, the crude is chromatographed on silica gel, elution being carried out with a 95/5 mixture of dichloromethane and methanol. 2.9 g of N-[1-(4-chloro-D-phenylalanyl)piperidin-4-yl]-N-cyclohexyl-N′,N′-diethylurea are obtained.

1.6: tert-butyl 4-{[(1R)-1-(4-chlorobenzyl)-2-(4-{cyclohexyl[(diethylamino)carbonyl]amino}piperidin-1-yl)-2-oxoethyl]amino}piperidine-1-carboxylate

0.5 g of N-[1-(4-chloro-D-phenylalanyl)-piperidin-4-yl]-N-cyclohexyl-N′,N′-diethylurea is dissolved in 5 ml of dichloromethane in the presence of 0.30 g of N-Boc-piperidone and of 0.42 g of sodium triacetoxyborohydride under N₂. Stirring is maintained at ambient temperature for 18 h. After hydrolysis, extraction is carried out with dichloromethane until the aqueous phase is completely depleted. The organic phase is washed with water. After drying over MgSO₄ and concentration to dryness, the crude obtained is chromatographed on silica gel, elution being carried out with a mixture of cyclohexane and ethyl acetate (9/1). 0.2 g of tert-butyl 4-{([(1R)-1-(4-chlorobenzyl)-2-(4-{cyclohexyl[(diethylamino)carbonyl]amino}-piperidin-1-yl)-2-oxoethyl]amino}piperidine-1-carboxylate is obtained.

1.7: N-[1-(4-chloro-N-piperidin-4-yl-D-phenylalanyl)piperidin-4-yl]-N-cyclohexyl-N′,N′-diethylurea

0.26 g of tert-butyl 4-{([(1R)-1-(4-chlorobenzyl)-2-(4-{cyclohexyl[(diethylamino)-carbonyl]amino)piperidin-1-yl)-2-oxoethyl]amino}-piperidine-1-carboxylate is dissolved in 2 ml of diethyl ether and then 2.74 ml of 2N hydrochloric acid in diethyl ether are added. The reaction medium is stirred at ambient temperature for 16 h. After partial concentration, the precipitate obtained is filter-dried, and is then triturated in a mixture of ethanol and dichloromethane. The crystals are filter-dried and rinsed with ethanol. The hydrochloride thus obtained is dried over P₂O₅ under reduced pressure. 0.18 g of expected N-[1-(4-chloro-N-piperidin-4-yl-D-phenylalanyl)piperidin-4-yl]-N-cyclohexyl-N′,N′-diethylurea is obtained in the form of a white solid.

Melting point>220° C.; M+H⁺=546; [α]_{D}²⁰=+7.0° (c=0.995 g/100 ml, DMSO).

¹H NMR (200 MHz, DMSO-d): 9.95-8.95 (exchangeable Hs), 7.39 (d, J=8 Hz, 2H), 7.20 (d, J=8 Hz, 2H), 4.78 (m, 1H), 4.29 (t, J=12 Hz, 1H), 3.32 (unresolved peak, 6H+ H₂O), 3.64-2.84 (m, 9H), 2.24-1.10 (m, 18H), 0.98 (t, J=6 Hz, 3H), 0.95 (t, J=6 Hz, 3H). Elemental analysis: exp % C, 52.51, % H, 7.86, % N, 10.15; th: % C, 57.88, % H, 8.65% N, 11.25

### EXAMPLE 2

### N-[1-(N-1-azabicyclo[2.2.2]oct-3-yl-4-chloro-D-phenylalanyl)piperidin-4-yl]-N-cyclohexyl-N′,N′-diethylurea hydrochloride (compound No. 5)

2.1: N-[1-(N-1-azabicyclo[2.2.2]oct-3-yl-4-chloro-D-phenylalanyl)piperidin-4-yl]-N-cyclohexyl-N′,N′-diethylurea

0.23 g of N-[1-(4-chloro-D-phenylalanyl)-piperidin-4-yl]-N-cyclohexyl-N′,N′-diethylurea, obtained in step 1.5 above, is dissolved in 3 ml of dichloromethane in the presence of 0.089 g of 3-quinuclidinone hydrochloride and of 0.22 g of sodium triacetoxyborohydride under N₂. Stirring is maintained at ambient temperature for 18 h. After the addition of 0.044 g of ketone and 0.222 g of triacetoxyborohydride, the reaction medium is stirred for 48 h. After the addition of 2 ml of methanol, the solution is loaded onto a cartridge containing 4 g of DOWEX® 50X2 resin. The resin is washed with THF, with water and then with methanol. The expected compound is then released with 2N aqueous ammonia in methanol. After concentration to dryness, 0.212 g of a mixture of (R,S) and (R,R) diastereoisomers of N-[1-(N-1-azabicyclo[2.2.2]oct-3-yl-4-chloro-D-phenylalanyl)piperidin-4-yl]-N-cyclohexyl-N′,N′-diethylurea is obtained.

2.2: (R,S) and (R,R) diastereoisomers of N-[1-[(N-1-azabicyclo[2.2.2]oct-3-yl-4-chloro-D-phenylalanyl)piperidin-4-yl]-N-cyclohexyl-N′,N′-diethylurea hydrochloride

0.21 g of (R,S) and (R,R) diastereoisomers of N-[1-(N-1-azabicyclo[2.2.2]oct-3-yl-4-chloro-D-phenylalanyl)piperidin-4-yl]-N-cyclohexyl-N′,N′-diethylurea is mixed with 0.37 ml of 2N hydrochloric acid in diethyl ether. The solution is triturated. The crystals obtained are rinsed with diethyl ether and filter-dried. 0.204 g of (R,S) and (R,R) diastereoisomers of N-[1-(N-1-azabicyclo[2.2.2]oct-3-yl-4-chloro-D-phenylalanyl)piperidin-4-yl]-N-cyclohexyl-N′,N′-diethylurea hydrochloride is obtained in the form of a white solid.

Melting point=169° C.; M+H⁺=572.

### EXAMPLE 3

### 1-[(2R)-3-(4-chlorophenyl)-2-(piperidin-4-ylamino)propanoyl]-N-cyclohexyl-N-{2-[methoxy(methyl)amino]ethyl}piperidin-4-amine (compound No. 16)

3.1: tert-butyl 4-[cyclohexyl(2-ethoxy-2-oxoethyl)amino]piperidine-1-carboxylate

4.5 g of tert-butyl 4-(cyclohexylamino)-piperidine-1-carboxylate are dissolved in 159 ml of dichloromethane in the presence of 4.88 g of ethyl glyoxilate and of 13.5 g of sodium triacetoxyborohydride under N₂. Stirring is maintained at ambient temperature for 18 h. After aqueous hydrolysis, extraction is carried out with dichloromethane until the aqueous phase is completely depleted. The organic phase is washed with a saturated aqueous sodium hydrogen carbonate solution and then with water. After drying over MgSO₄ and concentration to dryness, the crude obtained is chromatographed on silica gel, elution being carried out with a 99/1 mixture of dichloromethane and methanol. 3 g of tert-butyl 4-[cyclohexyl(2-ethoxy-2-oxoethyl)amino]-piperidine-1-carboxylate are obtained.

3.2: tert-butyl 4-(cyclohexyl{2-[methoxy-(methyl)amino]-2-oxoethyl}amino)piperidine-1-carboxylate

3.09 g of tert-butyl 4-[cyclohexyl(2-ethoxy-2-oxoethyl)amino]piperidine-1-carboxylate are dissolved in 84 ml of tetrahydrofuran under N₂, and the solution is cooled to −20° C. After the addition of 1.54 g of N,O-dimethylhydroxylamine hydrochloride, 20.96 ml of 2M isopropylmagnesium chloride in tetrahydrofuran are added such that the temperature does not exceed −10° C. After stirring for 1 h 30 min, a further 0.51 g of N,O-dimethylhydroxylamine hydrochloride and 4.2 ml of 2M isopropyl magnesium compound in tetrahydrofuran are added at −10° C. Stirring is maintained for 30 min. After evaporation of the tetrahydrofuran, the crude obtained is taken up with dichloromethane and hydrolysed. Extraction is carried out with dichloromethane until the aqueous phase is completely depleted. The organic phase is washed with water and then with a saturated aqueous sodium chloride solution. After drying over MgSO₄ and concentration to dryness, the crude obtained is chromatographed on silica gel, elution being carried out with a 95/5 mixture of dichloromethane and methanol. 1.11 g of tert-butyl 4-(cyclohexyl(2-[methoxy(methyl)amino]-2-oxoethyl}amino)piperidine-1-carboxylate are obtained.

3.3: tert-butyl 4-[cyclohexyl(2-oxoethyl)amino]piperidine-1-carboxylate

4.02 g of tert-butyl 4-(cyclohexyl(2-[methoxy(methyl)amino]-2-oxoethyl}amino)piperidine-1-carboxylate are dissolved in 105 ml of anhydrous diethyl ether under N₂ at −10° C. 12.6 ml of 1M lithium aluminium hydride in tetrahydrofuran are added. After stirring at 0° C. for 1 h, a saturated potassium sulphate solution is added up to a pH of 6-7. Extraction is carried out with dichloromethane until the aqueous phase is completely depleted. The organic phase is washed with water and then with a saturated aqueous sodium chloride solution. After drying over MgSO₄ and concentration to dryness, 3.39 g of tert-butyl 4-[cyclohexyl(2-oxoethyl)amino]piperidine-1-carboxylate are obtained, which product is subsequently used as it is.

3.4: tert-butyl 4-(cyclohexyl{2-[methoxy-(methyl)amino]ethyl}amino)piperidine-1-carboxylate

1.69 g of tert-butyl 4-[cyclohexyl(2-oxoethyl)amino]piperidine-1-carboxylate are dissolved in 52 ml of dichloromethane in the presence of 5.10 g of N,O-dimethylhydroxylamine hydrochloride and of 4.43 g of sodium triacetoxyborohydride under N₂. Stirring is maintained at ambient temperature for 5 days. After the addition of methanol and evaporation to dryness, extraction is carried out with dichloromethane until the aqueous phase is completely depleted. The organic phase is washed with a saturated aqueous sodium hydrogen carbonate solution, with water and then with a saturated aqueous sodium chloride solution. After drying over MgSO₄ and concentration to dryness, the crude obtained is chromatographed on silica gel, elution being carried out with a 98/2 mixture of dichloromethane and methanol. 1.03 g of tert-butyl 4-(cyclohexyl{2-[methoxy(methyl)amino]ethyl}amino)-piperidine-1-carboxylate are obtained.

3.5: N-cyclohexyl-N-{2-[methoxy(methyl)-amino]ethyl}piperidin-4-amine

1.033 g of tert-butyl 4-cyclohexyl{2-[methoxy(methyl)amino]ethyl}amino)piperidine-1-carboxylate are placed in 28 ml of diethyl ether, and 14 ml of 2N hydrochloric acid in diethyl ether are added. The reaction medium is stirred at ambient temperature for 16 h. After evaporation to dryness, the crude is taken up with dichloromethane and a saturated sodium hydrogen carbonate solution is added, and extraction is carried out with dichloromethane until the aqueous phase is completely depleted. After drying over MgSO₄ and evaporation to dryness, 0.18 g of N-cyclohexyl-N-{2-[methoxy(methyl)amino]ethyl}piperidin-4-amine is obtained.

3.6: Methyl N-[1-(tert-butoxycarbonyl)-piperidin-4-yl]-4-chloro-D-phenylalaninate

10 g of p-D-chlorophenylalanine methyl ester are dissolved in 248 ml of dichloromethane in the presence of 8.8 g of N-Boc-piperidone and of 14.4 g of sodium triacetoxyborohydride under N₂. Stirring is maintained at ambient temperature for 18 h. After the addition of methanol and evaporation to dryness, the crude is taken up with a saturated aqueous sodium hydrogen carbonate solution, and extraction is carried out with ethyl acetate until the aqueous phase is completely depleted. After drying over MgSO₄ and concentration to dryness, 15.87 g of methyl N-[1-(tert-butoxycarbonyl)piperidin-4-yl]-4-chloro-D-phenylalaninate are obtained.

3.7: N-[1-(tert-butoxycarbonyl)piperidin-4-yl]-4-chloro-D-phenylalanine

15.8 g of methyl N-[1-(tert-butoxycarbonyl)-piperidin-4-yl]-4-chloro-D-phenylalaninate are dissolved in 200 ml of a tetrahydrofuran/water (1/1) mixture, and 3.35 g of lithium hydroxide hydrate are added. Stirring is maintained at ambient temperature for 16 h. Potassium sulphate is added up to a pH of 7. The precipitate obtained is filter-dried and rinsed with diethyl ether. After drying over P₂O₅, 11.38 g of N-[1-(tert-butoxycarbonyl)piperidin-4-yl]-4-chloro-D-phenylalanine are obtained.

3.8: tert-butyl 4-({(1R)-1-(4-chlorobenzyl)-2-[4-(cyclohexyl{2-[methoxy(methyl)amino]ethyl}amino)-piperidin-1-yl]-2-oxoethyl}amino)piperidine-1-carboxylate

0.18 g of N-cyclohexyl-N-{2-[methoxy(methyl)-amino]ethyl}piperidin-4-amine, obtained in step 3.5, is dissolved in 6.8 ml of dichloromethane in the presence of 0.26 g of N-[1-(tert-butoxycarbonyl)piperidin-4-yl]-4-chloro-D-phenylalanine (obtained in step 3.7), of 0.092 g of hydroxybenzotriazole, of 0.13 g of 1-(3-dimethylaminopropyl)-3-ethylcarbodiimide hydrochloride and of 0.12 ml of diisopropylethylamine. The mixture is stirred at ambient temperature for 16 h. After hydrolysis, extraction is carried out with dichloromethane until the aqueous phase is completely depleted. The organic phase is washed with water and then with a saturated sodium chloride solution. After drying over MgSO₄ and concentration to dryness, the crude is chromatographed on silica gel, elution being carried out with a 98/2 and then 97/3 mixture of dichloromethane and methanol, to give 0.15 g of tert-butyl 4-({(1R)-1-(4-chlorobenzyl)-2-[4-(cyclohexyl-{2-[methoxy(methyl)amino]ethyl}amino)piperidin-1-yl]-2-oxoethyl}amino)piperidine-1-carboxylate.

3.9: 1-[(2R)-3-(4-chlorophenyl)-2-(piperidin-4-ylamino)propanoyl]-M-cyclohexyl-N-{2-[methoxy-(methyl)amino]ethyl}piperidin-4-amine hydrochloride

0.147 g of tert-butyl 4-({(1R)-1-(4-chlorobenzyl)-2-[4-(cyclohexyl{2-[methoxy(methyl)-amino]ethyl}amino)piperidin-1-yl]-2-oxoethyl]amino)-piperidine-1-carboxylate is placed in 2.3 ml of diethyl ether, and 0.58 ml of 2N hydrochloric acid in diethyl ether is added. The reaction medium is stirred at ambient temperature for 16 h. After evaporation to dryness and hydrolysis, extraction is carried out with dichloromethane until the aqueous phase is completely depleted. The organic phase is washed with a saturated sodium hydrogen carbonate solution, with water and then with a saturated aqueous sodium chloride solution. After drying over MgSO₄ and concentration to dryness, 2 ml of isopropanol and 2.43 ml of 0.1N hydrochloric acid in isopropanol are added. After concentration to dryness, the residue is taken up with diethyl ether and the solid is triturated. The crystals obtained are filter-dried and rinsed with diethyl ether. After drying over P₂O₅, 0.08 g of 1-[(2R)-3-(4-chlorophenyl)-2-(piperidin-4-ylamino)propanoyl]-N-cyclohexyl-N-{2-[methoxy(methyl)amino]ethyl}piperidin-4-amine hydrochloride is obtained.

Melting point=166° C.; M+H⁺=534.

### EXAMPLE 4

### N-[1-(4-chloro-N-piperidin-4-yl-D-phenylalanyl)piperidin-4-yl]-N-cyclohexyl-2-ethyl-butanamide hydrochloride (compound No. 19)

4.1: tert-butyl 4-[cyclohexyl(2-ethyl-butanoyl)amino]piperidine-1-carboxylate

1.5 g of tert-butyl 4-(cyclohexylamino)-piperidine-1-carboxylate, obtained in step 1.1, are placed in 27 ml of dichloromethane under N₂ at 0° C. 0.89 ml of triethylamine is added, followed by 0.73 ml of 2-ethylbutyric acid chloride. Stirring is maintained at ambient temperature for 16 h. After evaporation to dryness and hydrolysis, extraction is carried out with ethyl acetate until the aqueous phase is completely depleted. The organic phase is washed with a saturated aqueous sodium chloride solution, dried over MgSO₄ and concentrated to dryness. The crude obtained is chromatographed on silica gel, elution being carried out with a gradient of methanol in dichloromethane ranging from 0% to 3%. 1.39 g of tert-butyl 4-[cyclohexyl(2-ethylbutanoyl)amino]piperidine-1-carboxylate are obtained.

4.2: N-cyclohexyl-2-ethyl-N-piperidin-4-ylbutanamide

1.5 g of tert-butyl 4-[cyclohexyl(2-ethyl-butanoyl)amino]piperidine-1-carboxylate are placed in 9.9 ml of 4N hydrochloric acid in dioxane. The reaction medium is stirred at ambient temperature for 16 h. After evaporation to dryness, 1N sodium hydroxide is added up to a pH of 10, and the extraction is carried out with ethyl acetate until the aqueous phase is completely depleted. The organic phase is washed with a saturated sodium chloride solution. After drying over MgSO₄ and evaporation to dryness, the crude is chromatographed on silica gel, elution being carried out with a gradient of methanol in dichloromethane ranging from 0% to 5%. 1.2 g of N-cyclohexyl-2-ethyl-N-piperidin-4-ylbutanamide are obtained.

4.3: tert-butyl 4-[((1R)-1-(4-chlorobenzyl)-2-{4-[cyclohexyl(2-ethylbutanoyl)amino]piperidin-1-yl}-2-oxoethyl)amino]piperidine-1-carboxylate

0.3 g of N-cyclohexyl-2-ethyl-N-piperidin-4-ylbutanamide is dissolved in 9 ml of dichloromethane in the presence of 0.36 g of 4-chloro-N-(1-Boc-piperidin-4-yl)-D-phenylalanine (obtained in step 3.7), of 0.128 g of hydroxybenzotriazole, of 0.182 g of 1-(3-dimethylaminopropyl)-3-ethylcarbodiimide hydrochloride and of 0.49 ml of diisopropylethylamine. The mixture is stirred at ambient temperature for 16 h. After concentration and hydrolysis, extraction is carried out with ethyl acetate until the aqueous phase is completely depleted. The organic phase is washed with water and then with a saturated sodium chloride solution. After drying over MgSO₄ and concentration to dryness, the crude is chromatographed on silica gel, elution being carried out with a gradient of methanol in dichloromethane ranging from 0% to 5%, to give 0.23 g of tert-butyl 4-[((1R)-1-(4-chlorobenzyl)-2-(4-{cyclohexyl(2-ethylbutanoyl)amino]piperidin-1-yl}-2-oxoethyl)amino]piperidine-1-carboxylate.

4.4: N-[1-(4-chloro-N-piperidin-4-yl-D-phenylalanyl)piperidin-4-yl]-N-cyclohexyl-2-ethylbutanamide

0.23 g of tert-butyl 4-[((1R)-1-(4-chlorobenzyl)-2-{4-[cyclohexyl(2-ethylbutanoyl)-amino]piperidin-1-yl}-2-oxoethyl)amino]piperidine-1-carboxylate is placed in 1.35 ml of 4N hydrochloric acid in dioxane. The reaction medium is stirred at ambient temperature for 16 h. After evaporation to dryness, 1N sodium hydroxide is added up to a pH of 10, and extraction is carried out with ethyl acetate until the aqueous phase is completely depleted. The organic phase is washed with a saturated sodium chloride solution. After drying over MgSO₄ and evaporation to dryness, the crude is chromatographed on silica gel, elution being carried out with a gradient of methanol/aqueous ammonia in dichloromethane ranging from 0% to 5/0.5/95. 0.16 g of N-[1-(4-chloro-N-piperidin-4-yl-D-phenylalanyl)piperidin-4-yl]-N-cyclohexyl-2-ethylbutanamide is obtained.

4.5: N-[1-(4-chloro-N-piperidin-4-yl-D-phenylalanyl)piperidin-4-yl]-N-cyclohexyl-2-ethylbutanamide hydrochloride

0.16 g of N-[1-(4-chloro-N-piperidin-4-yl-D-phenylalanyl)piperidin-4-yl]-N-cyclohexyl-2-ethylbutanamide is placed in 2 ml of dichloromethane, and 5.5 ml of 0.1N hydrochloric acid in isopropanol are added. After concentration to dryness, the residue is taken up with ethyl acetate and triturated. The crystals obtained are filter-dried and rinsed with ethyl acetate. After drying over P₂O₅, 0.13 g of N-[1-(4-chloro-N-piperidin-4-yl-D-phenylalanyl)piperidin-4-yl]-N-cyclohexyl-2-ethylbutanamide hydrochloride is obtained.

Melting point=285° C.; M+H⁺=545; [α]_{Hg365}²⁰=+5° (c=0.8945 g/100 ml, DMSO).

### EXAMPLE 5

### N-{1-[4-chloro-N-(tetrahydro-2H-pyran-4-yl)-D-phenylalanyl]piperidin-4-yl}-N-cyclohexyl-N′,N′-diethylurea hydrochloride (compound No. 3)

5.1: N-{1-[4-chloro-N-(tetrahydro-2H-pyran-4-yl)-D-phenylalanyl]piperidin-4-yl}-N-cyclohexyl-N′,N′-diethylurea

0.23 g of N-[1-(4-chloro-D-phenylalanyl)-piperidin-4-yl]-N-cyclohexyl-N′,N′-diethylurea, obtained in step 1.5 above, is dissolved in 3 ml of dichloromethane in the presence of 0.05 ml of tetrahydro-4H-4-one and of 0.22 g of sodium triacetoxyborohydride under N₂. Stirring is maintained at ambient temperature for 18 h. After the addition of 0.044 g of ketone and 0.222 g of triacetoxyborohydride, the reaction medium is stirred for 48 h. After the addition of 2 ml of methanol, the solution is loaded onto a cartridge containing 4 g of DOWEX® 50X2 resin. The resin is washed with THF, with water and then with methanol. The expected compound is then released with 2N aqueous ammonia in methanol. After concentration to dryness, 0.23 g of N-{1-[4-chloro-N-(tetrahydro-2H-pyran-4-yl)-D-phenylalanyl]piperidin-4-yl}-N-cyclohexyl-N′,N′-diethylurea is obtained.

5.2: N-{1-[4-chloro-N-(tetrahydro-2H-pyran-4-yl)-D-phenylalanyl]piperidin-4-yl}-N-cyclohexyl-N′,N′-diethylurea hydrochloride

0.23 g of N-{1-[4-chloro-N-(tetrahydro-2H-pyran-4-yl)-D-phenylalanyl]piperidin-4-yl}-N-cyclohexyl-N′,N′-diethylurea is mixed with 0.37 ml of 2N hydrochloric acid in diethyl ether. The solution is triturated. The crystals obtained are rinsed with diethyl ether and filter-dried. 0.22 g of N-{1-[4-chloro-N-(tetrahydro-2H-pyran-4-yl)-D-phenylalanyl]-piperidin-4-yl}-N-cyclohexyl-N′,N′-diethylurea hydrochloride is obtained in the form of a white solid.

Melting point>200° C.; M+H⁺=547; [α]_{D}²⁰=+2.7° (c=0.537 g/100 ml, DMSO).

### EXAMPLE 6

### N-{1-[N-(4-aminocyclohexyl)-4-chloro-D-phenylalanyl]piperidin-4-yl}-N-cyclohexyl-N′,N′-diethylurea hydrochloride (compound No. 2)

6.1: N-{1-[N-(4-Boc-aminocyclohexyl)-4-chloro-D-phenylalanyl]piperidin-4-yl}-N-cyclohexyl-N′,N′-diethylurea

0.23 g of N-[1-(4-chloro-D-phenylalanyl)-piperidin-4-yl]-N-cyclohexyl-N′,N′-diethylurea, obtained in step 1.5 above, is dissolved in 3 ml of dichloromethane in the presence of 0.12 g of N-4-Boc-aminocyclohexanone and of 0.22 g of sodium triacetoxyborohydride under N₂. Stirring is maintained at ambient temperature for 18 h. After the addition of 2 ml of methanol, the solution is loaded onto a cartridge containing 4 g of DOWEX® 50X2 resin. The resin is washed with THF, with water and then with methanol. The expected compound is then released with 2N aqueous ammonia in methanol. After concentration to dryness, 0.18 g of tert-butyl (4-{[(1R)-1-(4-chloro-benzyl)-2-(4-(cyclohexyl[(diethylamino)carbonyl]amino}-piperidin-1-yl)-2-oxoethyl]amino}cyclohexyl)carbamate is obtained.

6.2: N-{1-[N-(4-aminocyclohexyl)-4-chloro-D-phenylalanyl]piperidin-4-yl}-N-cyclohexyl-N′,N′-diethylurea hydrochloride

0.18 g of tert-butyl (4-{[(1R)-1-(4-chloro-benzyl)-2-(4-{cyclohexyl[(diethylamino)carbonyl]-amino}piperidin-1-yl)-2-oxoethyl]amino}cyclohexyl)-carbamate is placed in 2 ml of diethyl ether, and 0.77 ml of 2N hydrochloric acid in diethyl ether is added. The reaction medium is stirred at ambient temperature for 18 h. The crystals obtained are rinsed with diethyl ether and filter-dried. 0.14 g of a mixture of cis and trans isomers of N-{1-[N-(4-aminocyclohexyl)-4-chloro-D-phenylalanyl]piperidin-4-yl}-N-cyclohexyl-N′,N′-diethylurea hydrochloride is obtained.

Melting point=195° C.; M+H⁺=560.

### EXAMPLE 7

### N-[1-(N-8-azabicyclo[3.2.1]oct-3-yl-4-chloro-D-phenylalanyl)piperidin-4-yl]-N-cyclo-hexyl-N′,N′-diethylurea hydrochloride (compound No. 4)

7.1: tert-butyl 3-{[(1R)-1-(4-chlorobenzyl)-2-(4-{cyclohexyl[(diethylamino)carbonyl]amino}-piperidin-1-yl)-2-oxoethyl]amino}-8-azabicyclo-[3.2.1]octane-8-carboxylate

0.46 g of N-[1-(4-chloro-D-phenylalanyl)-piperidin-4-yl]-N-cyclohexyl-N′,N′-diethylurea, obtained in step 1.5 above, is dissolved in 10 ml of dichloromethane in the presence of 0.034 g of Boc-nortropinone and of 0.42 g of sodium triacetoxy-borohydride under N₂. Stirring is maintained at ambient temperature for 18 h. 0.10 g of Boc-nortropinone and 0.10 g of sodium triacetoxyborohydride are added. Stirring is maintained for 24 h. After hydrolysis, extraction is carried out with dichloromethane until the aqueous phase is completely depleted. The organic phase is washed with H₂O and then with an aqueous sodium chloride solution. After drying over MgSO₄ and concentration to dryness, the crude obtained is chromatographed on silica gel, elution being carried out with a 90/10 mixture of cyclohexane and ethyl acetate. 0.37 g of tert-butyl 3-{[(1R)-1-(4-chloro-benzyl)-2-(4-{cyclohexyl[(diethylamino)carbonyl]amino}piperidin-1-yl)-2-oxoethyl]amino}-8-azabicyclo[3.2.1]-octane-8-carboxylate is obtained.

7.2: N-[1-(N-8-azabicyclo[3.2.1]oct-3-yl-4-chloro-D-phenylalanyl)piperidin-4-yl]-N-cyclohexyl-N′,N′-diethylurea hydrochloride

0.37 g of tert-butyl 3-[([(1R)-1-(4-chloro-benzyl)-2-(4-{cyclohexyl[(diethylamino)carbonyl]amino}-piperidin-1-yl)-2-oxoethyl]amino}-8-azabicyclo[3.2.1]-octane-8-carboxylate is placed in 2 ml of diethyl ether, and 2.74 ml of 2N hydrochloric acid in diethyl ether are added. The reaction medium is stirred at ambient temperature for 18 h. A further 2 ml of 2N hydrochloric acid in diethyl ether are added. The crystals obtained are rinsed with diethyl ether and filter-dried. 0.30 g of N-[1-(N-8-azabicyclo[3.2.1]oct-3-yl-4-chloro-D-phenylalanyl)piperidin-4-yl]-N-cyclohexyl-N′,N′-diethylurea hydrochloride is obtained.

Melting point=182° C.; M+H⁺=572; [α]_{D}²⁰=+9.2° (c=0.667 g/100 ml, DMSO).

### EXAMPLE 8

### N-{1-[4-chloro-N-(1-isobutyl-piperidin-4-yl)-D-phenylalanyl]piperidin-4-yl}-N-cyclohexyl-N′,N′-diethylurea hydrochloride (compound No. 6)

8.1: N-{1-[4-chloro-N-(1-isobutylpiperidin-4-yl)-D-phenylalanyl]piperidin-4-yl}-N-cyclohexyl-N′,N′-diethylurea

0.25 g of N-[1-(4-chloro-N-piperidin-4-yl-D-phenylalanyl)piperidin-4-yl]-N-cyclohexyl-N′,N′-diethylurea, obtained in step 1.7 above, is dissolved in 4 ml of dichloromethane, in the presence of 0.05 ml of isobutyraldehyde and of 0.16 g of sodium triacetoxyborohydride under N₂. Stirring is maintained at ambient temperature for 3 days. After hydrolysis with an aqueous sodium hydroxide solution up to a pH of 10, extraction is carried out with ethyl acetate until the aqueous phase is completely depleted. The organic phase is washed with H₂O and then with an aqueous sodium chloride solution. After drying over MgSO₄ and concentration to dryness, the crude obtained is chromatographed on silica gel, elution being carried out with a gradient of a 9/1/0.1 mixture of dichloromethane, methanol and aqueous ammonia in dichloromethane ranging from 0% to 100%. 0.14 g of N-{1-[4-chloro-N-(1-isobutylpiperidin-4-yl)-D-phenylalanyl]piperidin-4-yl}-N-cyclohexyl-N′,N′-diethylurea is obtained.

8.2: N-{1-[4-chloro-N-(1-isobutylpiperidin-4-yl)-D-phenylalanyl] piperidin-4-yl}-N-cyclohexyl-N′,N′-diethylurea hydrochloride

0.14 g of is placed in 2 ml of dichloromethane, and 4.48 ml of 0.1N hydrochloric acid in isopropanol are added. After concentration to dryness, the residue is triturated in a mixture of diethyl ether and ethyl acetate. The crystals obtained are rinsed with diethyl ether, filter-dried, and dried over P₂O₅. 0.115 g of N-(1-[4-chloro-N-(1-isobutyl-piperidin-4-yl)-D-phenylalanyl]piperidin-4-yl}-N-cyclohexyl-N′,N′-diethylurea hydrochloride is obtained.

Melting point >250° C.; M+H⁺=602; [α]_{D}²⁰=+10.6° (c=0.881 g/100 ml, DMSO).

### EXAMPLE 9

### 1-[(2R)-3-(4-chlorophenyl)-1-methylene-2-(piperidin-4-ylamino)propyl]-N-cyclohexyl-N-(4-methoxyphenyl)piperidin-4-amine hydrochloride (compound No. 40)

9.1: tert-butyl 4-[(4-methoxyphenyl)amino]-piperidine-1-carboxylate

2.0 g of 1-Boc-piperidone are placed in 85 ml of acetic acid under N₂ in the presence of 6.47 g of 4-methoxyaniline, of 23 g of sodium sulphate and of 10.3 g of sodium triacetoxyborohydride, and the reaction medium is stirred at ambient temperature for 16 h. After concentration to dryness, 30% aqueous sodium hydroxide is added up to a basic pH. Extraction is carried out with ethyl acetate until the aqueous phase is completely depleted. The organic phase is washed with water and then with a saturated sodium chloride solution. After drying over MgSO₄ and concentration to dryness, 9 g of tert-butyl 4-[(4-methoxyphenyl)amino]piperidine-1-carboxylate are obtained, which product is subsequently used as it is.

9.2: tert-butyl 4-[cyclohexyl(4-methoxy-phenyl)amino]piperidine-1-carboxylate

5.0 g of tert-butyl 4-[(4-methoxyphenyl)-amino]piperidine-1-carboxylate are placed in 55 ml of dichloromethane under N₂ in the presence of 5.78 ml of cyclohexanone and of 4.84 g of sodium triacetoxy-borohydride. After stirring for 18 h, 2.9 ml of cyclohexanone and 2.4 g of sodium triacetoxyborohydride are added, and the reaction medium is stirred at ambient temperature for 5 days. After the addition of 20 ml of methanol and of approximately 0.5 g of citric acid and 50 ml of water, and stirring for 18 h, extraction is carried out with dichloromethane until the aqueous phase is completely depleted. After drying over Na₂SO₄ and concentration to dryness, the crude obtained is chromatographed on silica gel, elution being carried out with a 7/3 mixture of heptane/EtOAc. 4 g of tert-butyl 4-[cyclohexyl(4-methoxyphenyl)amino]-piperidine-1-carboxylate are obtained as a mixture.

9.3: N-cyclohexyl-N-(4-methoxyphenyl)-piperidin-4-amine

10 g of tert-butyl 4-[cyclohexyl(4-methoxy-phenyl)amino]piperidine-1-carboxylate are placed in 50 ml of 4N hydrochloric acid in dioxane. The reaction medium is stirred at ambient temperature for 18 h. After evaporation to dryness, the residue is chromatographed, elution being carried with a mixture of dichloromethane, methanol and aqueous ammonia ranging from 95/5/0.5 to 85/15/1.5. 2.1 g of N-cyclohexyl-N-(4-methoxyphenyl)piperidin-4-amine are obtained.

9.4: tert-butyl 4-[((1R)-1-(4-chlorobenzyl)-2-{4-[cyclohexyl(4-methoxyphenyl)amino]piperidin-1-yl}-2-oxoethyl)amino]piperidine-1-carboxylate

0.29 g of N-cyclohexyl-N-(4-methoxyphenyl)-piperidin-4-amine, obtained in step 9.3, is dissolved in 10 ml of dichloromethane in the presence of 0.38 g of N-[1-(tert-butoxycarbonyl)piperidin-4-yl]-4-chloro-D-phenylalanine, obtained in step 3.7, of 0.14 g of hydroxybenzotriazole, of 0.19 g of 1-(3-dimethylamino-propyl)-3-ethylcarbodiimide hydrochloride and of 0.17 ml of diisopropylethylamine. The mixture is stirred at ambient temperature for 16 h. After hydrolysis, extraction is carried out with dichloromethane until the aqueous phase is completely depleted. The organic phase is washed with a saturated aqueous sodium hydrogen carbonate solution. After drying over Na₂SO₄ and concentrated to dryness, the crude obtained is chromatographed, elution being carried out with a mixture of dichloromethane, methanol and aqueous ammonia ranging from 95/5/0 to 9/1/0.5. 0.32 g of tert-butyl 4-[((1R)-1-(4-chlorobenzyl)-2-{4-[cyclohexyl(4-methoxyphenyl)amino]piperidin-1-yl]-2-oxoethyl)amino]piperidine-1-carboxylate is obtained.

9.5: 1-[(2R)-3-(4-chlorophenyl)-2-(piperidin-4-ylamino)propanoyl]-N-cyclohexyl-N-(4-methoxyphenyl)-piperidin-4-amine

0.32 g of tert-butyl 4-[((1R)-1-(4-chloro-benzyl)-2-{4-[cyclohexyl(4-methoxyphenyl)amino]-piperidin-1-yl}-2-oxoethyl)amino]piperidine-1-carboxylate is placed in 5 ml of dioxane, and 1.22 ml of 4N hydrochloric acid in dioxane are added. The reaction medium is stirred at ambient temperature for 18 h. After evaporation to dryness, the residue is taken up with methanol and again concentrated to dryness. The crude obtained is chromatographed, elution being carried out with a gradient of a mixture of methanol and aqueous ammonia in dichloromethane ranging from 95/5/0.5 to 9/1/0.1. 0.176 g of 1-[(2R)-3-(4-chlorophenyl)-2-(piperidin-4-ylamino)propanoyl]-N-cyclohexyl-N-(4-methoxyphenyl)piperidin-4-amine is obtained.

9.6: 1-[(2R)-3-(4-chlorophenyl)-2-(piperidin-4-ylamino)propanoyl]-N-cyclohexyl-N-(4-methoxyphenyl)-piperidin-4-amine hydrochloride

0.17 g of 1-[(2R)-3-(4-chlorophenyl)-2-(piperidin-4-ylamino)propanoyl]-N-cyclohexyl-N-(4-methoxyphenyl)piperidin-4-amine is placed in 5 ml of dichloromethane, and 3.2 ml of 0.1N hydrochloric acid in isopropanol are added. After concentration to dryness, the residue is recrystallized from ethanol. 0.036 g of 1-[(2R)-3-(4-chlorophenyl)-2-(piperidin-4-ylamino)propanoyl]-N-cyclohexyl-N-(4-methoxyphenyl)-piperidin-4-amine hydrochloride is obtained. Melting point=195° C.; M+H⁺=553.

### EXAMPLE 10

### 1-[(2R)-3-(4-chlorophenyl)-2-(piperidin-4-ylamino)propanoyl]-N-cyclohexyl-N-[2-(1H-imidazol-1-yl)ethyl]piperidin-4-amine hydrochloride (compound No. 44)

10.1: tert-butyl 4-[cyclohexyl(2-ethoxy-2-oxoethyl)amino]piperidine-1-carboxylate

4.64 g of tert-butyl 4-(cyclohexylamino)-piperidine-1-carboxylate, obtained in step 1.1, are dissolved in 164 ml of dichloromethane, and 9.77 ml of ethyl oxoacetate are added. 13.93 g of sodium triacetoxyborohydride are slowly added. Stirring is maintained at ambient temperature for 18 h. A further 3.25 ml of glyoxylic acid ethyl ester and 3.48 g of sodium triacetoxyborohydride are added. After stirring for 72 h, the reaction medium is treated with methanol and concentrated to dryness. The residue is taken up with a saturated aqueous sodium hydrogen carbonate solution. Extraction is carried out with ethyl acetate until the aqueous phase is completely depleted. The organic phase is washed with a saturated aqueous sodium chloride solution. After drying over MgSO₄ and concentration to dryness, the crude obtained is chromatographed on silica gel, elution being carried out with a gradient of methanol in dichloromethane ranging from 0% to 10%. 6.44 g of tert-butyl 4-[cyclohexyl(2-ethoxy-2-oxoethyl)amino]piperidine-1-carboxylate are obtained.

10.2: tert-butyl 4-[cyclohexyl(2-hydroxyethyl)amino]piperidine-1-carboxylate

6.44 g of tert-butyl 4-[cyclohexyl(2-ethoxy-2-oxoethyl)amino]piperidine-1-carboxylate are placed in 175 ml of diethyl ether at 0° C. under N₂. 29.71 ml of 1N lithium aluminium hydride in diethyl ether are added slowly. After stirring at 0° C. for 1 h, a saturated aqueous potassium sulphate solution is added up to a pH of 5-6. After the addition of 1N aqueous sodium hydroxide, extraction is carried out with dichloromethane until the aqueous phase is completely depleted. The organic phase is washed with water and then with a saturated aqueous sodium chloride solution. After drying over MgSO₄ and concentration to dryness, 4.04 g of tert-butyl 4-[cyclohexyl(2-hydroxyethyl)-amino]piperidine-1-carboxylate are added, which product is used as it is in the subsequent synthesis.

10.3: tert-butyl 4-(cyclohexyl(2-[(methylsulphonyl)oxy]ethyl}amino)piperidine-1-carboxylate

0.75 g of tert-butyl 4-[cyclohexyl(2-hydroxyethyl)amino]piperidine-1-carboxylate is dissolved in 23 ml of diethyl ether. 0.63 ml of triethylamine and 0.28 ml of mesyl chloride are added. After stirring at ambient temperature for 2 h, the triethylamine hydrochloride formed is filtered off and the filtrate is concentrated to dryness. 0.82 g of tert-butyl 4-(cyclohexyl{2-[(methylsulphonyl)oxy]-ethyl}amino)piperidine-1-carboxylate is obtained, which product is used as it is in the subsequent synthesis.

10.4: tert-butyl 4-{cyclohexyl[2-(1H-imidazol-1-yl)ethyl]amino}piperidine-1-carboxylate

0.82 g of tert-butyl 4-(cyclohexyl{2-[(methylsulphonyl)oxy]ethyl}amino)piperidine-1-carboxylate is dissolved in 4 ml of a mixture of acetonitrile/dimethylformamide (1/1), and then 0.41 g of sodium 1,2,4-triazole is added. After stirring at ambient temperature for 18 h, hydrolysis is performed and extraction is carried out with dichloromethane until the aqueous phase is completely depleted. The organic phase is washed with water. After drying over MgSO₄ and concentration to dryness, the crude obtained is chromatographed on silica gel, elution being carried out with a gradient of methanol in dichloromethane from 0% to 10%. 0.37 g of tert-butyl 4-(cyclohexyl[2-(1H-imidazol-1-yl)ethyl]amino}piperidine-1-carboxylate is obtained in the form of red crystals.

10.5: N-cyclohexyl-N-[2-(1H-imidazol-1-yl)ethyl]piperidin-4-amine

0.45 g of tert-butyl 4-{cyclohexyl[2-(1H-imidazol-1-yl)ethyl]amino}piperidine-1-carboxylate is placed in 12 ml of 4N hydrochloric acid in dioxane. The reaction medium is stirred at ambient temperature for 18 h. After evaporation to dryness, the residue is taken up with methanol and again concentrated to dryness. This operation is repeated several times. 0.51 g of N-cyclohexyl-N-[2-(1H-imidazol-1-yl)ethyl]piperidin-4-amine is obtained, which product is subsequently used as it is.

10.6: tert-butyl 4-([(1R)-1-(4-chlorobenzyl)-2-(4-{cyclohexyl[2-(1H-imidazol-1-yl)ethyl]amino}-piperidin-1-yl)-2-oxoethyl]amino}piperidine-1-carboxylate

0.05 g of N-cyclohexyl-N-[2-(1H-imidazol-1-yl)ethyl]piperidin-4-amine, obtained in step 10.5, is dissolved in 13 ml of dichloromethane in the presence of 0.51 g of N-[1-(tert-butoxycarbonyl)piperidin-4-yl]-4-chloro-D-phenylalanine, obtained in step 3.7, of 0.18 g of hydroxybenzotriazole, of 0.25 g of 1-(3-dimethylaminopropyl)-3-ethylcarbodiimide hydrochloride and of 0.92 ml of diisopropylethylamine. The mixture is stirred at ambient temperature for 16 h. After hydrolysis, extraction is carried out with dichloromethane until the aqueous phase is completely depleted. The organic phase is washed with H₂O and then a saturated aqueous sodium chloride solution. After drying over MgSO₄ and concentration to dryness, the crude obtained is chromatographed, elution being carried out with a gradient of methanol in dichloromethane ranging from 0% to 10%. 0.37 g of tert-butyl 4-{[(1R)-1-(4-chlorobenzyl)-2-(4-(cyclohexyl[2-(1H-imidazol-1-yl)ethyl]amino}piperidin-1-yl)-2-oxoethyl]amino)piperidine-1-carboxylate is obtained.

10.7: 1-[(2R)-3-(4-chlorophenyl)-2-(piperidin-4-ylamino)propanoyl]-N-cyclohexyl-N-[2-(1H-imidazol-1-yl)ethyl]piperidin-4-amine

0.37 g of tert-butyl 4-{[(1R)-1-(4-chlorobenzyl)-2-(4-{cyclohexyl[2-(1H-imidazol-1-yl)ethyl]amino}piperidin-1-yl)-2-oxoethyl]amino}-piperidine-1-carboxylate is placed in 5.7 ml of dioxane, and 1.43 ml of 4N hydrochloric acid in dioxane are added. The reaction medium is stirred at ambient temperature for 18 h. After evaporation to dryness, the residue is taken up with a saturated aqueous sodium hydrogen carbonate solution. Extraction is carried out with dichloromethane until the aqueous phase is completely depleted. The organic phase is washed with H₂O. After drying over MgSO₄ and concentration to dryness, the crude obtained is chromatographed, elution being carried out with a gradient of a mixture of methanol and aqueous ammonia in dichloromethane ranging from 100/0/0 to 8/2/0.2. 0.19 g of 1-[(2R)-3-(4-chlorophenyl)-2-(piperidin-4-ylamino)propanoyl]-N-cyclohexyl-N-[2-(1H-imidazol-1-yl)ethyl]piperidin-4-amine is obtained.

10.8: 1-[(2R)-3-(4-chlorophenyl)-2-(piperidin-4-ylamino)propanoyl]-N-cyclohexyl-N-[2-(1H-imidazol-1-yl)ethyl]piperidin-4-amine hydrochloride

0.19 g of 1-[(2R)-3-(4-chlorophenyl)-2-(piperidin-4-ylamino)propanoyl]-N-cyclohexyl-N-[2-(1H-imidazol-1-yl)ethyl]piperidin-4-amine is placed in 5 ml of methanol, and 3.5 ml of 0.1N hydrochloric acid in isopropanol are added. After evaporation to dryness, the reaction medium is triturated in diethyl ether, the precipitate obtained is then filter-dried, and rinsed with diethyl ether. The hydrochloride thus obtained is dried over P₂O₅ under reduced pressure. 0.175 g of 1-[(2R)-3-(4-chlorophenyl)-2-(piperidin-4-ylamino)-propanoyl]-N-cyclohexyl-N-[2-(1H-imidazol-1-yl)ethyl]piperidin-4-amine hydrochloride is obtained in the form of a white solid.

Melting point=162° C.; M+H⁺=541; [α]_{D}²⁰=−3.9° (c=0.418 g/100 ml, DMSO).

### EXAMPLE 11

### N-{1-[N-(cis-4-aminocyclohexyl)-4-chloro-D-phenylalanyl]piperidin-4-yl}-N-cyclohexyl-2,2-dimethylhydrazinecarboxamide hydrochloride (compound No. 71)

11.1: tert-butyl 4-{cyclohexyl[(2,2-dimethylhydrazino)carbonyl]amino}piperidine-1-carboxylate

0.43 ml of diphosgene is placed in 18 ml of dichloromethane at 0° C. under N₂. A solution of 1.0 g of tert-butyl 4-(cyclohexylamino)piperidine-1-carboxylate and of 2.47 ml of triethylamine is added dropwise. The solution is stirred at ambient temperature for 2 h. The reaction medium is again placed at 0° C. and 0.43 ml of diphosgene is again added. After stirring at ambient temperature for 2 h, 5.39 ml of dimethylhydrazine are added. The mixture is stirred at ambient temperature for 18 h. 30 ml of 0.5N hydrochloric acid are added. Extraction is carried out with dichloromethane until the aqueous phase is completely depleted. After drying over MgSO₄ and concentration to dryness, the residue obtained is chromatographed on silica gel, elution being carried out with a gradient of methanol in dichloromethane ranging from 2% to 10%, to give 0.28 g of tert-butyl 4-{cyclohexyl[(2,2-dimethylhydrazino)-carbonyl]amino}piperidine-1-carboxylate.

11.2: N-cyclohexyl-2,2-dimethyl-N-piperidin-4-ylhydrazinecarboxamide

0.28 g of tert-butyl 4-{cyclohexyl[(2,2-dimethylhydrazino)carbonyl]amino}piperidine-1-carboxylate is placed in 3.8 ml of 4N hydrochloric acid in dioxane. The reaction medium is stirred at ambient temperature for 18 h. After evaporation to dryness, the residue is taken up with a 1N sodium hydroxide solution and extraction is carried out with dichloromethane until the aqueous phase is completely depleted. After drying over MgSO₄, 0.2 g of N-cyclohexyl-2,2-dimethyl-N-piperidin-4-ylhydrazinecarboxamide is obtained, which product is subsequently used as it is.

11.3: Methyl N-{cis-4-[(tert-butoxycarbonyl)-amino]cyclohexyl}-4-chloro-D-phenylalaninate

10 g of H-p-chloro-D-Cl-Phe-OMe,HCl, and 8.5 g of tert-butyl (4-oxocyclohexyl)carbamate are placed in 200 ml of dichloromethane. 11 g of NaBH(OAc)₃ and 5.57 ml of NEt₃ are added. Stirring is maintained at ambient temperature for 18 h. The solution is hydrolysed with a 1N aqueous sodium hydroxide solution and extracted with dichloromethane until the aqueous phase is completely depleted. After drying over MgSO₄ and concentration to dryness, the crude obtained is chromatographed on silica gel, elution being carried out with a gradient of a mixture of EtOAc/MeOH in CH₂Cl₂ ranging from 95/5/1 to 85/15/3 (CH₂Cl₂/EtOAc/MeOH). 5.7 g of methyl N-{cis-4-[(tert-butoxycarbonyl)amino]-cyclohexyl}-4-chloro-D-phenylalaninate are obtained.

11.4: N-(cis-4-[(tert-butoxycarbonyl)amino]-cyclohexyl}-4-chloro-D-phenylalanine

5.5 g of methyl N-{cis-4-[(tert-butoxy-carbonyl)amino]cyclohexyl}-4-chloro-D-phenylalaninate are placed in 133 ml of MeOH, and then 40.15 ml of 1N aqueous sodium hydroxide are added. Stirring is maintained at ambient temperature for 18 h. After evaporation of the MeOH, 4 eq of a 1N aqueous hydrochloric acid solution are added. The white precipitate thus obtained is filtered off under cold conditions and rinsed with cold water. After drying over P₂O₅, 3.8 g of N-{cis-4-[(tert-butoxycarbonyl)-amino]cyclohexyl}-4-chloro-D-phenylalanine are obtained.

11.5: tert-butyl (cis-4-{[(1R)-1-(4-chlorobenzyl)-2-(4-(cyclohexyl[(2,2-dimethyl-hydrazino)carbonyl]amino}piperidin-1-yl)-2-oxoethyl]amino}cyclohexyl)carbamate

0.22 g of N-cyclohexyl-2,2-dimethyl-N-piperidin-4-ylhydrazinecarboxamide, obtained in step 11.2, is dissolved in 10 ml of dichloromethane in the presence of 0.28 g of N-{cis-4-[(tert-butoxycarbonyl)-amino]cyclohexyl}-4-chloro-D-phenylalanine, obtained in step 11.4, of 0.11 g of hydroxybenzotriazole, of 0.24 g of 1-(3-dimethylaminopropyl)-3-ethylcarbodiimide hydrochloride and of 0.5 ml of diisopropylethylamine and 0.82 ml of hydrochloric acid in dioxane. The mixture is stirred at ambient temperature for 18 h. After hydrolysis with an aqueous sodium hydrogen carbonate solution, extraction is carried out with ethyl acetate until the aqueous phase is completely depleted. After drying over MgSO₄ and concentration to dryness, the crude obtained is chromatographed, elution being carried out with a gradient of methanol in dichloromethane ranging from 1% to 4%. 0.22 g of tert-butyl (cis-4-([(1R)-1-(4-chlorobenzyl)-2-(4-{cyclohexyl[(2,2-dimethylhydrazino)carbonyl]amino}-piperidin-1-yl)-2-oxoethyl]amino]cyclohexyl)carbamate is obtained.

11.6: N-{1-[N-(cis-4-aminocyclohexyl)-4-chloro-D-phenylalanyl]piperidin-4-yl}-N-cyclohexyl-2,2-dimethylhydrazinecarboxamide

0.22 g of tert-butyl (cis-4-{[(1R)-1-(4-chlorobenzyl)-2-(4-{cyclohexyl[(2,2-dimethylhydrazino)-carbonyl]amino}piperidin-1-yl)-2-oxoethyl]amino}-cyclohexyl)carbamate is placed in 1.7 ml of dioxane, and 1.43 ml of 4N hydrochloric acid in dioxane are added. The reaction medium is stirred at ambient temperature for 3 h. After evaporation to dryness, the residue is taken up with a 1N aqueous sodium hydroxide solution. Extraction is carried out with dichloromethane until the aqueous phase is completely depleted. After drying over MgSO₄ and concentration to dryness, the crude obtained is chromatographed, elution being carried out with a gradient of a mixture of methanol and aqueous ammonia in dichloromethane ranging from 95/5/0.5 to 9/1/0.1. 0.05 g of N-{1-[N-(cis-4-aminocyclohexyl)-4-chloro-D-phenylalanyl]piperidin-4-yl}-N-cyclohexyl-2,2-dimethylhydrazinecarboxamide is obtained.

11.7: N-{1-[N-(cis-4-aminocyclohexyl)-4-chloro-D-phenylalanyl]piperidin-4-yl}-N-cyclohexyl-2,2-dimethylhydrazinecarboxamide hydrochloride

0.05 g of N-1-[1N-(cis-4-aminocyclohexyl)-4-chloro-D-phenylalanyl]piperidin-4-yl}-N-cyclohexyl-2,2-dimethylhydrazinecarboxamide is placed in 10 ml of diethyl ether, and 0.09 ml of 2N hydrochloric acid in diethyl ether is added. The precipitate obtained is and dried over P₂O₅. 0.06 g of N-{1-[N-(cis-4-amino-cyclohexyl)-4-chloro-D-phenylalanyl]piperidin-4-yl}-N-cyclohexyl-2,2-dimethylhydrazinecarboxamide hydrochloride is obtained. Melting point 124° C.; M+H⁺=547.

### EXAMPLE 12

### N-{1-[N-(cis-4-aminocyclohexyl)-4-chloro-D-phenylalanyl]piperidin-4-yl}-N-cycloheptyl-N′,N′-diethylurea hydrochloride (compound No. 33)

12.1: tert-butyl 4-(cycloheptylamino)-piperidine-1-carboxylate

6.98 g of 1-Boc-piperidone are placed in 175 ml of dichloromethane under N₂ in the presence of 4.46 ml of cycloheptylamine and of 9.65 g of sodium triacetoxyborohydride, and the reaction medium is stirred at ambient temperature for 16 h. After the addition of 80 ml of a 0.5N aqueous sodium hydroxide solution, extraction is carried out with ethyl acetate until the aqueous phase is completely depleted. After drying over MgSO₄ and concentration to dryness, 5.6 g of tert-butyl 4-(cycloheptylamino)piperidine-1-carboxylate are obtained, which product is used as it is in the subsequent synthesis.

12.2: tert-butyl 4-{cycloheptyl-[(diethylamino)carbonyl]amino}piperidine-1-carboxylate 0.98 ml of diphosgene is placed in 20 ml of dichloromethane at 0° C. under N₂. A solution of 1.2 g of tert-butyl 4-(cycloheptylamino)piperidine-1-carboxylate and of 5.64 ml of triethylamine is added dropwise. This solution is stirred at 0° C. for 30 min and then at ambient temperature for 3 h. 4.23 ml of diethylamine are then added. The mixture is stirred at ambient temperature for 16 h. After evaporation of the dichloromethane, 50 ml of 0.5N hydrochloric acid are added. Extraction with dichloromethane is carried out until the aqueous phase is completely depleted. After drying over MgSO₄ and concentration to dryness, the residue obtained is chromatographed on silica gel, elution being carried out with a 99/1 and then 98/2 mixture of dichloromethane and methanol, to give 4.18 g of tert-butyl 4-{cycloheptyl[(diethylamino)carbonyl]-amino piperidine-1-carboxylate.

12.3: N-cycloheptyl-N′,N′-diethyl-N-piperidin-4-ylurea

1.6 g of tert-butyl 4-{cycloheptyl-[(diethylamino)carbonyl]amino}piperidine-1-carboxylate is placed in 20.25 ml of 4N hydrochloric acid in dioxane. The reaction medium is stirred at ambient temperature for 16 h. After evaporation to dryness, approximately 10 ml of dichloromethane, 10 ml of tetrahydrofuran, 5 ml of water and 5 ml of methanol are added. 25 g of DOWEX® 50X2 resin are then added. The mixture is left to stir at ambient temperature for 1 h. After washing the resin successively with tetrahydrofuran, dichloromethane and methanol, the expected compound is released with a 2N solution of aqueous ammonia in methanol. After concentration to dryness, 1 g of N-cycloheptyl-N′,N′-diethyl-N-piperidin-4-ylurea is obtained in the form of a red oil.

12.4: tert-butyl (cis-4-{[(1R)-1-(4-chlorobenzyl)-2-(4-{cycloheptyl[(diethylamino)-carbonyl]amino}piperidin-1-yl)-2-oxoethyl]amino}-cyclohexyl)carbamate

0.24 g of N-cycloheptyl-N′,N′-diethyl-N-piperidin-4-ylurea, obtained in step 12.3, is dissolved in 10 ml of dichloromethane in the presence of 0.28 g of N-{cis-4-[(tert-butoxycarbonyl)amino]cyclohexyl}-4-chloro-D-phenylalanine, obtained in step 11.4, of 0.11 g of hydroxybenzotriazole, of 0.23 g of 1-(3-dimethylaminopropyl)-3-ethylcarbodiimide hydrochloride and of 0.5 ml of diisopropylethylamine, and then 0.81 ml of 2N hydrochloric acid in dioxane. The mixture is stirred at ambient temperature for 18 h. After hydrolysis with a saturated aqueous sodium hydrogen carbonate solution, extraction is carried out with ethyl acetate until the aqueous phase is completely depleted. After drying over MgSO₄ and concentration to dryness, the crude obtained is chromatographed, elution being carried out with a gradient of methanol in dichloromethane ranging from 1% to 4%. 0.34 g of tert-butyl (cis-4-{[(1R)-1-(4-chlorobenzyl)-2-(4-{cycloheptyl[(diethylamino)carbonyl]amino}piperidin-1-yl)-2-oxoethyl]amino}cyclohexyl)carbamate is obtained.

12.5: N-{1-[N-(cis-4-aminocyclohexyl)-4-chloro-D-phenylalanyl]piperidin-4-yl}-N-cycloheptyl-N′,N′-diethylurea

0.34 g of tert-butyl (cis-4-{[(1R)-1-(4-chlorobenzyl)-2-(4-{cycloheptyl[(diethylamino)-carbonyl]amino}piperidin-1-yl)-2-oxoethyl]amino}-cyclohexyl)carbamate is placed in 1.43 ml of 4N hydrochloric acid in dioxane. The reaction medium is stirred at ambient temperature for 3 h. After evaporation to dryness, the residue is taken up with a 1N aqueous sodium hydroxide solution. Extraction is carried out with dichloromethane until the aqueous phase is completely depleted. After drying over MgSO₄ and concentration to dryness, the crude obtained is chromatographed, elution being carried out with a gradient of a mixture of methanol and aqueous ammonia in dichloromethane ranging from 95/5/0.5 to 8/2/0.2. 0.22 g of N-(1-[N-(cis-4-aminocyclohexyl)-4-chloro-D-phenylalanyl]piperidin-4-yl}-N-cycloheptyl-N′,N′-diethylurea is obtained.

12.7: N-{1-[N-(cis-4-aminocyclohexyl)-4-chloro-D-phenylalanyl]piperidin-4-yl}-N-cycloheptyl-N′,N′-diethylurea hydrochloride

0.22 g of N-{1-[N-(cis-4-aminocyclohexyl)-4-chloro-D-phenylalanyl]piperidin-4-yl}-N-cycloheptyl-N′,N′-diethylurea is placed in 10 ml of diethyl ether, and 0.38 ml of 2N hydrochloric acid in diethyl ether is added. The precipitate obtained is dried over P₂O₅. 0.23 g of N-{1-[N-(cis-4-aminocyclohexyl)-4-chloro-D-phenylalanyl]piperidin-4-yl}-N-cycloheptyl-N′,N′-diethylurea hydrochloride is obtained.

Melting point=105° C.; M+H⁺=574; [α]_{D}²⁰=+3° (c=0.899 g/100 ml, DMSO).

### EXAMPLE 13

### N-(cis-4-{[(1R)-1-(4-chlorobenzyl)-2-(trans-4-{cyclohexyl[(dimethylamino)-carbonyl]amino}-3-methylpiperidin-1-yl)-2-oxoethyl]-amino}cyclohexyl)acetamide hydrochloride (compound No. 105)

13.1: N-(4-oxocyclohexyl)acetamide

1.5 g of 4-aminocyclohexanone are placed in 50 ml of acetonitrile, and 0.86 ml of acetyl chloride is added, followed by 4.2 g of potassium carbonate. The reaction medium is stirred at ambient temperature for 18 h. After concentration to dryness, the residue is taken up with a 1N aqueous hydrochloric acid solution. Extraction is carried out with dichloromethane until the aqueous phase is completely depleted. The organic phase is washed with a 1N aqueous hydrochloric acid solution. After drying over MgSO₄ and concentration to dryness, 0.45 g of N-(4-oxocyclohexyl)acetamide is obtained, which product is subsequently used as it is.

13.2: 3-methylpiperidin-4-one

12.2 g of 1-benzyl-3-methylpiperidin-4-one are placed in a Parr flask in the presence of 2.44 g of palladium hydroxide in 240 ml of ethanol. The reaction mixture is placed under hydrogen at a pressure of 50 psi and stirred at ambient temperature for 4 h. The solution is filtered over celite and then concentrated to dryness. 6.8 g of 3-methylpiperidin-4-one are thus obtained, which product is subsequently used as it is.

13.3: tert-butyl 3-methyl-4-oxopiperidine-1-carboxylate

6.8 g of 3-methylpiperidin-4-one, 16.7 ml of triethylamine, 19.6 g of di-t-butyl dicarbonate and 0.7 g of dimethylaminopyridine are placed in a mixture of 300 ml of THF and 30 ml of water. Stirring is maintained at ambient temperature for 18 h. After evaporation of the THF, the reaction medium is treated with a saturated aqueous potassium hydrogen sulphate solution to a pH of 1, and then extraction is carried out with ethyl acetate until the aqueous phase is completely depleted. The organic phase is then washed with a saturated aqueous potassium hydrogen sulphate solution, and then with a saturated aqueous sodium hydrogen carbonate solution and, finally, with a saturated aqueous sodium chloride solution. After drying over MgSO₄ and concentration to dryness, the crude obtained is chromatographed on silica gel, elution being carried out with a 98/2 dichloromethane/methanol mixture. 10.3 g of tert-butyl 3-methyl-4-oxopiperidine-1-carboxylate are obtained.

13.4: tert-butyl trans-4-(cyclohexylamino)-3-methylpiperidine-1-carboxylate

7.7 g of tert-butyl 3-methyl-4-oxopiperidine-1-carboxylate and 4.1 ml of cyclohexylamine are placed in 180 ml of methanol, and the pH is adjusted to 6 with 4 ml of acetic acid. 4.5 g of sodium cyanoborohydride are then added. The reaction medium is placed at the reflux of methanol for 18 h. The solution is then hydrolysed with a 1N aqueous sodium hydroxide solution and extracted with ethyl acetate until the aqueous phase is completely depleted. After drying over MgSO₄ and concentration to dryness, the crude obtained is chromatographed on silica gel, with a mixture of dichloromethane/ethyl acetate/methanol/aqueous ammonia ranging from 97/3/0.5/0.05 to 90/10/2/0.2. 1.9 g of tert-butyl trans-4-(cyclohexylamino)-3-methylpiperidine-1-carboxylate and 2.25 g of tert-butyl cis-4-(cyclohexylamino)-3-methylpiperidine-1-carboxylate are obtained.

13.5: tert-butyl trans-4-{cyclohexyl-[(dimethylamino)carbonyl]amino}-3-methylpiperidine-1-carboxylate

0.5 g of tert-butyl trans-4-(cyclohexylamino)-3-methylpiperidine-1-carboxylate is placed in 8.5 ml of dichloromethane, and then 0.35 ml of triethylamine is added and the medium is cooled to 0° C. 0.2 ml of diphosgene is then added slowly. The reaction medium is stirred at 0° C. for 15 min and then at ambient temperature for 5 h. After hydrolysis on a mixture of ice and a 1N aqueous sodium hydroxide solution, extraction is carried out with ethyl acetate until the aqueous phase is completely depleted. The organic phase is washed with H₂O and then with a saturated aqueous sodium chloride solution, dried over MgSO₄, and concentrated to dryness. The crude obtained is dissolved in 8 ml of acetonitrile. 0.71 g of dimethylamine hydrochloride and 1.21 g of potassium carbonate are added. Stirring is maintained at ambient temperature for 40 h. Hydrolysis is performed and extraction is carried out with ethyl acetate until the aqueous phase is completely depleted. The organic phase is washed with water and then with a 1N aqueous hydrochloric acid solution and, finally, with a saturated aqueous sodium chloride solution. It is dried over MgSO₄ and concentrated to dryness. 0.6 g of tert-butyl trans-4-{cyclohexyl[(dimethylamino)carbonyl]-amino}-3-methylpiperidine-1-carboxylate is obtained.

13.6: N-cyclohexyl-N′,N′-dimethyl-N-[trans-3-methylpiperidin-4-yl]urea

0.6 g of tert-butyl trans-4-{cyclohexyl-[(dimethylamino)carbonyl]amino}-3-methylpiperidine-1-carboxylate is placed in 2 ml of dioxane, and then 6.12 ml of 4N hydrochloric acid in dioxane are added and the mixture is left to stir at ambient temperature for 4 h. After concentration to dryness, the residue is taken up with a 1N aqueous sodium hydroxide solution, and extraction is carried out with ethyl acetate until the aqueous phase is completely depleted. The organic phase is washed with a 1N aqueous sodium hydroxide solution, and then with H₂O and, finally, with a saturated aqueous sodium chloride solution. After drying over MgSO₄, the crude obtained is chromatographed on silica gel, with a mixture of dichloromethane/methanol/aqueous ammonia ranging from 10/0/0 to 9/1/0.1. 0.35 g of N-cyclohexyl-N′,N′-dimethyl-N-[trans-3-methylpiperidin-4-yl]urea is obtained.

13.7: tert-butyl [(1R)-1-(4-chlorobenzyl)-2-(trans-4-{cyclohexyl[(dimethylamino)carbonyl]amino}-3-methylpiperidin-1-yl)-2-oxoethyl]carbamate

3.16 g of N-cyclohexyl-N′,N′-dimethyl-N-[trans-3-methylpiperidin-4-yl]urea, obtained in step 13.6, are dissolved in 118 ml of dichloromethane in the presence of 3.5 g of 4-chloro-D-Boc-phenylalanine, of 1.60 g of hydroxybenzotriazole, of 2.27 g of 1-(3-dimethylaminopropyl)-3-ethylcarbodiimide hydrochloride and of 2.10 ml of diisopropylethylamine. The mixture is stirred at ambient temperature for 18 h under N₂. After evaporation to dryness, the residue is taken up with ethyl acetate and H₂O. Extraction is carried out with ethyl acetate until the aqueous phase is completely depleted. The organic phase is washed with a saturated aqueous sodium chloride solution. After drying over MgSO₄ and concentration to dryness, the crude obtained is chromatographed, elution being carried out with a gradient of methanol in dichloromethane ranging from 0% to 10%. 5.29 g of tert-butyl [(1R)-1-(4-chlorobenzyl)-2-(trans-4-{cyclohexyl[(dimethylamino)carbonyl]amino}-3-methylpiperidin-1-yl)-2-oxoethyl]carbamate are obtained.

13.8: N-[trans-1-(4-chloro-D-phenylalanyl)-3-methylpiperidin-4-yl]1-N-cyclohexyl-N′,N′-dimethylurea

5.29 g of tert-butyl [(1R)-1-(4-chlorobenzyl)-2-(trans-4-{cyclohexyl[(dimethylamino)-carbonyl]amino}-3-methylpiperidin-1-yl)-2-oxoethyl]-carbamate are placed in 5 ml of dioxane. 24.1 ml of 4N hydrochloric acid in dioxane are then added. The reaction medium is stirred at ambient temperature for 18 h. After evaporation to dryness, the residue is taken up with dichloromethane and with a saturated aqueous sodium hydrogen carbonate solution. Extraction is carried out with dichloromethane until the aqueous phase is completely depleted. The organic phase is washed with H₂O and then with a saturated aqueous sodium chloride solution. After drying over MgSO₄ and concentration to dryness, 4.3 g of N-[trans-1-(4-chloro-D-phenylalanyl)-3-methylpiperidin-4-yl]-N-cyclohexyl-N′,N′-dimethylurea are obtained.

13.9: N-(cis-4-{[(1R)-1-(4-chlorobenzyl)-2-(trans-4-{cyclohexyl[(dimethylamino)carbonyl]amino}-3-methylpiperidin-1-yl)-2-oxoethyl]amino}cyclohexyl)-acetamide

0.5 g of N-[trans-1-(4-chloro-D-phenylalanyl)-3-methylpiperidin-4-yl]-N-cyclohexyl-N′,N′-dimethylurea, obtained in step 13.8, is dissolved in 11 ml of dichloromethane in the presence of 0.21 g of N-(4-oxocyclohexyl)acetamide, obtained in step 13.2. 0.35 g of sodium triacetoxyborohydride is then added under N₂. Stirring is maintained at ambient temperature for 18 h. After hydrolysis with a saturated aqueous sodium hydrogen carbonate solution, extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0.19 g and 0.34 g of N-(4-{[(1R)-1-(4-chlorobenzyl)-2-(trans-4-{cyclohexyl-[(dimethylamino)carbonyl]amino}-3-methylpiperidin-1-yl)-2-oxoethyl]amino}cyclohexyl)acetamide, mixture of stereoisomers of undetermined configuration, are obtained.

13.10: N-(4-{[(1R)-1-(4-chlorobenzyl)-2-(trans-4-{cyclohexyl[(dimethylamino)carbonyl]amino}-3-methylpiperidin-1-yl)-2-oxoethyl]amino}cyclohexyl)-acetamide hydrochloride

0.19 g of N-(4-{[(1R)-1-(4-chlorobenzyl)-2-(trans-4-{cyclohexyl[(dimethylamino)carbonyl]amino}-3-methylpiperidin-1-yl)-2-oxoethyl]amino}cyclohexyl)-acetamide is placed in 2 ml of ethyl acetate, and 0.16 ml of 2N hydrochloric acid in diethyl ether. After concentration to dryness, the reaction medium is taken up in ethyl diethyl ether and triturated. The precipitate obtained is then filter-dried and rinsed with diethyl ether. The hydrochloride thus obtained is dried over P₂O₅ under reduced pressure. 0.19 g of N-(4-{[(1R)-1-(4-chlorobenzyl)-2-(trans-4-{cyclohexyl-[(dimethylamino)carbonyl]amino}-3-methylpiperidin-1-yl)-2-oxoethyl]amino}cyclohexyl)acetamide hydrochloride is obtained.

Melting point=168° C.; M+H⁺=588

### EXAMPLE 14

### N-{(trans)-1-[4-chloro-N-(4-hydroxy-4-phenylcyclohexyl)-D-phenylalanyl]-3-methylpiperidin-4-yl}-N-cyclohexyl-N′,N′-dimethylurea hydrochloride (compound No. 110)

14.1: 4-phenyl-4-hydroxycyclohexanone

2.0 g of 1,4-cyclohexanedione are placed in 20 ml of diethyl ether and 40 ml of anhydrous tetrahydrofuran under N₂ at −78° C. 1.8N phenyl lithium in a cyclohexane/ether mixture is added slowly. Stirring of the medium is maintained at −78° C. for 2 h 20 min. After hydrolysis with a saturated aqueous ammonium chloride solution, the aqueous phase is extracted with ethyl acetate until said aqueous phase is completely depleted. The organic phase is washed with a saturated aqueous sodium chloride solution. After drying over MgSO₄ and concentration to dryness, the crude obtained is chromatographed on silica gel, elution being carried out with a mixture of cyclohexane/ethyl acetate ranging from 8/2 to 6/4. 0.64 g of 4-phenyl-4-hydroxycyclohexanone is obtained.

14.2: N-{(trans)-1-[4-chloro-N-(4-hydroxy-4-phenylcyclohexyl)-D-phenylalanyl]-3-methylpiperidin-4-yl}-N-cyclohexyl-N′,N′-dimethylurea 0.50 g of N-[trans-1-(4-chloro-D-phenylalanyl)-3-methylpiperidin-4-yl]-N-cyclohexyl-N′,N′-dimethylurea, obtained in step 13.8, is dissolved in 11.3 ml of dichloromethane in the presence of 0.25 g of 4-phenyl-4-hydroxycyclohexanone, obtained in step 14.1. 0.35 g of sodium triacetoxyborohydride is then added under N₂. Stirring is maintained at ambient temperature for 18 h. After the addition of 0.125 g of 4-phenyl-4-hydroxycyclohexanone and 0.175 g of sodium triacetoxyborohydride, stirring is maintained for 24 h. Hydrolysis is performed and extraction is carried out with dichloromethane until the aqueous phase is completely depleted. The organic phase is dried with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0.17 g of and 0.22 g of N-{(trans)-1-[4-chloro-N-(4-hydroxy-4-phenylcyclohexyl)-D-phenylalanyl]-3-methylpiperidin-4-yl}-N-cyclohexyl-N′,N′-dimethylurea, mixture of stereoisomers of undetermined configuration, are obtained.

14.3: N-{(trans)-1-[4-chloro-N-(4-hydroxy-4-phenylcyclohexyl)-D-phenylalanyl]-3-methylpiperidin-4-yl}-N-cyclohexyl-N′,N′-dimethylurea hydrochloride

0.22 g of N-{(trans)-1-[4-chloro-N-(4-hydroxy-4-phenylcyclohexyl)-D-phenylalanyl]-3-methylpiperidin-4-yl}-N-cyclohexyl-N′,N′-dimethylurea is placed in 2 ml of ethyl acetate, and 0.71 ml of 0.5N hydrochloric acid in diethyl ether is added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.20 g of N-{(trans)-1-[4-chloro-N-(4-hydroxy-4-phenylcyclohexyl)-D-phenylalanyl]-3-methylpiperidin-4-yl}-N-cyclohexyl-N′,N′-dimethylurea hydrochloride is obtained.

Melting point=194° C.; M+H⁺=623.

### EXAMPLE 15

### N-(trans-1-{4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea hydrochloride (compound No. 118)

15.1: 2-(1,4-dioxaspiro[4.5]dec-8-ylamino)-ethanol

3.12 g of 1,4-dioxaspiro[4.5]decan-8-one are dissolved in 80 ml of dichloromethane in the presence of 1.16 g of ethanolamine. 6.75 g of sodium triacetoxyborohydride are then added under N₂. Stirring is maintained at ambient temperature for 18 h. After hydrolysis with a 1N aqueous sodium hydroxide solution, extraction is carried out with dichloromethane until the aqueous phase is completely depleted. After drying over MgSO₄ and concentration to dryness, 4.0 g of 2-(1,4-dioxaspiro[4.5]dec-8-ylamino)ethanol are obtained, which product is subsequently used as it is.

15.2: 3-(1,4-dioxaspiro[4.5]dec-8-yl)-1,3-oxazolidin-2-one

1.47 g of disphosgene are placed in 50 ml of dichloromethane under N₂ and at 0° C. 1.0 g of 2-(1,4-dioxaspiro[4.5]dec-8-ylamino)ethanol, obtained in step 13.1, mixed with 3.59 ml of triethylamine, is added dropwise. Stirring is maintained at ambient temperature for 5 h. After evaporation to dryness, the crude obtained is taken up with dichloromethane. The organic phase is washed twice with a 1N aqueous hydrochloric acid solution, and then with H₂O and a saturated aqueous sodium chloride solution. After drying over MgSO₄ and concentration to dryness, the crude obtained is chromatographed on silica gel, elution being carried out with a gradient of methanol in dichloromethane ranging from 0% to 2%. 1.19 g of 3-(1,4-dioxaspiro-[4.5]dec-8-yl)-1,3-oxazolidin-2-one are obtained.

15.3: 3-(4-oxocyclohexyl)-1,3-oxazolidin-2-one

0.75 g of 3-(1,4-dioxaspiro[4.5]dec-8-yl)-1,3-oxazolidin-2-one is dissolved in 27.5 ml of 6N HCl. The reaction medium is heated at 65° C. for 5 h. After a return to ambient temperature, sodium carbonate is added slowly up to a pH of 9. Extraction is carried out with dichloromethane until the aqueous phase is completely depleted. The organic phase is washed with H₂O. After drying over MgSO₄, the crude obtained is chromatographed on silica gel, elution being carried out with a gradient of methanol in dichloromethane from 0% to 10%. 0.11 g of 3-(4-oxocyclohexyl)-1,3-oxazolidin-2-one is obtained.

15.4: N-((3S,4S)-1-{4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea 0.4 g of N-[trans-1-(4-chloro-D-phenylalanyl)-3-methylpiperidin-4-yl]-N-cyclohexyl-N′,N′-dimethylurea, obtained in step 13.8, is dissolved in 9 ml of dichloromethane in the presence of 0.20 g of 3-(4-oxocyclohexyl)-1,3-oxazolidin-2-one, obtained in step 15.3. 0.28 g of sodium triacetoxyborohydride is then added under N₂. Stirring is maintained at ambient temperature for 18 h. Hydrolysis is performed with a saturated aqueous sodium hydrogen carbonate solution, and extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/acetone/methanol ranging from 100/0/0 to 70/25/5. 0.21 g of and 0.19 g of N-((3S,4S)-1-(4-chloro-N-[4-(2-oxo-1,3-oxazolidin-3-yl)cyclohexyl]-D-phenylalanyl]-3-methylpiperidin-4-yl)]-N-cyclohexyl-N′,N′-dimethylurea, mixture of stereoisomers of undetermined configuration, are obtained.

15.5: N-(trans-1-[4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea hydrochloride

0.21 g of N-(trans-1-{4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea is placed in 2 ml of ethyl acetate, and 1.7 ml of 0.2N hydrochloric acid in diethyl ether are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.18 g of N-(trans-1-[(4-chloro-N-[4-(2-oxo-1,3-oxazolidin-3-yl)cyclohexyl]-D-phenylalanyl]-3-methylpiperidin-4-yl)-N-cyclohexyl-N′,N′-dimethylurea hydrochloride is obtained.

Melting point=189° C.; M+H⁺=616;

### EXAMPLE 16

### N-{trans-1-[4-chloro-N-(1-isonicotinoylpiperidin-4-yl)-D-phenylalanyl]-3-methylpiperidin-4-yl}-N-cyclohexyl-N′,N′-dimethylurea hydrochloride (compound No. 119)

16.1: 8-isonicotinoyl-1,4-dioxa-8-azaspiro[4.5]decane

1.34 ml of 1,4-dioxaspiro[4.5]decan-8-one are dissolved in 104 ml of dichloromethane in the presence of 1.4 g of isonicotinic acid, of 1.56 g of hydroxybenzotriazole, of 2.21 g of 1-(3-dimethylaminopropyl)-3-ethylcarbodiimide hydrochloride and of 1.49 ml of diisopropylethylamine. The mixture is stirred at ambient temperature for 18 h under N₂. After evaporation to dryness, the residue is hydrolysed with a 1N aqueous sodium hydroxide solution. Exraction is carried out with dichloromethane until the aqueous phase is completely depleted. After drying over Na₂SO₄ and concentration to dryness, the crude obtained is chromatographed, elution being carried out with an 85/5 mixture of dichloromethane/methanol. 2.63 g of 8-isonicotinoyl-1,4-dioxa-8-azaspiro[4.5]decane are obtained.

16.2: 1-isonicotinoylpiperidin-4-one

2.6 g of 8-isonicotinoyl-1,4-dioxa-8-azaspiro[4.5]decane are dissolved in 43 ml of 6N HCl. The reaction medium is heated at 65° C. for 18 h. The reaction medium is placed at 0° C. and sodium carbonate is added slowly up to a pH of 9. Extraction is carried out with dichloromethane until the aqueous phase is completely depleted. After drying over Na₂SO₄, the crude obtained is chromatographed on silica gel, elution being carried out with a gradient of methanol in dichloromethane from 0% to 10%. 0.19 g of 1-isonicotinoylpiperidin-4-one is obtained.

16.3: N-{trans-1-[4-chloro-N-(1-isonicotinoylpiperidin-4-yl)-D-phenylalanyl]-3-methylpiperidin-4-yl}-N-cyclohexyl-N′,N′-dimethylurea

0.3 g of N-[trans-1-(4-chloro-D-phenylalanyl)-3-methylpiperidin-4-yl]-N-cyclohexyl-N′,N′-dimethylurea, obtained in step 13.8, is dissolved in 7 ml of dichloromethane in the presence of 0.18 g of 1-isonicotinoylpiperidin-4-one, obtained in step 16.3. 0.21 g of sodium triacetoxyborohydride is then added under N₂. Stirring is maintained at ambient temperature for 18 h. Hydrolysis is performed with a saturated aqueous sodium hydrogen carbonate solution, and extraction is carried out with dichloromethane until the aqueous phase is completely depleted. The organic phase is washed with H₂O and then with a saturated aqueous sodium chloride solution. After drying over MgSO₄ and concentration to dryness, the crude obtained is chromatographed on silica gel, elution being carried out with a mixture of dichloromethane/methanol/aqueous ammonia ranging from 100/0/0 to 90/10/1. 0.075 g of and 0.23 g of N-{trans-1-[4-chloro-N-(1-isonicotinoylpiperidin-4-yl)-D-phenylalanyl]-3-methylpiperidin-4-yl}-N-cyclohexyl-N′,N′-dimethylurea, mixture of diastereoisomers of undetermined configuration, are obtained.

16.4: N-{trans-1-[4-chloro-N-(1-isonicotinoylpiperidin-4-yl)-D-phenylalanyl]-3-methylpiperidin-4-yl}-N-cyclohexyl-N′,N′-dimethylurea

0.23 g of N-{trans-1-[4-chloro-N-(1-isonicotinoylpiperidin-4-yl)-D-phenylalanyl]-3-methylpiperidin-4-yl}-N-cyclohexyl-N′,N′-dimethylurea is placed in 2 ml of ethyl acetate, and 1.8 ml of 0.2N hydrochloric acid in diethyl ether are added. After concentration to dryness, the reaction medium is taken up with diethyl ether and triturated. The precipitate obtained is then filter-dried and rinsed with diethyl ether. The hydrochloride thus obtained is dried over P₂O₅ under reduced pressure. 0.18 g of N-(trans-1-[4-chloro-N-(1-isonicotinoylpiperidin-4-yl)-D-phenylalanyl]-3-methylpiperidin-4-yl}-N-cyclohexyl-N′,N′-dimethylurea hydrochloride is obtained.

Melting point=206° C.; M+H⁺=640.

The table that follows illustrates the chemical structures and the physical properties of some examples of compounds according to the invention, i.e. of the compounds of formula (Ia), corresponding to compounds of formula (I) in which R_{a′}=R₅=H, and R₃ represents a chlorine atom located in the para-position on the phenyl ring to which it is attached.

In this table:
the carbon atom bearing the 4-Cl-benzyl group has the (R) configuration,
in the “salt” column, “—” represents a compound in the form of a free base, whereas “HCl” represents a compound in the form of a hydrochloride and “CF₃COOH” represents a compound in the form of a trifluoroacetate,
when Ra is a methyl group, the compound is obtained in the form of a mixture of diastereoisomers,
“Mp” represents the melting point of the compound, and

Me, Et, and iPr represent, respectively, methyl, ethyl, and isopropyl groups.

TABLE

| | |
|---|---|
| | (Ia) |
| 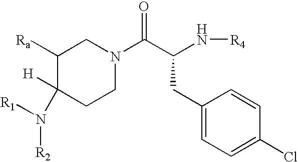 | |

| No. | Rₐ | R₁ | R₂ | R₄ | salt | Mp (° C.) |
|---|---|---|---|---|---|---|
| 1 | H | cyclohexyl | —CO—N(Et)₂ | 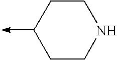 | HCl | >220 |
| 2 | H | cyclohexyl | —CO—N(Et)₂ | 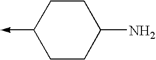 | HCl | 195 |
| 3 | H | cyclohexyl | —CO—N(Et)₂ | 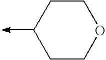 | HCl | >200 |
| 4 | H | cyclohexyl | —CO—N(Et)₂ | 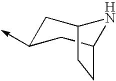 | HCl | 182 |
| 5 | H | cyclohexyl | —CO—N(Et)₂ | 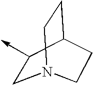 | HCl | 169 |
| 6 | H | cyclohexyl | —CO—N(Et)₂ | 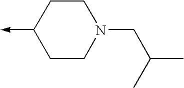 | HCl | >250 |
| 7 | H | cyclohexyl | 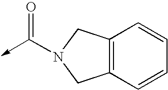 | 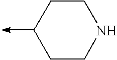 | HCl | 220 |
| 8 | H | cyclohexyl | 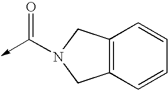 | 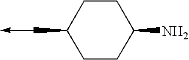 | HCl | 230 |
| 9 | H | cyclohexyl | 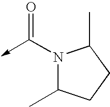 | 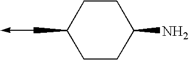 | HCl | 110 |
| 10 | H | cyclohexyl | 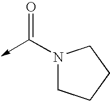 | 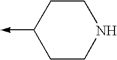 | HCl | 256 |
| 11 | H | cyclohexyl | —CO—N(Me)₂ | 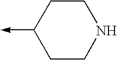 | HCl | 270 |
| 12 | H | cyclohexyl | —CO—N(Me)₂ | 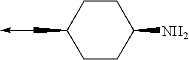 | HCl | 139 |
| 13 | H | cyclohexyl | 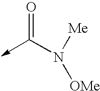 | 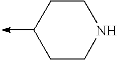 | HCl | 254 |
| 14 | H | cyclohexyl | 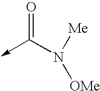 | 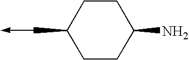 | HCl | 119 |
| 15 | H | cyclohexyl | 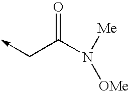 | 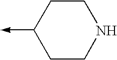 | HCl | 182 |
| 16 | H | cyclohexyl | 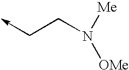 | 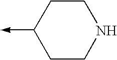 | HCl | 166 |
| 17 | H | cyclohexyl | 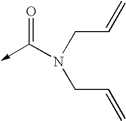 | 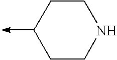 | HCl | 258 |
| 18 | H | cyclohexyl | 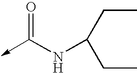 | 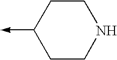 | HCl | 178 |
| 19 | H | cyclohexyl | 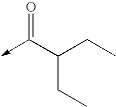 | 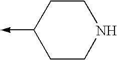 | HCl | 285 |
| 20 | H | cyclohexyl | 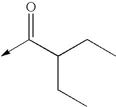 | 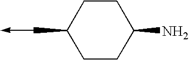 | HCl | 230 |
| 21 | H | cyclohexyl | 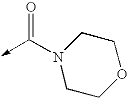 | 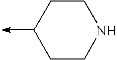 | HCl | 279 |
| 22 | H | cyclohexyl | 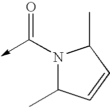 | 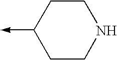 | HCl | 252 |
| 23 | H | cyclohexyl | 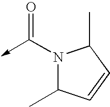 | 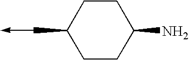 | HCl | 112 |
| 24 | H | cyclohexyl | 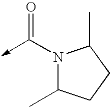 | 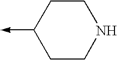 | HCl | 257 |
| 25 | H | cyclohexyl | 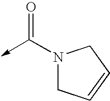 | 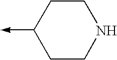 | HCl | 244 |
| 26 | H | cycloheptyl | —CO—N(Et)₂ | 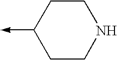 | HCl | 238 |
| 27 | H | cycloheptyl | —CO—N(Et)₂ | 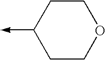 | HCl | >250 |
| 28 | H | cycloheptyl | —CO—N(Et)₂ | 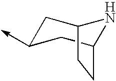 | HCl | 208 |
| 29 | H | cyclobutyl | —CO—N(Et)₂ | 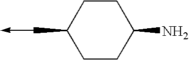 | HCl | 138 |
| 30 | H | cyclobutyl | —CO—N(Et)₂ | 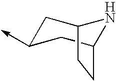 | HCl | 225 |
| 31 | H | cyclopentyl | —CO—N(Et)₂ | 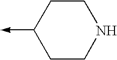 | HCl | >250 |
| 32 | H | cyclopentyl | —CO—N(Et)₂ | 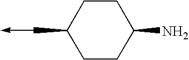 | HCl | 200 |
| 33 | H | cycloheptyl | —CO—N(Et)₂ | 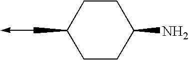 | HCl | 105 |
| 34 | H | cyclooctyl | —CO—N(Et)₂ | 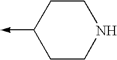 | HCl | 190 |
| 35 | H | cyclooctyl | —CO—N(Et)₂ | 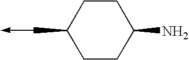 | HCl | 128 |
| 36 | H | phenyl | —CO—N(Et)₂ | 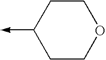 | HCl | >250 |
| 37 | H | phenyl | —CO—N(Et)₂ | 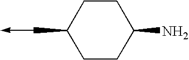 | HCl | 123 |
| 38 | H | cyclohexyl | —CONH—CH₂CF₃ | 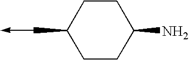 | HCl | 230 |
| 39 | H | cyclohexyl | 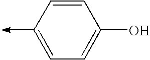 | 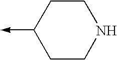 | HCl | 215 |
| 40 | H | cyclohexyl | 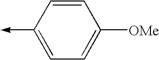 | 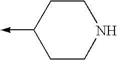 | HCl | 195 |
| 41 | H | cyclohexyl | 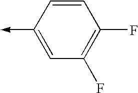 | 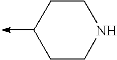 | HCl | 203 |
| 42 | H | cyclohexyl | 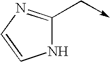 | 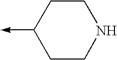 | Fumaric acid | 122 |
| 43 | H | cyclohexyl | 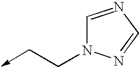 | 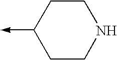 | HCl | 184 |
| 44 | H | cyclohexyl | 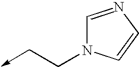 | 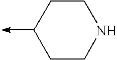 | HCl | 162 |
| 45 | H | cyclohexyl | —CO—N(Et)₂ | 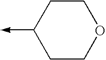 | HCl | >200 |
| 46 | Me (trans) | cyclohexyl | —CO—N(Et)₂ | 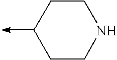 | HCl | 220 |
| 47 | Me (cis) | cyclohexyl | —CO—N(Et)₂ | 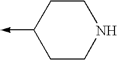 | HCl | 196 |
| 48 | Me (trans) | cyclohexyl | —CO—N(Et)₂ | 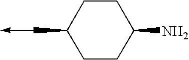 | HCl | 115 |
| 49 | Me (cis) | cyclohexyl | —CO—N(Et)₂ | 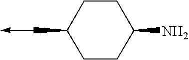 | HCl | 110 |
| 50 | Me (trans) | cyclohexyl | —CO—N(Et)₂ | 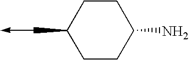 | HCl | 238 |
| 51 | Me (cis) | cyclohexyl | —CO—N(Et)₂ | 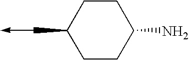 | HCl | 220 |
| 52 | Me (trans) | cyclohexyl | —CO—N(Et)₂ | 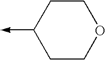 | HCl | 145 |
| 53 | Me (cis) | cyclohexyl | —CO—N(Et)₂ | 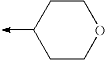 | HCl | 135 |
| 54 | H | H | —CO—N(Et)₂ | 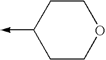 | HCl | 190 |
| 55 | H | cyclohexyl | 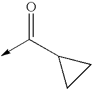 | 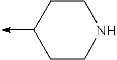 | HCl | >240 |
| 56 | H | cyclohexyl | 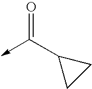 | 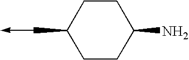 | HCl | 240 |
| 57 | H | cyclohexyl | 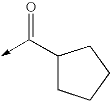 | 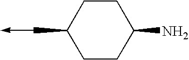 | HCl | 240 |
| 58 | H | cyclohexyl | 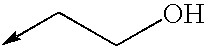 | 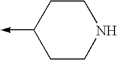 | HCl | >200 |
| 59 | H | 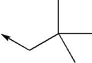 | —CO—N(Et)₂ | 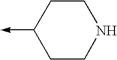 | HCl | 285 |
| 60 | H | 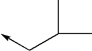 | —CO—N(Et)₂ | 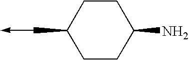 | HCl | 219 |
| 61 | H | 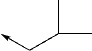 | —CO—N(Et)₂ | 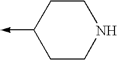 | HCl | >250 |
| 62 | H | cyclohexyl | 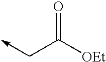 | 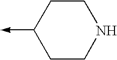 | HCl | 215 |
| 63 | H | 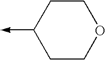 | —CO—N(Et)₂ | 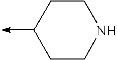 | HCl | >240 |
| 64 | H | 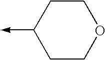 | —CO—N(Et)₂ | 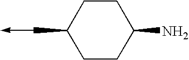 | HCl | 87 |
| 65 | H | 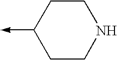 | —CO—N(Et)₂ | 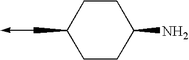 | HCl | >250 |
| 66 | H | cyclohexyl | —CO—N(Et) (iPr) | 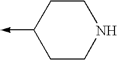 | HCl | >240 |
| 67 | H | cyclohexyl | —CO—N(Et) (iPr) | 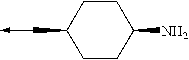 | HCl | 171 |
| 68 | H | cyclohexyl | —CO—N(Et)₂ | 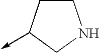 | HCl | 110 |
| 69 | H | cyclohexyl | 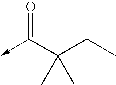 | 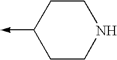 | HCl | >240 |
| 70 | H | cyclohexyl | 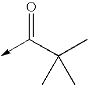 | 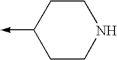 | HCl | >240 |
| 71 | H | cyclohexyl | 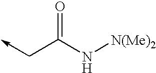 | 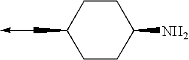 | HCl | 124 |
| 72 | H | cyclohexyl | —CO—N(iPr)₂ | 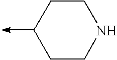 | HCl | 189 |
| 73 | H | cyclohexyl | 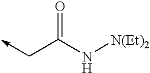 | 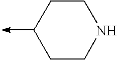 | HCl | 88 |
| 74 | H | cyclohexyl | —CO—N(Et)₂ | 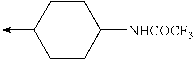 | HCl | 144 or 173** |
| 75 | H | cyclohexyl | —CO—N(Et)₂ | 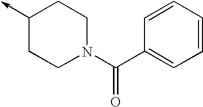 | HCl | >230 |
| 76 | H | cyclohexyl | 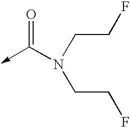 | 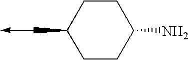 | HCl | 84 |
| 77 | H | cyclohexyl | 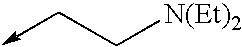 | 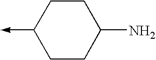 | HCl | 88 |
| 78 | H | cyclohexyl | 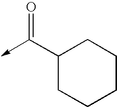 | 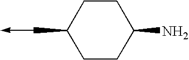 | HCl | 250 |
| 79 | H | cyclohexyl | 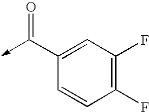 | 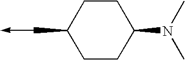 | HCl | 113 |
| 80 | H | cycloheptyl | —CO—N(Me)₂ | 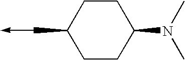 | HCl | 235 |
| 81 | H | cyclohexyl | 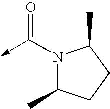 | 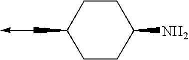 | HCl | 131 |
| 82 | H | cyclohexyl | 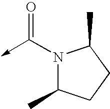 | 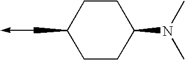 | HCl | 78 |
| 83 | H | cyclohexyl | —CO—N(Me)₂ | 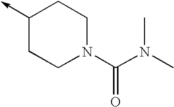 | HCl | 256 |
| 84 | H | cyclohexyl | —CO—N(Me)₂ | 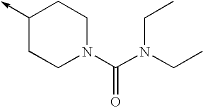 | HCl | 265 |
| 85 | H | cyclohexyl | —CO—N(Me)₂ | 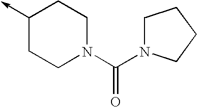 | HCl | 264 |
| 86 | H | cyclohexyl | —CO—N(Me)₂ | 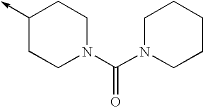 | HCl | 275 |
| 87 | H | cyclohexyl | —CO—N(Me)₂ | 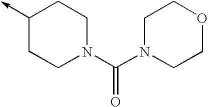 | HCl | 256 |
| 88 | H | cyclohexyl | —CO—N(Me)₂ | 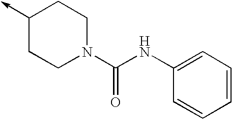 | HCl | 164 |
| 89 | H | cyclohexyl | —CO—N(Me)₂ | 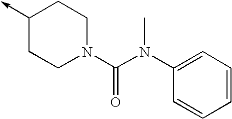 | HCl | 274 |
| 90 | H | cyclohexyl | —CO—N(Me)₂ | 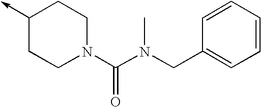 | HCl | 251 |
| 91 | H | cyclohexyl | —CO—N(Me)₂ | 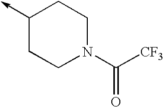 | HCl | 261 |
| 92 | H | cyclohexyl | —CO—N(Me)₂ | 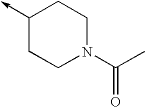 | HCl | 251 |
| 93* | H | cyclohexyl | —CO—N(Me)₂ | 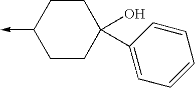 | HCl | 210 or 240** |
| 94* | H | cyclohexyl | —CO—N(Me)₂ | 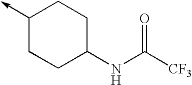 | HCl | 170 or 190** |
| 95* | H | cyclohexyl | —CO—N(Me)₂ | 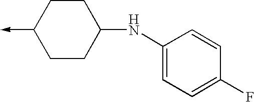 | HCl | 180 or 230** |
| 96 | H | cyclohexyl | —CO—N(Me)₂ | 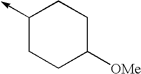 | HCl | 260 |
| 97* | H | cyclohexyl | —CO—N(Me)₂ | 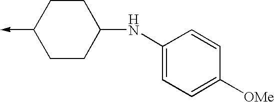 | HCl | 175 or 193** |
| 98* | H | cyclohexyl | —CO—N(Me)₂ | 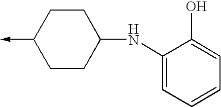 | HCl | 180 or 185** |
| 99 | H | cyclohexyl | —CO—N(Me)₂ | 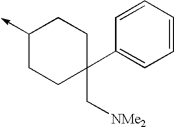 | HCl | 250 |
| 100 | H | cyclohexyl | 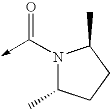 | 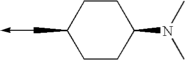 | HCl | 187 |
| 101 | H | cyclohexyl | 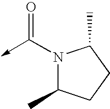 | 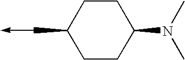 | HCl | 220 |
| 102 | Me (trans) | cyclohexyl | —CO—N(Me)₂ | 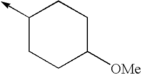 | HCl | 235 |
| 103 | Me (trans) | cyclohexyl | —CO—N(Me)₂ | 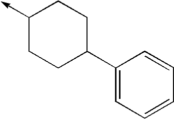 | HCl | 182 or 198** |
| 104 | Me (trans) | cyclohexyl | —CO—N(Me)₂ | 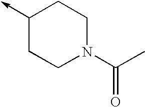 | HCl | 169 |
| 105 | Me (trans) | cyclohexyl | —CO—N(Me)₂ | 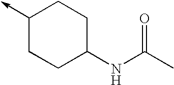 | HCl | 168 or 243** |
| 106 | Me (trans) | cyclohexyl | —CO—N(Me)₂ | 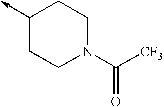 | HCl | 185 |
| 107 | Me (trans) | cyclohexyl | —CO—N(Me)₂ | 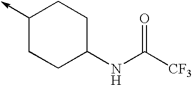 | HCl | 188 or 229* |
| 108 | Me (trans) | cyclohexyl | —CO—N(Me)₂ | 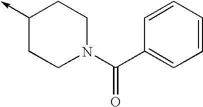 | HCl | 187 |
| 109 | Me (trans) | cyclohexyl | —CO—N(Me)₂ | 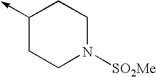 | HCl | 150 197** |
| 110 | Me (trans) | cyclohexyl | —CO—N(Me)₂ | 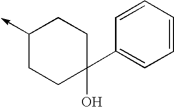 | HCl | 165 or 194** |
| 111 | Me (trans) | cyclohexyl | —CO—N(Me)₂ | 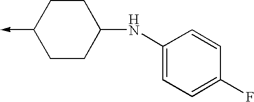 | HCl | 213 |
| 112 | H | cyclohexyl | —CO—N(Me)₂ | 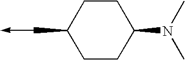 | HCl | 92 |
| 113 | Me (trans) | cyclohexyl | —CO—N(Me)₂ | 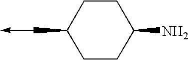 | HCl | 98 or 125** |
| 114 | H | cyclohexyl | —CO—N(Me)₂ | 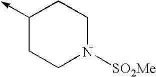 | HCl | 206 |
| 115* | H | cyclohexyl | —CO—N(Me)₂ | 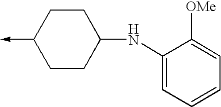 | HCl | 133 or 153** |
| 116* | H | cyclohexyl | —CO—N(Me)₂ | 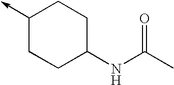 | HCl | 131 or 133* |
| 117* | H | cyclohexyl | —CO—N(Me)₂ | 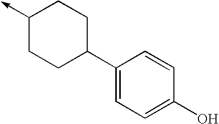 | HCl | 221 or 235** |
| 118 | Me (trans) | cyclohexyl | —CO—N(Me)₂ | 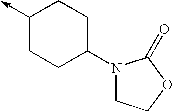 | HCl | 189 or 206** |
| 119 | Me (trans) | cyclohexyl | —CO—N(Me)₂ | 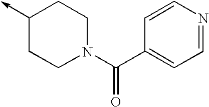 | HCl | 206 |
| 120 | Me (trans) | cyclohexyl | —CO—N(Me)₂ | 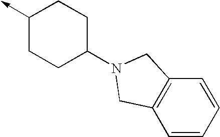 | HCl | 142 or 211** |
| 121 | Me (trans) | cyclohexyl | —CO—N(Me)₂ | 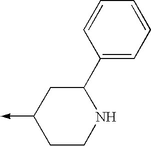 | HCl | 211 |
| 122 | Me (trans) | cyclohexyl | —CO—N(Me)₂ | 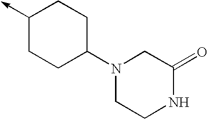 | HCl | 133 207** |

*according to the isomer (cis or trans)
**according to the mixtures of diastereoisomers

The compounds according to the invention were the subject of pharmacological assays to determine their melanocortin receptor agonist effect, in particular their MC3 and/or MC4 receptor agonist effect.

Evaluation of the Affinity of the Compounds of Formula (I) According to the Invention with respect to MC3 and MC4 Receptors

This affinity assay is carried out by measuring the binding of [¹²⁵I]-[Nle⁴-D-Phe⁷]-□-MSH to cell membranes. The displacement of this radioligand is used to identify inhibitors of the specific binding to recombinant melanocortin receptors.

For this assay, membranes prepared from CHO-K1 cells expressing the human MC4 receptor at high density (Euroscreen) or membranes, that were purchased (Perkin Elmer Life Sciences, Receptor Biology), of HEK-293 cells expressing hMC3 receptors were used. CHO-K1 cells transfected with the hMC4 receptor gene (Euroscreen) are seeded into DMEM/Nutrient Mix F12 culture medium containing 10% foetal calf serum (Biowhittaker), 1% sodium pyruvate, 1% L-glutamine, 1% non-essential amino acids, 0.4 mg/ml geneticin (G418) and 0.5% PenStrep, these products being provided by Gibco/BR1, except the calf serum. At 80% confluency, the cells are scraped off and the cell pellets are frozen at −80° C.

A tube of cells (approximately 70×10⁶ cells) is thawed on ice and resuspended in 10 ml of binding buffer [25 mM HEPES, pH 7.0, 1 mM MgCl₂, 1.5 mM CaCl₂, 100 mM NaCl, 1 mM 1,10-phenanthroline and 1 tablet of Complete^{TR} (protease inhibitor from Roche) in 50 ml of buffer] using a polytron for 20 seconds. The suspension is centrifuged for 20 min at 19 500 rpm at 4° C. The supernatant is discarded and the pellet is resuspended in 5 ml of binding buffer. The amount of proteins present in the sample is assayed using a Bradford test, and the concentration is adjusted to 3 μg/25 μl by dilution in binding buffer.

[¹²⁵I]-[Nle⁴, D-Phe⁷]-□-MSH is diluted in binding buffer+0.2% BSA. SPA beads (wheatgerm agglutinin polyvinyltoluene, Amersham Pharmacia Biotech) are hydrated in the binding buffer+0.2% BSA and are then mixed with the cell homogenate so as to obtain 3 μg of cell proteins and 250 μg of beads in 50 μl. The products to be tested (diluted in 10% DMSO), in an amount of 10 μl at a concentration of 10 times the final concentration, are distributed into a clear-bottomed 96-well white plate (CORNING 3604 Polystyrene Non-Binding Surface). The nonspecific binding is defined by NDP-□MSH at 10⁻⁷ M. The total binding is measured by the number of counts per minute in the presence of the radioligand alone. The distribution of the membranes-beads suspension (50 μl/well) is followed by distribution of the solution of [¹²⁵I]-[Nle⁴, D-Phe⁷]-□-MSH, 40 μl/well (final concentration of 100 pM), for a final volume of 100 μl/well. After incubation at ambient temperature for 6 h, counting is carried out in a Microbeta TriLux scintillation counter. The IC₅₀ value for the compounds corresponds to the concentration that displaces the specific binding of the radioligand by 50%.

It is thus determined that the compounds according to the invention exhibit affinity for MC3 and/or MC4 receptors. Their IC₅₀ values with respect to MC3 and MC4 receptors are less than 10 μM, and for most of them between 1 nM and 1 μM. As examples, compound No. 2 of the table exhibits an IC₅₀ of 300 nM with respect to the MC4 receptor.

Evaluation of the Agonist Activity of the Compounds of Formula (I) According to the Invention, with Respect to MC3 and MC4 Receptors

A functional assay is used to differentiate between the agonist activity and the antagonist activity. For this, the formation of cyclic adenosine monophosphate (cAMP) generated by activation of the MC3 receptor or of the MC4 receptor is assayed.

CHO-K1 cells, expressing the human MC4 receptor at a moderate density (Euroscreen), are seeded into DMEM/Nutrient Mix F12 culture medium (Gibco/BR1) containing 10% of foetal calf serum, 0.5% sodium pyruvate, 1% L-glutamine, 1% non-essential amino acids, 200 mg/l hygromycin B and 0.5% PenStrep, these products being provided by Gibco/BR1, except the calf serum (Biowhittaker) and hygromycin B (Sigma).

CHO(dhfr-) cells expressing the human MC3 receptor are seeded into MEM Eagle culture medium (Sigma) containing 10% of dialysed calf serum, 1% L-glutamine, 1% sodium pyruvate, 20 mg/500 ml L-proline, 0.3 mg/ml Geneticin and 0.5% PenStrep, these products being provided by Gibco/BR1, except for the dialysed calf serum (Cambrex) and the L-proline (Sigma).

The compounds to be tested (diluted in 10% DMSO), in an amount of 10 μl at a concentration of 10 times the final concentration, are added to the plates of cells (final volume=100 μl/well). After incubation for 1 hour (37° C., 5% CO₂), the amount of cAMP is assayed using Tropix kits (Appelera) according to the supplier's documentation. The intrinsic activity of the compounds is calculated by comparing the stimulation of cAMP by these compounds to the stimulation induced by 30 nM of NDP□MSH (maximum of 100%). The EC₅₀ value for the compounds corresponds to the concentration which produces 50% of the maximum stimulation obtained with this compound.

It is thus determined that the compounds according to the invention are MC3- and/or MC4-receptor agonists. They have EC₅₀ values with respect to MC3 and MC4 receptors of less than 10 μM, and for most of them of between 1 nM and 1 μM. As examples, compounds No. 1 and 2 of the table have, respectively, EC₅₀ values of 590 nM and 370 nM with respect to the MC3 receptor, and of 80 nM and 30 nM with respect to the MC4 receptor.

As the compounds according to the invention exhibit melanocortin receptor agonist activity, they can therefore be used in the manufacture of medicaments. Thus, according to another of its aspects, a subject of the invention is medicaments which comprise a compound of formula (I), or an addition salt of the latter with a pharmaceutically acceptable acid, or else a hydrate or a solvate of the compound of formula (I).

These medicaments find their use in therapeutics, in pathologies in which melanocortin receptors, in particular MC3 and/or MC4 receptors, are involved: this involves in particular the treatment and prevention of obesity, diabetes and sexual dysfunctions that can affect both sexes, such as erectile dysfunctions, cardiovascular diseases such as myocardial infarction or hypertension, and also in anti-inflammatory uses or in the treatment of alcohol dependency.

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt, or a hydrate or a solvate of said compound, and also at least one pharmaceutically acceptable excipient. Said excipients are chosen, according to the pharmaceutical form and the method of administration desired, from the usual excipients that are known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or its possible salt, solvate or hydrate, can be administered in unit administration form, as a mixture with conventional pharmaceutical excipients, to animals and to human beings for the prophylaxis or the treatment of the conditions or of the diseases above.

Suitable unit administration forms comprise oral forms such as tablets, soft or hard gelatin capsules, powders, granules and oral solutions or suspensions, sublingual, buccal, intratracheal, intraocular or intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

A preferred administration form is oral administration.

By way of example, a unit administration form of a compound according to the invention in the form of a tablet can comprise the following constituents:

| | | |
|---|---|---|
| Compound according to the invention | 50.0 | mg |
| Mannitol | 223.75 | mg |
| Sodium croscaramellose | 6.0 | mg |
| Corn starch | 15.0 | mg |
| Hydroxypropylmethylcellulose | 2.25 | mg |
| Magnesium stearate | 3.0 | mg |

There may be specific cases where higher or lower dosages are appropriate; such dosages do not depart from the context of the invention. According to the usual practice, the dosage appropriate for each patient is determined by the physician according to the method of administration, and the weight and response of said patient.

According to another of its aspects, the present invention also relates to a method of treating the pathologies indicated above, which comprises the administration, to a patient, of an effective dose of a compound according to the invention, or one of its pharmaceutically acceptable salts or hydrates or solvates.

1. A compound corresponding to formula (I): 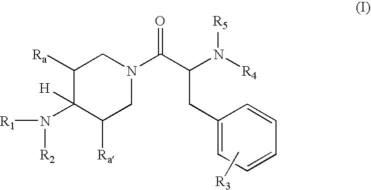
in which:
Rₐ and R_{a′}, which may be identical to or different from one another, represent a hydrogen atom, or an alkyl or cycloalkyl group,
R₁ represents a hydrogen atom, or an alkyl cycloalkyl, heteroaryl or aryl group,
R₂ represents a group, of formula —(CH₂)ₙ—(CO)_{y}—Y or —(CO)_{y}—(CH₂)ₙ—Y, in which:
x=0, 1, 2, 3 or 4,
y=0 or 1,
Y represents a hydrogen atom, or a hydroxyl, alkyl, cycloalkyl, alkoxy, aryl, heteroaryl or —NR₁₁R₁₂ group, Y being different from a hydrogen atom when x=y=0,
R₁₁ and R₁₂, which may be identical to or different from one another, represent a hydrogen atom, or an alkyl, cycloalkyl, alkoxy or —NR₁₃R₁₄ group, or else R₁₁ and R₁₂ form, together with the nitrogen atom to which they are attached, a mono- or bicyclic structure containing from 4 to 10 ring members and optionally comprising 1 to 3 additional hetero atoms and/or 1 to 3 ethylenic or acetylenic unsaturations, this ring being optionally substituted in any positions with 1 to 3 groups chosen from halogen atoms, and hydroxyl, alkyl, cycloalkyl and alkoxy groups,
R₁₃ and R₁₄, which may be identical to or different from one another, represent a hydrogen atom, or an alkyl, cycloalkyl or alkoxy group, or else R₁₃ and R₁₄ form, together with the nitrogen atom to which they are attached, a mono- or bicyclic structure as defined above,
R₃ represents 1 to 3 groups, which may be identical to or different from one another, located in any positions of the ring to which they are attached and chosen from halogen atoms, and alkyl, cycloalkyl, —OR, —NRR′, —CO—NRR′, —NR—CO—R′, —NR—CO—NRR′, —NR—COOR′, —NO₂, —CN and —COOR groups,
R₅ represents a hydrogen atom or an alkyl or cycloalkyl group,
R₄ is chosen from the groups of formulae (a), (b) and (c), optionally substituted with an oxo group or mono- or polysubstituted with an aryl or heteroaryl group, below: 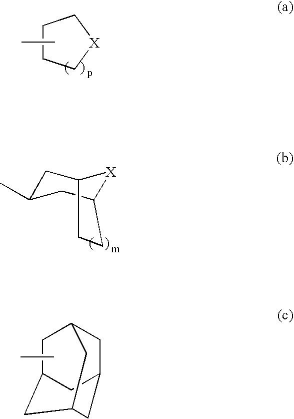
in which:
p=0, 1, 2 or 3,
m=0, 1 or 2,
and either
a) X represents a ring member —N(R₁₀)—, where
R₁₀ is chosen from:
a group —(CH₂)ₓ—OR₈, —(CH₂)ₓ—COOR₈, —(CH₂)ₓ—NR₈R₉, —(CH₂)ₓ—CO—NR₈R₉ or —(CH₂)ₓ—NR₈—COR₉, —(CH₂)ₓ—COR₈ in which x=1, 2, 3 or 4,
a cycloalkyl or heterocycloalkyl group fused with an aryl or heteroaryl group,
a cycloalkyl, heterocycloalkyl, aryl, hetero-aryl, alkylaryl, alkylheteroaryl, —CO-alkyl, —CO-cyclo-alkyl, —CO-heterocycloalkyl, —CO-aryl, —CO-heteroaryl, —CO-alkylaryl, —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉, —C (═NH)—NR₈R₉, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl or —SO₂—NR₈R₉ group,
the alkyl, cycloalkyl, heterocycloalkyl, aryl or heteroaryl groups being optionally substituted with 1 or more groups chosen from the groups R, R′, —OR, —NRR′, —CO—NRR′, —NR—CO—R′, —NR—CO—NRR′, —NO₂, —CN and —COOR, OCOR, COR, OCONRR′, NRCOOR′
the cycloalkyl or heterocycloalkyl groups being optionally fused with an aryl or heteroaryl group;
or else R₁₀ forms, with the nitrogen atom to which it is attached and a carbon atom located in any position of the cyclic structure of formula (a), but not adjacent to said nitrogen atom, a bridge comprising from 3 to 5 members,
R₈ and R₉ are chosen, independently of one another, from a hydrogen atom, and alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4,
or else R₈ and R₉ together form a cycloalkyl or a heterocycloalkyl;
R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl; or,
b) X represents a ring member —C(R₆)(R₇)—, where
R₆ is chosen from:
a hydrogen atom, a halogen atom,
a group —(CH₂)ₓ—OR₈, —(CH₂)ₓ—COOR₈, —(CH₂)—NR₈R₉, —(CH₂)ₓ—CO—NR₈R₉ or —(CH₂)ₓ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉ or —C(═NH)—NR₈R₉ group,
a fused or nonfused cycloalkyl or heterocycloalkyl group located in the spiro position on the ring of formula (a) to which it is attached,
a cycloalkyl or heterocycloalkyl group fused with an aryl or heteroaryl group,
the alkyl, cycloalkyl, heterocycloalkyl, aryl or heteroaryl groups being optionally substituted with 1 or more groups chosen from the groups R, R′, —OR, —NRR′, —CO—NRR′, —NR—CO—R′, —NR—CO—NRR′, —NO₂, —CN and —COOR, OCOR, COR, OCONRR′, NRCOOR′;
the cycloalkyl or heterocycloalkyl groups being optionally fused with an aryl or heteroaryl group,
R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkylaryl, —O-alkylheteroaryl, —NRR′, —CO—NRR′, —NR—CO—R′, —NR—CO—NRR′, —NR—COOR′, —NO₂, —CN and —COOR groups,
R₈ and R₉ are chosen, independently of one another, from a hydrogen atom, and alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C (═N—H)—NRR′, —COOR, —CO—NRR′, —CS—NRR′ and —(CH₂)ₙ—OR groups, where x=0, 1, 2, 3 or 4, the alkyl and aryl groups being optionally substituted with one or more groups chosen from the groups R, R′, —OR, —NRR′, —CO—NRR′, —NR—CO—R′, —NR—CO—NRR′, —NO₂, —CN and —COOR, OCOR, COR, OCONRR′, NRCOOR′;
or else R₈ and R₉ together form a cycloalkyl or a heterocycloalkyl;
R and R′ represent, independently of one another, a hydrogen atom, or an alkyl, cycloalkyl, heterocycloalkyl, aryl, heteroaryl, alkylaryl or alkylheteroaryl group, or can together form a cycloalkyl or a heterocycloalkyl,
in the form of a base or of an addition salt with an acid, and also in the form of a hydrate or of a solvate.

2. A compound according to claim 1, wherein R₄ is chosen from the groups of formulae (a), (b) and (c), optionally mono- or polysubstituted with an aryl or heteroaryl group, where X represents a ring member —C(R₆) (R₇)—, in which R₆ is chosen from:
a hydrogen atom,
a group —(CH₂) x-OR₈, —(CH₂) X—COOR₈, —(CH₂)ₓ—NR₈R₉, —(CH₂)ₓ—CO—NR₈R₉ or —(CH₂)ₓ—NR₈—COR₉, in which x=0, 1, 2, 3 or 4,
an alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl or —CO-alkylheteroaryl group,
a cycloalkyl or heterocycloalkyl group located in the spiro position on the ring of formula (a) to which it is attached,
a cycloalkyl or heterocycloalkyl group fused with an aryl or heteroaryl group,
R₇ is chosen from hydrogen and halogen atoms, and alkyl, cycloalkyl, aryl, heteroaryl, alkylaryl, alkylheteroaryl, —OR, —O-aryl, —O-heteroaryl, —O-alkyl-aryl, —O-alkylheteroaryl, —NRR′, —CO—NRR′, —NR—CO—R′, —NR—CO—NRR′, —NR—COOR′, —NO₂, —CN and —COOR groups,
R₈ and R₉ are chosen, independently of one another, from a hydrogen atom, and alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4;
R and R′ represent, independently of one another, a hydrogen atom, or an alkyl, cycloalkyl, heterocycloalkyl, aryl, heteroaryl, alkylaryl or alkylheteroaryl group.

3. A compound according to claim 1, wherein R₄ is chosen from the groups of formulae (a), (b) and (c) where X represents a ring member —C(R₆)(R₇)—, in which R₆ is chosen from a halogen atom, or a fused or nonfused cycloalkyl or heterocycloalkyl group located in the spiro position on the ring of formula (a) to which it is attached.

4. A compound according to claim 1, wherein R₄ is chosen from the groups of formulae (a), (b) and (c) where X represents a ring member —C(R₆)(R₇)—, in which R₆ is chosen from —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉ and —C(═NH)—NR₈R₉.

5. A compound to claim 1, wherein R₄ is chosen from the groups of formulae (a), (b) and (c) where X represents a ring member —C(R₆)(R₇)—, in which the alkyl, cycloalkyl, heterocycloalkyl, aryl or heteroaryl groups are optionally substituted with 1 or more groups chosen from R, R′, OCOR, COR, OCONRR′ and NRCOOR′.

6. A compound according to claim 1, wherein R₄ is chosen from the groups of formulae (a), (b) and (c) where X represents a ring member —C(R₆)(R₇)—, in which the cycloalkyl or heterocycloalkyl groups are optionally fused with an aryl or heteroaryl group.

7. A compound according to claim 1, wherein R₄ is chosen from the groups of formulae (a), (b) and (c) where X represents a ring member —C(R₆)(R₇)—, in which R₈ and R₉, chosen independently of one another, represent alkyl and aryl groups which are optionally substituted with one or more groups chosen from the groups R, R′, OCOR, COR, OCONRR′ or NRCOOR′.

8. A compound of claim 1, wherein R₄ is chosen from the groups of formulae (a), (b) and (c) where X represents a ring member —C(R₆)(R₇)—, in which R and R′ can together form a cycloalkyl or a heterocycloalkyl.

9. A compound of claim 1, wherein R₇ is hydrogen.

10. A compound of claim 1, wherein R₄ represents the group of formula a) where p=2 as defined below:

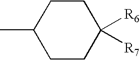

11. A compound of claim 1, wherein R₄ is chosen from the groups of formulae (a), (b) and (c), optionally mono- or polysubstituted with an aryl or heteroaryl group, where X represents a ring member —N(R₁₀)— in which
R₁₀ is chosen from:
a group —CO—NR₈R₉, —COOR₈,
a group —(CH₂)ₓ—OR₈, —(CH₂)ₓ—COOR₈, —(CH₂)—NR₈R₉, —(CH₂)—CO—NR₈R₉ or —(CH₂)—NR₈—COR₉, in which x=1, 2, 3 or 4,
a cycloalkyl or heterocycloalkyl group fused with an aryl or heteroaryl group,
a cycloalkyl, heterocycloalkyl, aryl, heteroaryl, alkylaryl, alkylheteroaryl, —CO-cycloalkyl, —CO-heterocycloalkyl, —CO-heteroaryl, —CO-alkylaryl, —CO-alkylheteroaryl, —CS-alkyl, —CS-cycloalkyl, —CS-heterocycloalkyl, —CS-aryl, —CS-heteroaryl, —CS-alkylaryl, —CS-alkylheteroaryl, —CS—NR₈R₉, —C(═NH)—NR₈R₉, —SO₂-cycloalkyl, —SO₂-heterocycloalkyl, —SO₂-heteroaryl, —SO₂-alkylaryl, —SO₂-alkylheteroaryl or —SO₂—NR₈R₉ group;
or else R₁₀ forms, with the nitrogen atom to which it is attached and a carbon atom located in any position of the cyclic structure of formula (a), but not adjacent to said nitrogen atom, a bridge comprising from 3 to 5 members;
R₈ and R₉ are chosen, independently of one another, from a hydrogen atom, and alkyl, cycloalkyl, heterocycloalkyl, aryl, heteroaryl, alkylaryl, alkylheteroaryl, —CO-alkyl, —CO-cycloalkyl, —CO-heterocycloalkyl, —CO-aryl, —CO-heteroaryl, —CO-alkylaryl, —CO-alkylheteroaryl, —SO₂-alkyl, —SO₂-cycloalkyl, —SO₂-heterocycloalkyl, —SO₂-aryl, —SO₂-heteroaryl, —SO₂-alkylaryl, —SO₂-alkylheteroaryl, —C(═NH)—NRR′, —COOR, —CO—NRR′, —CS—NRR′ and —(CH₂)ₙ—OR groups, where x=0, 1, 2, 3 or 4;
R and R′ represent, independently of one another, a hydrogen atom, or an alkyl, cycloalkyl, heterocycloalkyl, aryl, heteroaryl, alkylaryl or alkylheteroaryl group.

12. A compound of claim 1, wherein
R₄ is chosen from the groups of formulae (a), (b) and (c) optionally substituted with an oxo group where X represents a ring member —N(R₁₀).

13. A compound of claim 1, wherein R₄ is chosen from the groups of formulae (a), (b) and (c) where X represents a ring member —N(R₁₀)—, in which
R₈ and R₉ together form a cycloalkyl or a heterocycloalkyl.

14. A compound of claim 1, wherein R₄ is chosen from the groups of formulae (a), (b) and (c) where X represents a ring member —N(R₁₀)—, in which
R₁₀ is —(CH₂)ₓ—COR₈, in which x=1, 2, 3 or 4.

15. A compound of claim 1, wherein R₄ is chosen from the groups of formulae (a), (b) and (c) where X represents a ring member —N(R₁₀)—, in which the alkyl, cycloalkyl, heterocycloalkyl, aryl or heteroaryl groups are optionally substituted with one or more groups chosen from R, R′ OCOR, COR, OCONRR′ or NRCOOR′.

16. A compound of claim 1, wherein R₄ is chosen from the groups of formulae (a), (b) and (c) where X represents a ring member —N(R₁₀)—, in which the cycloalkyl or heterocycloalkyl groups are optionally fused with an aryl or heteroaryl group.

17. A compound of claim 1, wherein R₄ represents the group of formula a) where p=2 as defined below:

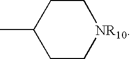

18. A compound according to claim 1, wherein R₁ represents an alkyl, cycloalkyl or heterocycloalkyl group,
in the form of a base or of an addition salt with an acid, and also in the form of a hydrate or of a solvate.

19. A compound of claim 1, wherein R₂ is chosen from the following groups: —CO—R₁₅, —CO—NR₁₆R₁₇, —CO—NR₁₅—NR₁₆R₁₇, —CO-aryl, —CO-heteroaryl, —CO— (CH₂)ₓ—NR₁₆R₁₇, —(CH₂)ₙ—NR₁₆R₁₇, —(CH₂)ₓ—OH, —(CH₂)ₓ-aryl, —(CH₂)ₓ-heteroaryl, —(CH₂)ₓ—CO—R₁₅ and —(CH₂)ₙ—CO—NR₁₆R₁₇, in which:
x=0, 1, 2, 3 or 4 and x′=1, 2, 3 or 4,
R₁₅ represents a hydrogen atom, or an alkyl, cycloalkyl or alkoxy group, and
R₁₆ and R₁₇, which may be identical to or different from one another, represent a hydrogen atom, or an alkyl, cycloalkyl or alkoxy group, or else R₁₆ and R₁₇ form, together with the nitrogen atom to which they are attached, a mono- or bicyclic structure containing from 4 to 10 ring members and optionally comprising 1 to 3 additional hetero atoms and/or 1 to 3 ethylenic or acetylenic unsaturations, this ring being optionally substituted in any positions with 1 to 3 groups chosen from halogen atoms, and hydroxyl, alkyl, cycloalkyl and alkoxy groups,
in the form of a base or of an addition salt with an acid, and also in the form of a hydrate or of a solvate.

20. A compound of claim 1, wherein R₂ represents a group —CO—NR₁₆R₁₇, where R₁₆ and R₁₇ represent alkyl or alkoxy groups,
in the form of a base or of an addition salt with an acid, and also in the form of a hydrate or of a solvate.

21. A compound of claim 1, wherein R₃ represents 1 to 3 groups, which may be identical to or different from one another, chosen from halogen atoms,
in the form of a base or of an addition salt with an acid, and also in the form of a hydrate or of a solvate.

22. A compound of claim 1, wherein R₅ represents a hydrogen atom,
in the form of a base or of an addition salt with an acid, and also in the form of a hydrate or of a solvate.

23. A compound of claim 1, wherein Rₐ=R_{a′} represent hydrogen atoms, or alkyl groups comprising from 1 to 4 carbon atoms,
in the form of a base or of an addition salt with an acid, and also in the form of a hydrate or of a solvate.

24. A compound of claim 1 selected from the group consisting of:
N-{1-[N-(4-aminocyclohexyl)-4-chloro-D-phenylalanyl]piperidin-4-yl}-N-cyclohexyl-N′,N′-diethylurea
N-{1-[N-(cis-4-aminocyclohexyl)-4-chloro-D-phenylalanyl]piperidin-4-yl}-N-cyclohexyl-1,3-dihydro-2H-isoindole-2-carboxamide
N-{1-[N-(cis-4-aminocyclohexyl)-4-chloro-D-phenylalanyl]piperidin-4-yl}-N-cyclohexyl-2,5-dimethylpyrrolidine-1-carboxamide
N-{1-[N-(cis-4-aminocyclohexyl)-4-chloro-D-phenylalanyl]piperidin-4-yl}-N-cyclohexyl-N′,N′-dimethylurea
N-{1-[N-(cis-4-aminocyclohexyl)-4-chloro-D-phenylalanyl]piperidin-4-yl}-N-cyclohexyl-N′-methoxy-N′-methylurea
N-{1-[N-(cis-4-aminocyclohexyl)-4-chloro-D-phenylalanyl]piperidin-4-yl}-N-cyclohexyl-2,5-dimethyl-2,5-dihydro-1H-pyrrole-1-carboxamide
N-{1-[N-(cis-4-aminocyclohexyl)-4-chloro-D-phenylalanyl]piperidin-4-yl}-N-cycloheptyl-N′,N′-diethylurea
N-[1-[N-(cis-4-aminocyclohexyl)-4-chloro-D-phenylalanyl]piperidin-4-yl}-N-cyclooctyl-N′,N′,-diethylurea
N-{1-[N-(cis-4-aminocyclohexyl)-4-chloro-D-phenylalanyl]piperidin-4-yl}-N-cyclohexyl-N′-(2,2,2-trifluoroethyl)urea
N-{1-[N-(cis-4-aminocyclohexyl)-4-chloro-D-phenylalanyl]-3-methylpiperidin-4-yl}-N-cyclohexyl-N′,N′-diethylurea (trans)
N-{1-[N-(trans-4-aminocyclohexyl)-4-chloro-D-phenylalanyl]-3-methylpiperidin-4-yl}]-N-cyclohexyl-N′,N′-diethylurea (trans)
N-{1-[N-(cis-4-aminocyclohexyl)-4-chloro-D-phenylalanyl]piperidin-4-yl}-N-cyclohexyl-N′-ethyl-N′-isopropylurea
N-(cis-4-{[(1R)-1-(4-chlorobenzyl)-2-(4-{cyclohexyl[(diethylamino)carbonyl]amino}piperidin-1-yl)-2-oxoethyl]amino}cyclohexyl)-2,2,2-trifluoroacetamide
N-(trans-4-{[(1R)-1-(4-chlorobenzyl)-2-(4-{cyclohexyl[(diethylamino)carbonyl]amino}piperidin-1-yl)-2-oxoethyl]amino}cyclohexyl)-2,2,2-trifluoroacetamide
N-{1-[N-(1-benzoylpiperidin-4-yl)-4-chloro-D-phenylalanyl]piperidin-4-yl}-N-cyclohexyl-N′,N′-diethylurea
N-{1-[N-(trans-4-aminocyclohexyl)-4-chloro-D-phenylalanyl]piperidin-4-yl}-N-cyclohexyl-N′,N′-bis(2-fluoroethyl)urea
(2R,5S)—N-{1-[N-(cis-4-aminocyclohexyl)-4-chloro-D-phenylalanyl]piperidin-4-yl)-N-cyclohexyl-2,5-dimethylpyrrolidine-1-carboxamide
(2R,5S)-N-(1-{4-chloro-N-[cis-4-(dimethylamino)cyclohexyl]-D-phenylalanyl}piperidin-4-yl)-N-cyclohexyl-2,5-dimethylpyrrolidine-1-carboxamide
4-{[(1R)-1-(4-chlorobenzyl)-2-(4-{cyclohexyl[(dimethylamino)carbonyl]amino}piperidin-1-yl)-2-oxoethyl]amino}-N,N-dimethylpiperidine-1-carboxamide
4-{([(1R)-1-(4-chlorobenzyl)-2-(4-{cyclohexyl[(dimethylamino)carbonyl]amino}piperidin-1-yl)-2-oxoethyl]amino}-N,N-diethylpiperidine-1-carboxamide
N-(1-{4-chloro-N-[1-(pyrrolidin-1-ylcarbonyl)piperidin-4-yl]-D-phenylalanyl]piperidin-4-yl)-N-cyclohexyl-N′,N′-dimethylurea
N-(1-{4-chloro-N-[1-(piperidin-1-ylcarbonyl)piperidin-4-yl]-D-phenylalanyl}piperidin-4-yl)-N-cyclohexyl-N′,N′-dimethylurea
N-(1-{4-chloro-N-[1-(morpholin-4-ylcarbonyl)piperidin-4-yl]-D-phenylalanyl}piperidin-4-yl)-N-cyclohexyl-N′,N′-dimethylurea
4-{[(1R)-1-(4-chlorobenzyl)-2-(4-{cyclohexyl[(dimethylamino)carbonyl]amino}piperidin-1-yl)-2-oxoethyl]amino}-N-phenylpiperidine-1-carboxamide
4-{[(1R)-1-(4-chlorobenzyl)-2-(4-[(cyclohexyl[(dimethylamino)carbonyl]amino}piperidin-1-yl)-2-oxoethyl]amino}-N-methyl-N-phenylpiperidine-1-carboxamide
N-benzyl-4-{([(1R)-1-(4-chlorobenzyl)-2-(4-{cyclohexyl[(dimethylamino)carbonyl]amino}piperidin-1-yl)-2-oxoethyl]amino}-N-methylpiperidine-1-carboxamide
N-(1-{4-chloro-N-[1-(trifluoroacetyl)-piperidin-4-yl]-D-phenylalanyl}piperidin-4-yl)-N-cyclohexyl-N′,N′-dimethylurea
N-{1-[N-(1-acetylpiperidin-4-yl)-4-chloro-D-phenylalanyl]piperidin-4-yl}-N-cyclohexyl-N′,N′-dimethylurea
N-{1-[4-chloro-N-(cis-4-hydroxy-4-phenylcyclohexyl)-D-phenylalanyl]piperidin-4-yl}-N-cyclohexyl-N′,N′-dimethylurea
N-{1-[4-chloro-N-(trans-4-hydroxy-4-phenylcyclohexyl)-D-phenylalanyl]piperidin-4-yl}-N-cyclohexyl-N′,N′-dimethylurea
N-(cis-4-{[(1R)-1-(4-chlorobenzyl)-2-(4-{cyclohexyl[(dimethylamino)carbonyl]amino}piperidin-1-yl)-2-oxoethyl]amino}cyclohexyl)-2,2,2-trifluoroacetamide
N-(trans-4-{[(1R)-1-(4-chlorobenzyl)-2-(4-{cyclohexyl[(dimethylamino)carbonyl]amino}piperidin-1-yl)-2-oxoethyl]amino}cyclohexyl)-2,2,2-trifluoroacetamide
N-[1-(4-chloro-N-{cis-4-[(4-fluorophenyl)-amino]cyclohexyl}-D-phenylalanyl)piperidin-4-yl]-N-cyclohexyl-N′,N′-dimethylurea
N-[1-(4-chloro-N-{trans-4-[(4-fluorophenyl)-amino]cyclohexyl}-D-phenylalanyl)piperidin-4-yl]-N-cyclohexyl-N′,N′-dimethylurea
N-[1-(4-chloro-N-{cis-4-[(2-hydroxyphenyl)amino]cyclohexyl}-D-phenylalanyl)-piperidin-4-yl]-N-cyclohexyl-N′,N′-dimethylurea
N-[1-(4-chloro-N-{trans-4-[(2-hydroxyphenyl)amino]cyclohexyl}-D-phenylalanyl)-piperidin-4-yl]-N-cyclohexyl-N′,N′-dimethylurea
N-{1-[4-chloro-N-(4-methoxycyclohexyl)-D-phenylalanyl]-3-methylpiperidin-4-yl}-N-cyclohexyl-N′,N′-dimethylurea (trans)
N-{1-[4-chloro-N-(4-phenylcyclohexyl)-D-phenylalanyl]-3-methylpiperidin-4-yl}-N-cyclohexyl-N′,N′-dimethylurea (trans)
N-{1-[N-(1-acetylpiperidin-4-yl)-4-chloro-D-phenylalanyl]-3-methylpiperidin-4-yl}-N-cyclohexyl-N′,N′-dimethylurea (trans)
N-(4-{[(1R)-1-(4-chlorobenzyl)-2-(4-(cyclohexyl[(dimethylamino)carbonyl]amino}-3-methylpiperidin-1-yl)-2-oxoethyl]amino}cyclohexyl)-acetamide (trans)
N-(1-{4-chloro-N-[1-(trifluoroacetyl)-piperidin-4-yl]-D-phenylalanyl}-3-methylpiperidin-4-yl)-N-cyclohexyl-N′,N′-dimethylurea (trans)
N-(4-{[(1R)-1-(4-chlorobenzyl)-2-(4-{cyclohexyl[(dimethylamino)carbonyl]amino}-3-methylpiperidin-1-yl)-2-oxoethyl]amino}cyclohexyl)-2,2,2-trifluoroacetamide (trans)
N-{1-[1N-(1-benzoylpiperidin-4-yl)-4-chloro-D-phenylalanyl]-3-methylpiperidin-4-yl}-N-cyclohexyl-N′,N′-dimethylurea
N-(1-(4-chloro-N-[1-(methylsulphonyl)-piperidin-4-yl]-D-phenylalanyl}-3-methylpiperidin-4-yl)-N-cyclohexyl-N′,N′-dimethylurea (trans)
N-1-[4-chloro-N-(4-hydroxy-4-phenylcyclohexyl)-D-phenylalanyl]-3-methylpiperidin-4-yl}-N-cyclohexyl-N′,N′-dimethylurea (trans)
N-[1-(4-chloro-N-{4-[(4-fluorophenyl)-amino]cyclohexyl}-D-phenylalanyl)-3-methylpiperidin-4-yl]-N-cyclohexyl-N′,N′-dimethylurea
N-{1-[1N-(cis-4-aminocyclohexyl)-4-chloro-D-phenylalanyl]-3-methylpiperidin-4-yl]-N-cyclohexyl-N′,N′-dimethylurea (trans)
N-[1-(4-chloro-N-{cis-4-[(2-methoxyphenyl)-amino]cyclohexyl}-D-phenylalanyl)piperidin-4-yl]-N-cyclohexyl-N′,N′-dimethylurea
N-[1-(4-chloro-N-{trans-4-[(2-methoxyphenyl)amino]cyclohexyl}-D-phenylalanyl)-piperidin-4-yl]-N-cyclohexyl-N′,N′-dimethylurea
N-(cis-4-{[1R)-1-(4-chlorobenzyl)-2-(4-{cyclohexyl[(dimethylamino)carbonyl]amino}piperidin-1-yl)-2-oxoethyl]amino}cyclohexyl)acetamide
N-(trans-4-{[(R)-1-(4-chlorobenzyl)-2-(4-{cyclohexyl[(dimethylamino)carbonyl]amino}piperidin-1-yl)-2-oxoethyl]amino}cyclohexyl)acetamide
N-(1-{4-chloro-N-[cis-4-(4-hydroxy-phenyl)cyclohexyl]-D-phenylalanyl}-3-methylpiperidin-4-yl)-N-cyclohexyl-N′,N′-dimethylurea
N-(1-{4-chloro-N-[trans-4-(4-hydroxyphenyl)-cyclohexyl]-D-phenylalanyl}-3-methylpiperidin-4-yl)-N-cyclohexyl-N′,N′-dimethylurea
N-(1-{4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea (trans)
N-{[1-[4-chloro-N-(1-isonicotinoylpiperidin-4-yl)-D-phenylalanyl]-3-methylpiperidin-4-yl}-N-cyclohexyl-N′,N′-dimethylurea (trans)
N-(1-{4-chloro-N-[cis-4-(1,3-dihydro-2H-isoindol-2-yl)cyclohexyl]-D-phenylalanyl}-3-methyl-piperidin-4-yl)-N-cyclohexyl-N′,N′-dimethylurea (trans)
N-{1-[4-chloro-N-(2-phenylpiperidin-4-yl)-D-phenylalanyl]-3-methylpiperidin-4-yl}-N-cyclohexyl-N′,N′-dimethylurea (trans) and
N-(1-{4-chloro-N-[4-(3-oxopiperazin-1-yl)cyclohexyl]-D-phenylalanyl}-3-methylpiperidin-4-yl)-N-cyclohexyl-N′,N′-dimethylurea (trans).

25. A compound of claim 1 selected from the group consisting of:
N-{1-[N-(4-aminocyclohexyl)-4-chloro-D-phenylalanyl]piperidin-4-yl}-N-cyclohexyl-N′,N′,-diethylurea
N-{1-[N-(cis-4-aminocyclohexyl)-4-chloro-D-phenylalanyl]piperidin-4-yl}-N-cyclohexyl-1,3-dihydro-2H-isoindole-2-carboxamide
N-[1-[N-(cis-4-aminocyclohexyl)-4-chloro-D-phenylalanyl]piperidin-4-yl}-N-cyclohexyl-2,5-dimethylpyrrolidine-1-carboxamide
N-{1-[N-(cis-4-aminocyclohexyl)-4-chloro-D-phenylalanyl]piperidin-4-yl}-N-cyclohexyl-N′,N′-dimethylurea
N-{1-[N-(cis-4-aminocyclohexyl)-4-chloro-D-phenylalanyl]piperidin-4-yl}-N-cyclohexyl-N′-methoxy-N′-methylurea
N-[1-[N-(cis-4-aminocyclohexyl)-4-chloro-D-phenylalanyl]piperidin-4-yl}-N-cyclohexyl-2,5-dimethyl-2,5-dihydro-1H-pyrrole-1-carboxamide
N-[1-[N-(cis-4-aminocyclohexyl)-4-chloro-D-phenylalanyl]piperidin-4-yl}-N-cyclobutyl-N′,N′-diethylurea
N-{1-[N-(cis-4-aminocyclohexyl)-4-chloro-D-phenylalanyl]piperidin-4-yl}-N-cyclopentyl-N′,N′-diethylurea
N-{1-[N-(cis-4-aminocyclohexyl)-4-chloro-D-phenylalanyl]piperidin-4-yl}-N-cycloheptyl-N′,N′-diethylurea
N-{1-[N-(cis-4-aminocyclohexyl)-4-chloro-D-phenylalanyl]piperidin-4-yl}-N-cyclooctyl-N′,N′-diethylurea
N-{1-[N-(cis-4-aminocyclohexyl)-4-chloro-D-phenylalanyl]piperidin-4-yl}-N′,N′-diethyl-N-phenylurea
N-{1-[N-(cis-4-aminocyclohexyl)-4-chloro-D-phenylalanyl]piperidin-4-yl}-N-cyclohexyl-N′-(2,2,2-trifluoroethyl)urea
N-{1-[4-chloro-N-(4-hydroxycyclohexyl)-D-phenylalanyl]piperidin-4-yl}-N-cyclohexyl-N′,N′-diethylurea
N-{1-[N-(cis-4-aminocyclohexyl)-4-chloro-D-phenylalanyl]-3-methylpiperidin-4-yl}-N-cyclohexyl-N′,N′-diethylurea (trans)
N-{1-[N-(cis-4-aminocyclohexyl)-4-chloro-D-phenylalanyl]-3-methylpiperidin-4-yl}-N-cyclohexyl-N′,N′-diethylurea (cis)
N-{1-[N-(trans-4-aminocyclohexyl)-4-chloro-D-phenylalanyl]-3-methylpiperidin-4-yl}-N-cyclohexyl-N′,N′-diethylurea (trans)
N-{1-[N-(trans-4-aminocyclohexyl)-4-chloro-D-phenylalanyl]-3-methylpiperidin-4-yl}-N-cyclohexyl-N′,N′-diethylurea (cis)
N-{1-[N-(cis-4-aminocyclohexyl)-4-chloro-D-phenylalanyl]piperidin-4-yl}-N′,N′-diethyl-N-(tetrahydro-2H-pyran-4-yl)urea
N-{1-[N-(cis-4-aminocyclohexyl)-4-chloro-D-phenylalanyl]piperidin-4-yl}-N′,N′-diethyl-N-piperidin-4-ylurea
N-{1-[N-(cis-4-aminocyclohexyl)-4-chloro-D-phenylalanyl]piperidin-4-yl}-N-cyclohexyl-N′-ethyl-N′-isopropylurea
N-[1-[N-(cis-4-aminocyclohexyl)-4-chloro-D-phenylalanyl]piperidin-4-yl}-N-cyclohexyl-2,2-dimethylhydrazinecarboxamide
N-(cis-4-{[(1R)-1-(4-chlorobenzyl)-2-(4-{cyclohexyl[(diethylamino)carbonyl]amino]piperidin-1-yl)-2-oxoethyl]amino}cyclohexyl)-2,2,2-trifluoroacetamide
N-(trans-4-{[(1R)-1-(4-chlorobenzyl)-2-(4-{cyclohexyl[(diethylamino)carbonyl]amino}piperidin-1-yl)-2-oxoethyl]amino}cyclohexyl)-2,2,2-trifluoroacetamide and
N-[1-[N-(trans-4-aminocyclohexyl)-4-chloro-D-phenylalanyl]piperidin-4-yl}-N-cyclohexyl-N′,N′-bis (2-fluoroethyl)urea.

26. A compound of claim 1 selected from the group consisting of:
N-[(1R)-1-(4-chlorobenzyl)-2-(4-{cyclohexyl-[2-(diethylamino)ethyl]amino}piperidin-1-yl)-2-oxoethyl]cyclohexane-1,4-diamine
N-(1-{4-chloro-N-[cis-4-(dimethylamino)-cyclohexyl]-D-phenylalanyl}piperidin-4-yl)-N-cyclohexyl-3,4-difluorobenzamide
N-(1-{4-chloro-N-[cis-4-(dimethylamino)-cyclohexyl]-D-phenylalanyl}piperidin-4-yl)-N-cycloheptyl-N′,N′-dimethylurea
(2R,5S)—N-{1-[N-(cis-4-aminocyclohexyl)-4-chloro-D-phenylalanyl]piperidin-4-yl]-N-cyclohexyl-2,5-dimethylpyrrolidine-1-carboxamide
(2R,5S)—N-(1-[4-chloro-N-[cis-4-(dimethylamino)cyclohexyl]-D-phenylalanyl}piperidin-4-yl)-N-cyclohexyl-2,5-dimethylpyrrolidine-1-carboxamide
N-{1-[4-chloro-N-(cis-4-hydroxy-4-phenylcyclohexyl)-D-phenylalanyl]piperidin-4-yl}-N-cyclohexyl-N′,N′-dimethylurea
N-{1-[4-chloro-N-(trans-4-hydroxy-4-phenylcyclohexyl)-D-phenylalanyl]piperidin-4-yl}-N-cyclohexyl-N′,N′-dimethylurea
N-(cis-4-{[(1R)-1-(4-chlorobenzyl)-2-(4-{cyclohexyl[(dimethylamino)carbonyl]amino}piperidin-1-yl)-2-oxoethyl]amino}cyclohexyl)-2,2,2-trifluoro-acetamide
N-(trans-4-{[(1R)-1-(4-chlorobenzyl)-2-(4-{cyclohexyl[(dimethylamino)carbonyl]amino}piperidin-1-yl)-2-oxoethyl]amino}cyclohexyl)-2,2,2-trifluoro-acetamide
N-[1-(4-chloro-N-{cis-4-[(4-fluorophenyl)-amino]cyclohexyl}-D-phenylalanyl)piperidin-4-yl]-N-cyclohexyl-N′,N′-dimethylurea
N-[1-(4-chloro-N-{trans-4-[(4-fluorophenyl)-amino]cyclohexyl}-D-phenylalanyl)piperidin-4-yl]-N-cyclohexyl-N′,N′-dimethylurea
N-{1-[4-chloro-N-(4-methoxycyclohexyl)-D-phenylalanyl]piperidin-4-yl}-N-cyclohexyl-N′,N′-dimethylurea
N-[1-(4-chloro-N-{cis-4-[(4-methoxyphenyl)-amino]cyclohexyl}-D-phenylalanyl)piperidin-4-yl]-N-cyclohexyl-N′,N′-dimethylurea
N-[1-(4-chloro-N-{trans-4-[(4-methoxyphenyl)-amino]cyclohexyl}-D-phenylalanyl)piperidin-4-yl]-N-cyclohexyl-N′,N′-dimethylurea
N-[1-(4-chloro-N-{cis-4-[(2-hydroxyphenyl)-amino]cyclohexyl}-D-phenylalanyl)piperidin-4-yl]-N-cyclohexyl-N′,N′-dimethylurea
N-[1-(4-chloro-N-{trans-4-[(2-hydroxyphenyl)-amino]cyclohexyl}-D-phenylalanyl)piperidin-4-yl]-N-cyclohexyl-N′,N′-dimethylurea
N-[1-(4-chloro-N-(4-[(dimethylamino)methyl]-4-phenylcyclohexyl}-D-phenylalanyl)piperidin-4-yl]-N-cyclohexyl-N′,N′-dimethylurea
(2S,5S)—N-(1-{4-chloro-N-[cis-4-(dimethylamino)cyclohexyl]-D-phenylalanyl}piperidin-4-yl)-N-cyclohexyl-2,5-dimethylpyrrolidine-1-carboxamide
(2R,5R)—N-(1-{4-chloro-N-[cis-4-(dimethylamino)cyclohexyl]-D-phenylalanyl]piperidin-4-yl)-N-cyclohexyl-2,5-dimethylpyrrolidine-1-carboxamide
N-{1-[4-chloro-N-(4-methoxycyclohexyl)-D-phenylalanyl]-3-methylpiperidin-4-yl]-N-cyclohexyl-N′,N′-dimethylurea
N-(1-[4-chloro-N-(4-phenylcyclohexyl)-D-phenylalanyl]-3-methylpiperidin-4-yl}-N-cyclohexyl-N′,N′-dimethylurea
N-(4-{[(1R)-1-(4-chlorobenzyl)-2-(4-{cyclohexyl[(dimethylamino)carbonyl]amino}-3-methylpiperidin-1-yl)-2-oxoethyl]amino}cyclohexyl)-acetamide
N-(4-{[(1R)-1-(4-chlorobenzyl)-2-(4-[cyclohexyl[(dimethylamino)carbonyl]amino}-3-methylpiperidin-1-yl)-2-oxoethyl]amino}cyclohexyl)-2,2,2-trifluoroacetamide
N-{1-[4-chloro-N-(4-hydroxy-4-phenylcyclohexyl)-D-phenylalanyl]-3-methylpiperidin-4-yl]-N-cyclohexyl-N′,N′-dimethylurea
N-[1-(4-chloro-N-{4-[(4-fluorophenyl)amino]-cyclohexyl}-D-phenylalanyl)-3-methylpiperidin-4-yl]-N-cyclohexyl-N′,N′-dimethylurea
N-(1-{4-chloro-N-[cis-4-(dimethylamino)-cyclohexyl]-D-phenylalanyl}piperidin-4-yl)-N-cyclohexyl-N′,N′-dimethylurea
N-{1-[N-(cis-4-aminocyclohexyl)-4-chloro-D-phenylalanyl]-3-methylpiperidin-4-yl}]-N-cyclohexyl-N′,N′-dimethylurea
N-[1-(4-chloro-N-[cis-4-[(2-methoxyphenyl)-amino]cyclohexyl}-D-phenylalanyl)piperidin-4-yl]-N-cyclohexyl-N′,N′-dimethylurea
N-[1-(4-chloro-N-{cis-4-[(2-methoxyphenyl)-amino]cyclohexyl}-D-phenylalanyl)piperidin-4-yl]-N-cyclohexyl-N′,N′-dimethylurea
N-(cis-4-{[(1R)-1-(4-chlorobenzyl)-2-(4-[cyclohexyl[(dimethylamino)carbonyl]amino}piperidin-1-yl)-2-oxoethyl]amino}cyclohexyl)acetamide
N-(trans-4-{([(1R)-1-(4-chlorobenzyl)-2-(4-{cyclohexyl[(dimethylamino)carbonyl]amino}piperidin-1-yl)-2-oxoethyl]amino}cyclohexyl)acetamide
N-(1-{4-chloro-N-[cis-4-(4-hydroxyphenyl)-cyclohexyl]-D-phenylalanyl}-3-methylpiperidin-4-yl)-N-cyclohexyl-N′,N′-dimethylurea
N-(1-{4-chloro-N-[trans-4-(4-hydroxyphenyl)-cyclohexyl]-D-phenylalanyl}-3-methylpiperidin-4-yl)-N-cyclohexyl-N′,N′-dimethylurea
N-(1-{4-chloro-N-[4-(2-oxo-1,3-oxazolidin-3-yl)cyclohexyl]-D-phenylalanyl}-3-methylpiperidin-4-yl)-N-cyclohexyl-N′,N′-dimethylurea
N-(1-(4-chloro-N-[cis-4-(1,3-dihydro-2H-isoindol-2-yl)cyclohexyl]-D-phenylalanyl}-3-methylpiperidin-4-yl)-N-cyclohexyl-N′,N′-dimethylurea and
N-(1-{4-chloro-N-[4-(3-oxopiperazin-1-yl)cyclohexyl]-D-phenylalanyl}-3-methylpiperidin-4-yl)-N-cyclohexyl-N′,N′-dimethylurea.

27. A compound of claim 1 selected from the group consisting of:
N-[1-(N-8-azabicyclo[3.2.1]oct-3-yl-4-chloro-D-phenylalanyl)piperidin-4-yl]-N-cyclohexyl-N′,N′-diethylurea
N-[1-(N-1-azabicyclo[2.2.2]oct-3-yl-4-chloro-D-phenylalanyl)piperidin-4-yl]-N-cyclohexyl-N′,N′-diethylurea
N-[1-(N-8-azabicyclo[3.2.1]oct-3-yl-4-chloro-D-phenylalanyl)piperidin-4-yl]-N-cyclobutyl-N′,N′-diethylurea and
N-{1-[N-(1-benzoylpiperidin-4-yl)-4-chloro-D-phenylalanyl]piperidin-4-yl}-N-cyclohexyl-N′,N′-diethylurea.

28. A compound of claim 1 selected from the group consisting of:
4-{[(1R)-1-(4-chlorobenzyl)-2-(4-{cyclohexyl-[(dimethylamino)carbonyl]amino}piperidin-1-yl)-2-oxoethyl]amino}-N,N-dimethylpiperidine-1-carboxamide
4-{[(1R)-1-(4-chlorobenzyl)-2-(4-[cyclohexyl-[(dimethylamino)carbonyl]amino piperidin-1-yl)-2-oxoethyl]amino}-N,N-diethylpiperidine-1-carboxamide
N-(1-{4-chloro-N-[1-(pyrrolidin-1-ylcarbonyl)piperidin-4-yl]-D-phenylalanyl}piperidin-4-yl)-N-cyclohexyl-N′,N′-dimethylurea
N-(1-{4-chloro-N-[1-(piperidin-1-ylcarbonyl)-piperidin-4-yl]-D-phenylalanyl}piperidin-4-yl)-N-cyclohexyl-N′,N′-dimethylurea
N-(1-{4-chloro-N-[1-(morpholin-4-ylcarbonyl)-piperidin-4-yl]-D-phenylalanyl}piperidin-4-yl)-N-cyclohexyl-N′,N′-dimethylurea
4-{[(1R)-1-(4-chlorobenzyl)-2-(4-{cyclohexyl-[(dimethylamino)carbonyl]amino}piperidin-1-yl)-2-oxoethyl]amino}-N-phenylpiperidine-1-carboxamide
4-{[(1R)-1-(4-chlorobenzyl)-2-(4-{cyclohexyl-[(dimethylamino)carbonyl]amino}piperidin-1-yl)-2-oxoethyl]amino}-N-methyl-N-phenylpiperidine-1-carboxamide
N-benzyl-4-{[(1R)-1-(4-chlorobenzyl)-2-(4-{cyclohexyl[(dimethylamino)carbonyl]amino}piperidin-1-yl)-2-oxoethyl]amino}-N-methylpiperidine-1-carboxamide
N-(1-{4-chloro-N-[1-(trifluoroacetyl)-piperidin-4-yl]-D-phenylalanyl}piperidin-4-yl)-N-cyclohexyl-N′,N′-dimethylurea
N-{1-[N-(1-acetylpiperidin-4-yl)-4-chloro-D-phenylalanyl]piperidin-4-yl}-N-cyclohexyl-N′,N′-dimethylurea
N-{1-[N-(1-acetylpiperidin-4-yl)-4-chloro-D-phenylalanyl]-3-methylpiperidin-4-yl)-N-cyclohexyl-N′,N′-dimethylurea
N-(1-{4-chloro-N-[1-(trifluoroacetyl)-piperidin-4-yl]-D-phenylalanyl}-3-methylpiperidin-4-yl)-N-cyclohexyl-N′,N′-dimethylurea
N-{1-[N-[(1-benzoylpiperidin-4-yl}-4-chloro-D-phenylalanyl]-3-methylpiperidin-4-yl}-N-cyclohexyl-N′,N′-dimethylurea
N-(1-{4-chloro-N-[1-(methylsulphonyl)-piperidin-4-yl]-D-phenylalanyl}-3-methylpiperidin-4-yl)-N-cyclohexyl-N′,N′-dimethylurea
N-(1-{4-chloro-N-[1-(methylsulphonyl)-piperidin-4-yl]-D-phenylalanyl}piperidin-4-yl)-N-cyclohexyl-N′,N′-dimethylurea
N-{1-[4-chloro-N-(1-isonicotinoylpiperidin-4-yl)-D-phenylalanyl]-3-methylpiperidin-4-yl}-N-cyclohexyl-N′,N′-dimethylurea and
N-{1-[4-chloro-N-(2-phenylpiperidin-4-yl)-D-phenylalanyl]-3-methylpiperidin-4-yl}-N-cyclohexyl-N′,N′-dimethylurea.

29. A medicament which comprises a compound of claim 1, or an addition salt of this compound with a pharmaceutically acceptable acid, or else a hydrate or a solvate of the compound of formula (I).

30. A pharmaceutical composition which comprises a compound of claim 1, or a pharmaceutically acceptable salt, a hydrate or a solvate of this compound, and also at least one pharmaceutically acceptable excipient.

31. A method of treatment or prevention of a condition selected from obesity, diabetes and sexual dysfunctions that can affect both sexes, in the treatment of cardiovascular diseases, and also in anti-inflammatory uses or in the treatment of alcohol dependency comprising administering to a patient in need thereof an effective amount of a compound of claim 1.

32. The method of claim 31, wherein said sexual dysfunctions consist of erectile dysfunctions.

33. A method for preparing a compound of claim 1, wherein a reductive amination of a compound of formula (V): 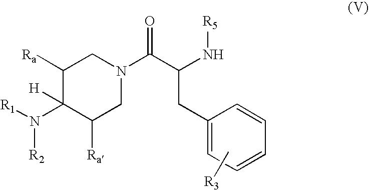
is carried out in the presence of a derivative of the group R₄ of ketone type, R₁, R₂, R₃, R₄, R₅, Rₐ and R_{a′}, being as defined in claim 1.

34. A compound of formulae (VI), (XVIII) or (XIX), in which R₁, R₂, R₃, R₄, R₅, Rₐ and R_{a′} are as defined in claim 1 and Pg represents a protective group: [chemical structure]
